SEQUENCE LISTING (115 sequences)

SEQ ID NO: 1 - protein, 757 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 2 - protein, 4 aa, synthetic construct
GGGS

SEQ ID NO: 3 - protein, 5 aa, synthetic construct
GGGGS

SEQ ID NO: 4 - protein, 32 aa, synthetic construct
SGGSSGGSSGSETPGTSESATPESSGGSSGGS

SEQ ID NO: 5 - protein, 65 aa, synthetic construct
SGGSSGGSSGSETPGTSESATPESSGGSSGGSSSGGSSGGSSGSETPGTSESATPESSGGSSGGS

SEQ ID NO: 6 - protein, 97 aa, synthetic construct
SGGSSGGSSGSETPGTSESATPESSGGSSGGSSGGSSGGSSGSETPGTSESATPESSGGSSGGSSSGGSSGGSSGSETPGTSESATPESSGGSSGGS

SEQ ID NO: 7 - protein, 4 aa, synthetic construct
GGGS

SEQ ID NO: 8 - protein, 8 aa, synthetic construct
GGGSGGGS

SEQ ID NO: 9 - skipped

SEQ ID NO: 10 - protein, 5 aa, synthetic construct
PAPAP

SEQ ID NO: 11 - protein, 7 aa, synthetic construct
PAPAPAP

SEQ ID NO: 12 - protein, 16 aa, synthetic construct
SGSETPGTSESATPES

SEQ ID NO: 13 - protein, 7 aa, unidentified
  source 1..7: note = SV40
PKKKRRV

SEQ ID NO: 14 - protein, 16 aa, unidentified
  source 1..16: note = SV40
KRPAATKKAGQAKKKK

SEQ ID NO: 15 - protein, 795 aa, synthetic construct
MKRTADGSEFESPKKKRKVPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 16 - protein, 795 aa, synthetic construct
MKRTADGSEFESPKKKRKVPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFRYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRKGEAFQCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 17 - protein, 795 aa, synthetic construct
MKRTADGSEFESPKKKRKVPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQRRNEKARARLESINASRAKEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFRYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRKGEAFQCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 18 - protein, 795 aa, synthetic construct
MKRTADGSEFESPKKKRKVPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQRRNEKARARLESINASRAKEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFRYKLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRKGEAFQCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 19 - protein, 795 aa, synthetic construct
MKRTADGSEFESPKKKRKVPKPAVESEFSKVLKKHFPGERFRSSYMKRGGKILRAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQRRNEKARARLESINASRAKEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFRYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGRGKRLPGWDNFFTAKKENRWFIQGLHKAFSDLRTHRSFYVFEVRPERTSITCPKCGHCEVGNRKGEAFQCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 20 - protein, 795 aa, synthetic construct
MKRTADGSEFESPKKKRKVPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFRHTLGRYDGVLKKVQRRNEKARARLESINASRAKEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFRYKLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRKGEAFQCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 21 - protein, 795 aa, synthetic construct
MKRTADGSEFESPKKKRKVPKPAVESEFSKVLKKHFPGERFRSSYMKRGGKILRAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQRRNEKARARLESINASRAKEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFRYKLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGRGKRLPGWDNFFTAKKENRWFIQGLHKAFSDLRTHRSFYVFEVRPERTSITCPKCGHCEVGNRKGEAFQCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 22 - protein, 795 aa, synthetic construct
MKRTADGSEFESPKKKRKVPKPAVESEFSKVLKKHFPGERFRSSYMKRGGKILRAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQRRNEKARARLESINASRAKEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFRYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGRGKRLPGWDNFFTAKKENRWFIQGLHKAFSDLRTHRSFYVFEVRPERTSITCPKCGHCEVGNRKGEAFRCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 23 - protein, 795 aa, synthetic construct
MKRTADGSEFESPKKKRKVPKPAVESEFSKVLKKHFPGERFRSSYMKRGGKILRAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQRRNEKARARLESINASRAKEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSRKKNKRMRRYWRSEKEKAQDALLVTVRIGTDWVVIDVRGLLRNARWRTIAPKDISLNALLDLFTGDPVIDVRRNIVTFRYKLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGRGKRLPGWDNFFTAKKENRWFIQGLHKAFSDLRTHRSFYVFEVRPERTSITCPKCGHCEVGNRKGEAFQCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 24 - protein, 795 aa, synthetic construct
MKRTADGSEFESPKKKRKVPKPAVESEFSKVLKKHFPGERFRSSYMKRGGKILRAQGEEAVVAYLQGKSEEEPPNFQPPAKCHVVTKSRDFAEWPIMKASEAIQRYIYALSTTERAACKPGKSRESHAAWFAATGVSNHGYSHVQGLNLIFRHTLGRYDGVLKKVQRRNEKARARLESINASRAKEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSRKKNKRMRRYWRSEKEKAQDALLVTVRIGTDWVVIDVRGLLRNARWRTIAPKDISLNALLDLFTGDPVIDVRRNIVTFRYKLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGRGKRLPGWDNFFTAKKENRWFIQGLHKAFSDLRTHRSFYVFEVRPERTSITCPKCGHCEVGNRKGEAFQCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 25 - protein, 795 aa, synthetic construct
MKRTADGSEFESPKKKRKVPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSRESHAAWFAATGVSNHGYSHVQGLNLIFRHTLGRYDGVLKKVQRRNEKARARLESINASRAKEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSRKKNKRMRRYWRSEKEKAQDALLVTVRIGTDWVVIDVRGLLRNARWRTIAPKDISLNALLDLFTGDPVIDVRRNIVTFRYKLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFRPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRKGEAFRCLSCGKRCNADLDVATHNLTQVALTGKTMPKREEPRDAQGTAPARKTKKASKSKAPPAEREDQTPAQEPSQTSGGSKRTADGSEFEPKKKRKV

SEQ ID NO: 26 - protein, 795 aa, synthetic construct
MKRTADGSEFESPKKKRKVPKPAVESEFSKVLKKHFPGERFRSSYMKRGGKILRAQGEEAVVAYLQGKSEEEPPNFQPPAKCHVVTKSRDFAEWPIMKASEAIQRYIYALSTTERAACKPGKSRESHAAWFAATGVSNHGYSHVQGLNLIFRHTLGRYDGVLKKVQRRNEKARARLESINASRAKEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSRKKNKRMRRYWRSEKEKAQDALLVTVRIGTDWVVIDVRGLLRNARWRTIAPKDISLNALLDLFTGDPVIDVRRNIVTFRYKLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFRPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGRGKRLPGWDNFFTAKKENRWFIQGLHKAFSDLRTHRSFYVFEVRPERTSITCPKCGHCEVGNRKGEAFQCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 27 - protein, 795 aa, synthetic construct
MKRTADGSEFESPKKKRKVPKPAVESEFSKVLKKHFPGERFRSSYMKRGGKILRAQGEEAVVAYLQGKSEEEPPNFQPPAKCHVVTKSRDFAEWPIMKASEAIQRYIYALSTTERAACKPGKSRESHAAWFAATGVSNHGYSHVQGLNLIFRHTLGRYDGVLKKVQRRNEKARARLESINASRAKEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSRKKNKRMRRYWRSEKEKAQDALLVTVRIGTDWVVIDVRGLLRNARWRTIAPKDISLNALLDLFTGDPVIDVRRNIVTFRYKLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGRGKRLPGWDNFFTAKKENRWFIQGLHKAFSDLRTHRSFYVFEVRPERTSITCPKCGHCEVGNRKGEAFRCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 28 - protein, 795 aa, synthetic construct
MKRTADGSEFESPKKKRKVPKPAVESEFSKVLKKHFPGERFRSSYMKRGGKILRAQGEEAVVAYLQGKSEEEPPNFQPPAKCHVVTKSRDFAEWPIMKASEAIQRYIYALSTTERAACKPGKSRESHAAWFAATGVSNHGYSHVQGLNLIFRHTLGRYDGVLKKVQRRNEKARARLAAINAARAKAGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSRKKNKRMRRYWRSEKEKAQDALLVTVRIGTDWVVIDVRGLLRNARWRTIAPKDISLNALLDLFTGDPVIDVRRNIVTFRYKLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGRGKRLPGWDNFFTAKKENRWFIQGLHKAFSDLRTHRSFYVFEVRPERTSITCPKCGHCEVGNRKGEAFRCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 29 - protein, 795 aa, synthetic construct
MKRTADGSEFESPKKKRKVPKPAVESEFSKVLKKHFPGERFRSSYMKRGGKILRAQGEEAVVAYLQGKSEEEPPNFQPPAKCHVVTKSRDFAEWPIMKASEAIQRYIYALSTTERAACKPGKSRESHAAWFAATGVSNHGYSHVQGLNLIFRHTLGRYDGVLKKVQRRNEKARARLAAINAARAKAGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSRKKNKRMRRYWRSEKEKAQDALLVTVRIGTDWVVIDVRGLLRNARWRTIAPKDISLNALLDLFTGDPVIDVRRNIVTFRYKLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFRPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGRGKRLPGWDNFFTAKKENRWFIQGLHKAFSDLRTHRSFYVFEVRPERTSITCPKCGHCEVGNRKGEAFRCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 30 - protein, 993 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSPKPAVESEFSKVLKKHFPGERFRSSYMKRGGKILRAQGEEAVVAYLQGKSEEEPPNFQPPAKCHVVTKSRDFAEWPIMKASEAIQRYIYALSTTERAACKPGKSRESHAAWFAATGVSNHGYSHVQGLNLIFRHTLGRYDGVLKKVQRRNEKARARLAAINAARAKAGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSRKKNKRMRRYWRSEKEKAQDALLVTVRIGTDWVVIDVRGLLRNARWRTIAPKDISLNALLDLFTGDPVIDVRRNIVTFRYKLDACGTYARKWTLKGKQTKATLDKLTATQTVALVAIALGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFRPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGRGKRLPGWDNFFTAKKENRWFIQGLHKAFSDLRTHRSFYVFEVRPERTSITCPKCGHCEVGNRKGEAFRCLSCGKTCNADLDVATHNLTQVALTGKTMPKREEPRDAQGTAPARKTKKASKSKAPPAEREDQTPAQEPSQTSGGSKRTADGSEFEPKKKRKV

SEQ ID NO: 31 - protein, 1362 aa, synthetic construct
MKRTADGSEFESPKKKRKVPKPAVESEFSKVLKKHFPGERFRSSYMKRGGKILRAQGEEAVVAYLQGKSEEEPPNFQPPAKCHVVTKSRDFAEWPIMKASEAIQRYIYALSTTERAACKPGKSRESHAAWFAATGVSNHGYSHVQGLNLIFRHTLGRYDGVLKKVQRRNEKARARLAAINAARAKAGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSRKKNKRMRRYWRSEKEKAQDALLVTVRIGTDWVVIDVRGLLRNARWRTIAPKDISLNALLDLFTGDPVIDVRRNIVTFRYKLDACGTYARKWTLKGKQTKATLDKLTATQTVALVAIALGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFRPAPKKGAKKKAPVEVMRKDRTWARAYKPRLSVEAQKLKNEALWALKRTSPEYKKLSRRKEELCRRSINYVIEKTRRRTQCQIVIPVIEDLNVRFFHGRGKRLPGWDNFFTAKKENRWFIQGLHKAFSDLRTHRSFYVFEVRPERTSITCPKCGHCEVGNRKGEAFRCLSCGKTCNADLDVATHNLTQVALTGKTMPKREEPRDAQGTAPARKTKKASKSKAPPAEREDQTPAQEPSQTSGSPKKKRKVKRPAATKKAGQAKKKKGSYPYDVPDYAYPYDVPDYAYPYDVPDYAGSEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 32 - RNA, 20 nt, synthetic construct
  source 1..20: mol_type = other RNA
ctgaagctgacagcattcgg

SEQ ID NO: 33 - RNA, 20 nt, synthetic construct
  source 1..20: mol_type = other RNA
cagcattcgggccgagatgt

SEQ ID NO: 34 - RNA, 20 nt, synthetic construct
  source 1..20: mol_type = other RNA
gggccgagatgtctcgctcc

SEQ ID NO: 35 - RNA, 20 nt, synthetic construct
  source 1..20: mol_type = other RNA
ctcgctccgtggccttagct

SEQ ID NO: 36 - RNA, 20 nt, synthetic construct
  source 1..20: mol_type = other RNA
cgctccgtggccttagctgt

SEQ ID NO: 37 - RNA, 20 nt, synthetic construct
  source 1..20: mol_type = other RNA
gtggccttagctgtgctcgc

SEQ ID NO: 38 - RNA, 20 nt, synthetic construct
  source 1..20: mol_type = other RNA
ccttagctgtgctcgcgcta

SEQ ID NO: 39 - RNA, 20 nt, synthetic construct
  source 1..20: mol_type = other RNA
gctgtgctcgcgctactctc

SEQ ID NO: 40 - RNA, 20 nt, synthetic construct
  source 1..20: mol_type = other RNA
cgctactctctctttctggc

SEQ ID NO: 41 - RNA, 20 nt, synthetic construct
  source 1..20: mol_type = other RNA
ctggcctggaggctatccag

SEQ ID NO: 42 - RNA, 20 nt, synthetic construct
  source 1..20: mol_type = other RNA
tggcctggaggctatccagc

SEQ ID NO: 43 - RNA, 20 nt, synthetic construct
  source 1..20: mol_type = other RNA
cctggaggctatccagcgtg

SEQ ID NO: 44 - RNA, 20 nt, synthetic construct
  source 1..20: mol_type = other RNA
aagctagtctagtgcaagct

SEQ ID NO: 45 - RNA, 20 nt, synthetic construct
  source 1..20: mol_type = other RNA
aagctaacagttgcttttat

SEQ ID NO: 46 - RNA, 20 nt, synthetic construct
  source 1..20: mol_type = other RNA
cttttatcacaggctccagg

SEQ ID NO: 47 - RNA, 20 nt, synthetic construct
  source 1..20: mol_type = other RNA
tatcacaggctccaggaagg

SEQ ID NO: 48 - RNA, 20 nt, synthetic construct
  source 1..20: mol_type = other RNA
atcacaggctccaggaaggg

SEQ ID NO: 49 - RNA, 20 nt, synthetic construct
  source 1..20: mol_type = other RNA
tcacaggctccaggaagggt

SEQ ID NO: 50 - RNA, 20 nt, synthetic construct
  source 1..20: mol_type = other RNA
aggaagggtttggcctctga

SEQ ID NO: 51 - RNA, 20 nt, synthetic construct
  source 1..20: mol_type = other RNA
ggcctctgattagggtgggg

SEQ ID NO: 52 - RNA, 20 nt, synthetic construct
  source 1..20: mol_type = other RNA
gcctctgattagggtggggg

SEQ ID NO: 53 - RNA, 20 nt, synthetic construct
  source 1..20: mol_type = other RNA
taccccacccacgcccccac

SEQ ID NO: 54 - RNA, 20 nt, synthetic construct
  source 1..20: mol_type = other RNA
gaggccaaacccttcctgga

SEQ ID NO: 55 - RNA, 20 nt, synthetic construct
  source 1..20: mol_type = other RNA
ctggagcctgtgataaaagc

SEQ ID NO: 56 - RNA, 20 nt, synthetic construct
  source 1..20: mol_type = other RNA
agcctgtgataaaagcaact

SEQ ID NO: 57 - RNA, 20 nt, synthetic construct
  source 1..20: mol_type = other RNA
gataaaagcaactgttagct

SEQ ID NO: 58 - RNA, 20 nt, synthetic construct
  source 1..20: mol_type = other RNA
taaaagcaactgttagcttg

SEQ ID NO: 59 - RNA, 20 nt, synthetic construct
  source 1..20: mol_type = other RNA
gcttgcactagactagcttc

SEQ ID NO: 60 - RNA, 20 nt, synthetic construct
  source 1..20: mol_type = other RNA
cactagactagcttcaaagt

SEQ ID NO: 61 - RNA, 20 nt, synthetic construct
  source 1..20: mol_type = other RNA
aaagttgtattgaccctggt

SEQ ID NO: 62 - RNA, 20 nt, synthetic construct
  source 1..20: mol_type = other RNA
aagttgtattgaccctggtg

SEQ ID NO: 63 - RNA, 20 nt, synthetic construct
  source 1..20: mol_type = other RNA
tattgaccctggtgtgttat

SEQ ID NO: 64 - RNA, 20 nt, synthetic construct
  source 1..20: mol_type = other RNA
attgaccctggtgtgttatg

SEQ ID NO: 65 - RNA, 20 nt, synthetic construct
  source 1..20: mol_type = other RNA
accctggtgtgttatgtcta

SEQ ID NO: 66 - RNA, 20 nt, synthetic construct
  source 1..20: mol_type = other RNA
gacataacacaccagggtca

SEQ ID NO: 67 - RNA, 20 nt, synthetic construct
  source 1..20: mol_type = other RNA
atacaactttgaagctagtc

SEQ ID NO: 68 - RNA, 20 nt, synthetic construct
  source 1..20: mol_type = other RNA
ggtggggctgctccaggcat

SEQ ID NO: 69 - RNA, 20 nt, synthetic construct
  source 1..20: mol_type = other RNA
tccaggcatgcagatcccac

SEQ ID NO: 70 - RNA, 20 nt, synthetic construct
  source 1..20: mol_type = other RNA
agatcccacaggcgccctgg

SEQ ID NO: 71 - RNA, 20 nt, synthetic construct
  source 1..20: mol_type = other RNA
cacaggcgccctggccagtc

SEQ ID NO: 72 - RNA, 20 nt, synthetic construct
  source 1..20: mol_type = other RNA
ccagtcgtctgggcggtgct

SEQ ID NO: 73 - RNA, 20 nt, synthetic construct
  source 1..20: mol_type = other RNA
tctgggcggtgctacaactg

SEQ ID NO: 74 - RNA, 20 nt, synthetic construct
  source 1..20: mol_type = other RNA
gggcggtgctacaactgggc

SEQ ID NO: 75 - RNA, 20 nt, synthetic construct
  source 1..20: mol_type = other RNA
tacaactgggctggcggcca

SEQ ID NO: 76 - RNA, 20 nt, synthetic construct
  source 1..20: mol_type = other RNA
gctggcggccaggatggttc

SEQ ID NO: 77 - RNA, 20 nt, synthetic construct
  source 1..20: mol_type = other RNA
ccgccagcccagttgtagca

SEQ ID NO: 78 - RNA, 20 nt, synthetic construct
  source 1..20: mol_type = other RNA
tagcaccgcccagacgactg

SEQ ID NO: 79 - RNA, 20 nt, synthetic construct
  source 1..20: mol_type = other RNA
ccagggcgcctgtgggatct

SEQ ID NO: 80 - RNA, 20 nt, synthetic construct
  source 1..20: mol_type = other RNA
tcccacagatatccagaacc

SEQ ID NO: 81 - RNA, 20 nt, synthetic construct
  source 1..20: mol_type = other RNA
cacagatatccagaaccctg

SEQ ID NO: 82 - RNA, 20 nt, synthetic construct
  source 1..20: mol_type = other RNA
agaaccctgaccctgccgtg

SEQ ID NO: 83 - RNA, 20 nt, synthetic construct
  source 1..20: mol_type = other RNA
ccctgccgtgtaccagctga

SEQ ID NO: 84 - RNA, 20 nt, synthetic construct
  source 1..20: mol_type = other RNA
cgtgtaccagctgagagact

SEQ ID NO: 85 - RNA, 20 nt, synthetic construct
  source 1..20: mol_type = other RNA
accagctgagagactctaaa

SEQ ID NO: 86 - RNA, 20 nt, synthetic construct
  source 1..20: mol_type = other RNA
gagactctaaatccagtgac

SEQ ID NO: 87 - RNA, 20 nt, synthetic construct
  source 1..20: mol_type = other RNA
accgattttgattctcaaac

SEQ ID NO: 88 - RNA, 20 nt, synthetic construct
  source 1..20: mol_type = other RNA
attctcaaacaaatgtgtca

SEQ ID NO: 89 - RNA, 20 nt, synthetic construct
  source 1..20: mol_type = other RNA
tcaaacaaatgtgtcacaaa

SEQ ID NO: 90 - RNA, 20 nt, synthetic construct
  source 1..20: mol_type = other RNA
tgatgtgtatatcacagaca

SEQ ID NO: 91 - RNA, 20 nt, synthetic construct
  source 1..20: mol_type = other RNA
tctgtgatatacacatcaga

SEQ ID NO: 92 - RNA, 20 nt, synthetic construct
  source 1..20: mol_type = other RNA
ctttgtgacacatttgtttg

SEQ ID NO: 93 - RNA, 20 nt, synthetic construct
  source 1..20: mol_type = other RNA
tgacacatttgtttgagaat

SEQ ID NO: 94 - RNA, 20 nt, synthetic construct
  source 1..20: mol_type = other RNA
tttgagaatcaaaatcggtg

SEQ ID NO: 95 - RNA, 20 nt, synthetic construct
  source 1..20: mol_type = other RNA
agaatcaaaatcggtgaata

SEQ ID NO: 96 - RNA, 20 nt, synthetic construct
  source 1..20: mol_type = other RNA
tcactggatttagagtctct

SEQ ID NO: 97 - RNA, 20 nt, synthetic construct
  source 1..20: mol_type = other RNA
agagtctctcagctggtaca

SEQ ID NO: 98 - RNA, 20 nt, synthetic construct
  source 1..20: mol_type = other RNA
gagtctctcagctggtacac

SEQ ID NO: 99 - RNA, 20 nt, synthetic construct
  source 1..20: mol_type = other RNA
ctcagctggtacacggcagg

SEQ ID NO: 100 - RNA, 20 nt, synthetic construct
  source 1..20: mol_type = other RNA
cagctggtacacggcagggt

SEQ ID NO: 101 - RNA, 20 nt, synthetic construct
  source 1..20: mol_type = other RNA
tacacggcagggtcagggtt

SEQ ID NO: 102 - RNA, 20 nt, synthetic construct
  source 1..20: mol_type = other RNA
gggttctggatatctgtggg

SEQ ID NO: 103 - RNA, 20 nt, synthetic construct
  source 1..20: mol_type = other RNA
tggatatctgtgggacaaga

SEQ ID NO: 104 - RNA, 38 nt, synthetic construct
  source 1..38: mol_type = other RNA
  misc_feature 37: note = The nucleotide base "n" can be repeated one to twelve times
  misc_feature 38: note = The nucleotide base "u" can be repeated one to eight times or may be absent
caacgattgcccctcacgaggggacnnnnnnnnnnnnt

SEQ ID NO: 105 - RNA, 49 nt, synthetic construct
  source 1..49: mol_type = other RNA
  misc_feature 48: note = The nucleotide base "n" can be repeated one to twelve times
  misc_feature 49: note = The nucleotide base "u" can be repeated one to eight times or may be absent
gtcggaacgctcaacgattgcccctcacgaggggacnnnnnnnnnnnnt

SEQ ID NO: 106 - RNA, 39 nt, synthetic construct
  source 1..39: mol_type = other RNA
  misc_feature 38: note = The nucleotide base "n" can be repeated one to twelve times
  misc_feature 39: note = The nucleotide base "u" can be repeated one to eight times or may be absent
gcaacgattgcccctcacgaggggacnnnnnnnnnnnnt

SEQ ID NO: 107 - RNA, 50 nt, synthetic construct
  source 1..50: mol_type = other RNA
  misc_feature 49: note = The nucleotide base "n" can be repeated one to twelve times
  misc_feature 50: note = The nucleotide base "u" can be repeated one to eight times or may be absent
ggtcggaacgctcaacgattgcccctcacgaggggacnnnnnnnnnnnnt

SEQ ID NO: 108 - RNA, 40 nt, synthetic construct
  source 1..40: mol_type = other RNA
  misc_feature 39: note = The nucleotide base "n" can be repeated one to twelve times
  misc_feature 40: note = The nucleotide base "u" can be repeated one to eight times or may be absent
ggcaacgattgcccctcacgaggggacnnnnnnnnnnnnt

SEQ ID NO: 109 - RNA, 51 nt, synthetic construct
  source 1..51: mol_type = other RNA
  misc_feature 50: note = The nucleotide base "n" can be repeated one to twelve times
  misc_feature 51: note = The nucleotide base "u" can be repeated one to eight times or may be absent
gggtcggaacgctcaacgattgcccctcacgaggggacnnnnnnnnnnnnt

SEQ ID NO: 110 - DNA, 22 bp, synthetic construct
  source 1..22: mol_type = other DNA
gctggacttcagcccaaaatgc

SEQ ID NO: 111 - DNA, 22 bp, synthetic construct
  source 1..22: mol_type = other DNA
agtccaacccagtgttcctctc

SEQ ID NO: 112 - DNA, 22 bp, synthetic construct
  source 1..22: mol_type = other DNA
gagacttcctgcctcgtcacaa

SEQ ID NO: 113 - DNA, 22 bp, synthetic construct
  source 1..22: mol_type = other DNA
gatcagcaggaaaacacagccg

SEQ ID NO: 114 - RNA, 25 nt, synthetic construct
  source 1..25: mol_type = other RNA
caacgattgcccctcacgaggggac

SEQ ID NO: 115 - RNA, 36 nt, synthetic construct
  source 1..36: mol_type = other RNA
gtcggaacgctcaacgattgcccctcacgaggggac

(19) United States
(12) Patent Application Publication
(10) Pub. No.: US 2026/0146241 A1
(43) Pub. Date: May 28, 2026

(54) Engineered CasPhi2 Nucleases

(71) Applicant: The General Hospital Corporation, Boston, MA (US)

(72) Inventors: J. Keith Joung, Winchester, MA (US); Julian Grünewald, Munich (DE); Bret Miller, Niceville, FL (US); Eliza Jane Holtz, Salem, MA (US); SeHee Park, Charlestown, MA (US)

(21) Appl. No.: 19/122,643

(22) PCT Filed: Oct. 23, 2023
(86) PCT No.: PCT/US2023/077523

§ 371 Date: Apr. 18, 2025

Related U.S. Application Data
(60) Provisional application No. 63/418,359, filed on Oct. 21, 2022.

(51) Int. Cl. C12N 9/22; C07K 14/47; C12N 9/02; C12N 9/10; C12N 9/78; C12N 9/80; C12N 15/11; C12N 15/90

(52) U.S. Cl. CPC C12N 9/226; C07K 14/4703; C12N 9/0071; C12N 9/1007; C12N 9/1029; C12N 9/78; C12N 9/80; C12N 15/11; C12N 15/907; C12Y 203/01048; C12Y 305/01098; C12Y 305/04004; C12Y 305/04005; C07K 2319/00; C12N 2310/20; C12N 2320/10

(57) ABSTRACT

Described herein are variants of CasPhi2 nucleases with enhanced editing capabilities and methods of use thereof.

### CROSS-REFERENCE TO RELATED APPLICATIONS

This application claims the benefit of priority to U.S. Application No. 63/418,359, filed on Oct. 21, 2022, the contents of which are hereby incorporated by reference.

### FEDERALLY SPONSORED RESEARCH OR DEVELOPMENT

This invention was made with Government support under Grant Nos. R35 GM118158 and RM1 HG009490 awarded by the National Institutes of Health. The Government has certain rights in the invention.

### TECHNICAL FIELD

The present disclosure provides CasPhi2 polypeptides that exhibit enhanced gene editing cleavage activity, compared to a wild-type CasPhi2 polypeptide. The present disclosure provides systems, methods, and kits comprising such CasPhi2 polypeptides.

### BACKGROUND

CRISPR (clustered regularly interspaced short palindromic repeats) systems, which can be found in bacteria and archaea, have transformed the field of gene editing due to their robust and facile DNA targeting capabilities. RNA-guided CRISPR-associated (Cas) nucleases can induce targeted DNA double-strand breaks (DSBs) and thereby induce highly efficient edits via non-homologous end-joining (NHEJ) or homology-directed repair (HDR)^{1,2}. The most commonly used Cas proteins for gene editing in human cells are the Cas9 and Cas12a nucleases³. One limitation of these nucleases is their relatively large sizes—for example, the widely used SpCas9 and LbCas12a enzymes are 1368 and 1228 amino acids in length, respectively-which can create issues for encoding these enzymes in size-constrained viral vectors (e.g., adeno-associated viruses) and for production and manufacturing of these proteins or RNAs encoding them. This large size becomes even more pronounced when Cas nickase and/or catalytically inactive versions of these enzymes are fused to other proteins to create next-generation “CRISPR 2.0” editors such as base editors, prime editors, or epigenetic editors^{4,5}.

Mining of varied bacterial and bacteriophage genomes has yielded new “hypercompact” Cas proteins that are substantially smaller in size than the larger Cas9, Cas12a, and Cas12i^{6,7} enzymes but these substantially smaller size proteins generally all have certain limitations that make them less optimal for use in human cells. For example, recent work on Cas12f (Cas14⁸) proteins like Acidibacillus sulfuroxidans Cas12fl (AsCas12f1, 422 aa)⁹ or engineered CasMINI (529 aa)¹⁰ (based on a Cas12f from uncultivated archaea¹¹) function as nucleases in human cells and induce only modest indel frequencies in human cells ranging from ˜10% 10 to ˜33% 9. Catalytically inactive versions of these Cas12f (Cas14) proteins do function efficiently as targetable epigenetic editors in human cells when fused to transcriptional activation domains¹⁰. However, Cas12f has been shown to function as an “asymmetric homodimer”, which might limit its utility¹², and Cas12f proteins have longer length or more complex PAM sequences (e.g., 5′TTTR^{10,11} or 5′NTTR, 5′-CTCA and 5′-TTCA⁹) that also restrict their targeting range. Transposon-associated TnpB, a probable phylogenetic ancestor of the Cas12 family, has been used as a hypercompact (557 aa) programmable RNA-guided nuclease and base editor as well, yielding up to ˜60% nuclease-induced indel frequencies in human cells¹³ and up to ˜40% ABE activity when fused to adenosine deaminases¹⁴. However, current TnpB editors also possess a lengthy PAM (5′-TTTR or 5′-TTTN)¹³ that again limits its targeting range. Recent work has also described the identification of CRISPR-CasΦ nucleases from bacteriophages (type V-J, Cas12j-2) that are only ˜700-800 amino acids in length¹⁵, approximately half the size of the SpCas9 nuclease. Initial characterization of the CasPhi2 enzyme suggested that it could induce modest gene editing frequencies as a nuclease in human cells although these activities were measured only indirectly (via loss of expression of a GFP reporter gene) and not by direct measurement of induced mutations (indels) by DNA sequencing¹⁵.

### SUMMARY

Provided herein are engineered isolated CasPhi2 proteins (i.e., CasPhi2 variants) with enhanced editing capabilities and methods of use thereof.

In a first aspect, the invention provides isolated CasPhi2 proteins, comprising an amino acid sequence having at least 70%, 75%, 80%, 85%, 90%, or 95% sequence identity to the amino acid sequence of SEQ ID NO:1, and comprising a mutation at one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more, thirty-nine or more, forty or more, forty-one or more, forty-two or more, forty-three or more, forty-four or more, forty-five or more, forty-six or more, forty-seven or more, forty-eight or more, forty-nine or more, fifty or more, fifty-one or more, fifty-two or more, fifty-three or more, fifty-four or more, fifty-five or more, fifty-six or more, fifty-seven or more, or all of the following positions: S11, S25, A36, S106, E107, S124, D134, G138, L149, A156, E159, S160, S164, D167, E168, T203, P233, D337, A261, P277, T355, T357, L370, D427, D428, A435, N497, L506, S507, N508, S509, S511, D513, Q514, T518, P519, A520, P521, G524, A525, K526, K527, P530, V531, E532, V533, R538, T539, E569, L571, S574, E578, S616, T628, T649, D679, Q684, and/or T691.

In a second aspect, the invention provides isolated CasPhi2 proteins, comprising an amino acid sequence having at least 70%, 75%, 80%, 85%, 90%, or 95% sequence identity to the amino acid sequence of SEQ ID NO: 1, and comprising a mutation at one or more of the following positions: T355 and/or D679. In some embodiments, the isolated CasPhi2 protein further comprises a mutation at one or more of the following positions: S11, S25, A36, S106, E107, S124, D134, G138, L149, A156, E159, S160, S164, D167, E168, T203, P233, A261, P277, D337, T357, L370, D427, D428, A435, N497, L506, S507, N508, S509, S511, D513, Q514, T518, P519, A520, P521, G524, A525, K526, K527, P530, V531, E532, V533, R538, T539, A543 E569, L571, S574, E578, S616, T628, T649, E674, Q684, and/or T691.

In some embodiments, any of the CasPhi2 proteins described above comprise a mutation at T355 and the mutation is T355R or T355K.

In some embodiments, any of the CasPhi2 proteins described above comprise a mutation at D679 and the mutation is D679R, D679K, D679H, or D679T.

In some embodiments, any of the CasPhi2 proteins described above comprise one of the combinations of mutations listed in Table 1.

In some embodiments, the isolated CasPhi2 protein comprises the following mutations: A36R, S106R, D134R, L149R, E159A, S160A, S164A, D167K, E168A, P277R, T357K, T518R, L571K, S616R, Q684R, T355R, and D679K.

In some embodiments, the isolated CasPhi2 protein comprises the following mutations: A36R, S106R, D134R, P277R, T355R, T357K, T518R, L571K, S616R, D679K, and Q684R. In some embodiments, the isolated CasPhi2 protein further comprises a mutation at one or more of the following positions: S11, S25, G138, T203, A261, D337, N497, L506, S507, N508, S509, D513, Q514, A520, G524, A525, K527, P530, V531, R538, T539, R542, A543, E569, E578, T628, T649, E674, and/or T691. In some embodiments, the isolated CasPhi2 protein further comprises the following mutations: F23S and S26R. In some embodiments, the isolated CasPhi2 protein further comprises the following mutations: T340G, D341R, and D342G.

In some embodiments, the isolated CasPhi2 protein comprises the following mutations: A36R, S106R, D134R, L149R, P277R, T355R, T357K, T518R, L571K, S616R, D679K, and Q684R.

In some embodiments, the isolated CasPhi2 protein comprises the following mutations: A36K, S106K, D134K, P277K, D337K, T355R, T357K, V531R, T539A, A543K, L571K, S616K, D679K, and T691K. In some embodiments, the isolated CasPhi2 protein further comprises the following mutations: further comprises the following mutation: Q684R.

In some embodiments, any of the CasPhi2 proteins described above further comprise a mutation that catalytically inactivates nuclease activity, wherein the mutation is D394A of SEQ ID NO:1.

In some embodiments, any of the CasPhi2 proteins described above further a mutation that catalytically impairs nuclease activity, wherein the mutation is E606Q of SEQ ID NO: 1.

Also provided herein are fusion proteins comprising any of the CasPhi2 proteins described above, fused to at least one heterologous functional domain, with an optional intervening linker, wherein the linker does not interfere with activity of the fusion protein.

In some embodiments, the heterologous functional domain is a transcriptional activation domain. In some embodiments, the transcriptional activation domain is VP16, VP64, Rta, NF-κB p65, p300, or a VPR fusion.

In some embodiments, the heterologous functional domain is a transcriptional silencer or transcriptional repression domain. In some embodiments, the transcriptional repression domain is a Krueppel-associated box (KRAB) domain, ERF repressor domain (ERD), or mSin3A interaction domain (SID). In some embodiments, the transcriptional silencer is Heterochromatin Protein 1 (HP1).

In some embodiments, the heterologous functional domain is an enzyme that modifies the methylation state of DNA. In some embodiments, the enzyme that modifies the methylation state of DNA is a DNA methyltransferase (DNMT) or a TET protein. In some embodiments, the TET protein is TET1.

In some embodiments, the heterologous functional domain is an enzyme that modifies a histone subunit. In some embodiments, the enzyme that modifies a histone subunit is a histone acetyltransferase (HAT), histone deacetylase (HDAC), histone methyltransferase (HMT), or histone demethylase.

In some embodiments, the heterologous functional domain is a biological tether. In some embodiments, the biological tether is MS2, Csy4 or lambda N protein.

In some embodiments, the heterologous functional domain is FokI.

In some embodiments, the heterologous functional domain is a deaminase. In some embodiments, the heterologous functional domain is a cytidine deaminase. In some embodiments, the cytidine deaminase is selected from the group consisting of APOBEC1, APOBEC2, APOBEC3A, APOBEC3B, APOBEC3C, APOBEC3D/E, APOBEC3F, APOBEC3G, APOBEC3H, APOBEC4, activation-induced cytidine deaminase (AID), cytosine deaminase 1 (CDA1), pmCDA1, CDA2, and cytosine deaminase acting on tRNA (CDAT). In some embodiments, the heterologous functional domain is an adenosine deaminase. In some embodiments, the adenosine deaminase is selected from the group consisting of adenosine deaminase 1 (ADA1), ADA2; adenosine deaminase acting on RNA 1 (ADAR1), ADAR2, ADAR3; adenosine deaminase acting on tRNA 1 (ADAT1), ADAT2, ADAT3; and naturally occurring or engineered tRNA-specific adenosine deaminase (TadA).

In some embodiments, the fusion protein comprises at least two heterologous functional domains, wherein the additional heterologous functional domain comprises an enzyme, domain, or peptide that inhibits or enhances endogenous DNA repair or base excision repair (BER) pathways. In some embodiments, the additional heterologous functional domain is a uracil DNA glycosylase inhibitor (UGI) that inhibits uracil DNA glycosylase (UDG, also known as uracil N-glycosylase, or UNG); or Gam from the bacteriophage Mu.

Also provided herein are isolated nucleic acids encoding any of the isolated CasPhi2 proteins described above or any of the fusion proteins described above. Also provided herein are the vectors comprising the isolated nucleic acids. Also provided herein are host cells, e.g., mammalian host cells, comprising the nucleic acids described herein, and optionally expressing any of the isolated CasPhi2 proteins described above or any of the fusion proteins described above.

In another aspect, also provided herein are compositions comprising: an isolated nucleic acid encoding any of the isolated CasPhi2 proteins described above or any of the fusion proteins described above; and a nucleic acid comprising or encoding one or more crRNAs or pre-crRNAs, optionally an array of two or more pre-crRNAs. In some embodiments, only one crRNA is present. In some embodiments, more than one crRNA is present. In some embodiments, only one pre-crRNA is present. In some embodiments, more than one pre-crRNA is present. In some embodiments, the one or more crRNAs or pre-crRNAs direct the isolated CasPhi2 protein to one or more target genomic sequences. In some embodiments, wherein one or more crRNAs or pre-crRNAs includes a complementarity region that is complementary to 14-24 nucleotides of a respective target genomic sequence or sequences. In some embodiments, wherein the one or more crRNAs or pre-crRNAs comprises the following sequence:
5′-CAACGAUUGCCCCUCACGAGGGGAC-N₁₂₋₂₄-U₀₋₈, SEQ ID NO: 104,
5′-GUCGGAACGCUCAACGAUUGCCCCUCACGAGGGGAC-N₁₂₋₂₄-U₀₋₈, SEQ ID NO: 105,
5′-GCAACGAUUGCCCCUCACGAGGGGAC-N₁₂₋₂₄-U₀₋₈, SEQ ID NO: 106,
5′-GGUCGGAACGCUCAACGAUUGCCCCUCACGAGGGGAC-N₁₂₋₂₄-U₀₋₈, SEQ ID NO: 107,
5′-GGCAACGAUUGCCCCUCACGAGGGGAC-N₁₂₋₂₄-U₀₋₈, SEQ ID NO: 108, or
5′-GGGUCGGAACGCUCAACGAUUGCCCCUCACGAGGGGAC-N₁₂₋₂₄-U₀₋₈, SEQ-ID No. 109, and wherein N is any nucleotide, and wherein the one or more crRNAs or pre-crRNAs is designed to be complementary to the respective target genomic sequence or sequences.

Also provided herein are methods of altering a genome of a cell, the method comprising expressing in the cell, or contacting the cell with, any of the isolated CasPhi2 proteins described above or any of the fusion proteins described above, and one or more crRNAs or pre-crRNAs or a nucleic acid comprising or encoding one or more crRNAs or pre-crRNAs, optionally an array of two or more pre-crRNAs, wherein the one or more crRNAs or pre-crRNAs direct the isolated CasPhi2 protein described above or any of the fusion proteins described above to one or more target genomic sequences. In some embodiments, only one crRNA is present. In some embodiments, more than one crRNA is present. In some embodiments, only one pre-crRNA is present. In some embodiments, more than one pre-crRNA is present. In some embodiments, the cell is a stem cell. In some embodiments, the stem cell is an embryonic stem cell, a mesenchymal stem cell, or an induced pluripotent stem cell; is in a living animal; or is in or is an embryo.

Also provided herein are methods of altering a double stranded DNA (dsDNA) molecule, the method comprising contacting the dsDNA with any of the isolated CasPhi2 proteins described above or any of the fusion proteins described above, and one or more crRNAs or pre-crRNAs or a nucleic acid comprising or encoding one or more crRNAs or pre-crRNAs, optionally an array of two or more pre-crRNAs, wherein the one or more crRNAs or pre-crRNAs direct the isolated CasPhi2 protein described above or any of the fusion proteins described above to one or more target genomic sequences. In some embodiments, only one crRNA is present. In some embodiments, more than one crRNA is present. In some embodiments, only one pre-crRNA is present. In some embodiments, more than one pre-crRNA is present. In some embodiments, the dsDNA molecule is in vitro.

In some embodiments of any of the methods described above, wherein the one or more crRNAs or pre-crRNAs includes a complementarity region that is complementary to 14-24 nucleotides of the one or more target genomic sequences. In some embodiments, the one or more crRNAs or pre-crRNAs comprises the following sequence:
5′-CAACGAUUGCCCCUCACGAGGGGAC-N₁₂₋₂₄-U₀₋₈, SEQ ID NO: 104,
5′-GUCGGAACGCUCAACGAUUGCCCCUCACGAGGGGAC-N₁₂₋₂₄-U₀₋₈, SEQ ID NO: 105,
5′-GCAACGAUUGCCCCUCACGAGGGGAC-N₁₂₋₂₄-U₀₋₈, SEQ ID NO: 106,
5′-GGUCGGAACGCUCAACGAUUGCCCCUCACGAGGGGAC-N₁₂₋₂₄-U₀₋₈, SEQ ID NO: 107,
5′-GGCAACGAUUGCCCCUCACGAGGGGAC-N₁₂₋₂₄-U₀₋₈, SEQ ID NO: 108, or
5′-GGGUCGGAACGCUCAACGAUUGCCCCUCACGAGGGGAC-N₁₂₋₂₄-U₀₋₈, SEQ-ID No. 109, and wherein N is any nucleotide, and wherein the one or more crRNAs or pre-crRNAs is designed to be complementary to the respective target genomic sequence or sequences.

In some embodiments, of any of the methods described above, further comprising co-expressing and/or contacting an additional single- or double-stranded DNA donor (ssODN or dsODN) in the cell to enable homologous recombination or homology-directed repair with that ssODN or dsODN donor to introduce alterations, deletions, or insertions in the proximity of the site of the double-stranded break induced by any of the isolated CasPhi2 protein described above or any of the fusion proteins described above.

Also provided herein are kits comprising: (a) any of the isolated CasPhi2 proteins described above or any of the fusion proteins described above, or nucleic acids encoding any of the isolated CasPhi2 proteins described above or any of the fusion proteins described above; (b) one or more crRNAs or pre-crRNAs comprising one or more of the following sequences:
5′-CAACGAUUGCCCCUCACGAGGGGAC-N₁₂₋₂₄-U₀₋₈, SEQ ID NO: 104,
5′-GUCGGAACGCUCAACGAUUGCCCCUCACGAGGGGAC-N₁₂₋₂₄-U₀₋₈, SEQ ID NO: 105,
5′-GCAACGAUUGCCCCUCACGAGGGGAC-N₁₂₋₂₄-U₀₋₈, SEQ ID NO: 106,
5′-GGUCGGAACGCUCAACGAUUGCCCCUCACGAGGGGAC-N₁₂₋₂₄-U₀₋₈, SEQ ID NO: 107,
5′-GGCAACGAUUGCCCCUCACGAGGGGAC-N₁₂₋₂₄-U₀₋₈, SEQ ID NO: 108, or
5′-GGGUCGGAACGCUCAACGAUUGCCCCUCACGAGGGGAC-N₁₂₋₂₄-U₀₋₈, SEQ-ID No. 109, and wherein N is any nucleotide, and wherein the one or more crRNAs or pre-crRNAs is designed to be complementary to the respective target genomic sequence or sequences, or nucleic acids encoding the one or more crRNAs or pre-crRNAs; and (c) a single-stranded DNA with a signal detectable upon cleavage.

Also provided herein are methods of detecting a target DNA sequence in vitro, the method comprising: incubating a DNA sample with: (a) any of the isolated CasPhi2 proteins described above or any of the fusion proteins described above; (b) one or more crRNAs or pre-crRNAs comprising one or more of the following sequences:
5′-CAACGAUUGCCCCUCACGAGGGGAC-N₁₂₋₂₄-U₀₋₈, SEQ ID NO: 104,
5′-GUCGGAACGCUCAACGAUUGCCCCUCACGAGGGGAC-N₁₂₋₂₄-U₀₋₈, SEQ ID NO: 105,
5′-GCAACGAUUGCCCCUCACGAGGGGAC-N₁₂₋₂₄-U₀₋₈, SEQ ID NO: 106,
5′-GGUCGGAACGCUCAACGAUUGCCCCUCACGAGGGGAC-N₁₂₋₂₄-U₀₋₈, SEQ ID NO: 107,
5′-GGCAACGAUUGCCCCUCACGAGGGGAC-N₁₂₋₂₄-U₀₋₈, SEQ ID NO: 108, or
5′-GGGUCGGAACGCUCAACGAUUGCCCCUCACGAGGGGAC-N₁₂₋₂₄-U₀₋₈, SEQ-ID No. 109, and wherein N is any nucleotide, and wherein the one or more crRNAs or pre-crRNAs is designed to be complementary to the respective target genomic sequence or sequences; and (c) a single-stranded DNA with a detectable signal upon cleavage, and determining the presence or absence of the detectable signal. In some embodiments, two or more crRNAs designed to recognize two or more target DNA sequences are provided as pre-crRNAs encoded in a single array that are then processed into individual crRNAs by any of the isolated CasPhi2 proteins described above or any of the fusion proteins described above.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1F. WT CasPhi2 exhibits non-robust and inefficient gene editing activity in human cells. (A) Testing of WT CasPhi2 with previously described crRNAs (crRNA 6 or crRNA 8) that target GFP coding sequence in HEK293-GFP reporter cells harboring an integrated GFP gene. Additional negative and positive controls shown were also tested side-by-side. Percentages of GFP-negative cells as measured by flow cytometry are shown for each condition (n=3, independent replicates). (B) Dot and bar plots showing indel frequencies (y-axis) induced by WT CasPhi2 with crRNA 6 or crRNA 8 at their respective targeted GFP sites in HEK293-GFP reporter cells as determined by targeted amplicon sequencing using next-generation sequencing (NGS) (n=3, independent replicates). (C) Dot and bar plots showing indel frequencies (y-axis) induced by WT CasPhi2 with 19 different individual crRNAs each targeting endogenous genomic loci in human HEK293T cells as determined by targeted amplicon sequencing of each intended on-target site using NGS. (n=3, independent replicates). Negative controls were untreated cells, seeded in parallel (“no treatment”). (D) Schematic map showing pUC19-based U6 entry expression vector (right side of figure) and DNA sequences for expressing CasPhi2 pre-crRNAs and crRNAs, including pre-crRNA and crRNA architecture delineating direct repeat lengths used (left side of figure). (E) Dot and bar plots showing indel frequencies (y-axis) induced by WT CasPhi2 with 17 different individual pre-crRNAs each targeting endogenous genomic loci in human HEK293T cells as determined by targeted amplicon sequencing of each intended on-target site using NGS (n=3, independent replicates). Negative controls were cells co-transfected with plasmids expressing catalytically inactive dWTCasPhi2 (D394A) and each of the respective pre-crRNAs. (F) Allele DNA sequences and their frequencies from targeted amplicon sequencing experiments from (E) for the VEGFA site 3 pre-crRNA with either a negative control (dWTCasPhi2 (D394A)) (left) or WT CasPhi2 nuclease (right). Note the insertion/deletion (indel) profile induced by WT CasPhi2 in human cells, i.e. predominantly deletions between 2 bp and >40 bp in length (often ˜4-8 bp) and insertions of various sizes (1-15 bp) at much lower frequencies.

FIGS. 2A-2K. Engineering of CasPhi2 variants with increased gene editing activities in human cells-STAGE I (A) Amino acid sequence alignments of WT CasPhi2 with Cas12f (aka Cas14), the most closely related prokaryotic CRISPR system. Note the relatively low amino acid (AA) homology across the entire protein as well as across the catalytic RuvC domain (upper panel). Expanded and more detailed view of the amino acid sequences of the REC dimerization and PAM interaction domains shows homology between these proteins at a small number of residues (lower panel). (B) Schematic illustrating the subset of CasPhi2 residues of interest for Stage I engineering and potential AA mutations based on the homology studies with Cas12f and the available Cas12f structure. (C) Dot and bar plots showing indel frequencies (y-axis) induced by 20 different CasPhi2 variants that were designed during Stage I engineering and each tested with a single crRNA targeting the VEGFA site 3 in human HEK293T cells as determined by targeted amplicon sequencing of this site using NGS (n=3, independent replicates). CasPhi2 variants are labeled as “CasPhiX ###Y” where X is the original amino acid present at position ###and Y is the mutated amino acid present in the variant. Note that this initial screening yielded two CasPhi2 variants that induced substantially increased indel frequencies: T355R and D679K. Dotted line indicates indel frequencies induced by WT CasPhi2 (n=3, independent replicates). (D) Dot and bar plots showing indel frequencies (y-axis) induced by dead WT CasPhi2 (D394A) (labeled as WT dCasPhi2 in this figure panel), WT CasPhi2, as well as CasPhi2 variants CasPhi2-T355R and CasPhi2-D679K tested with six crRNAs targeting endogenous loci in human HEK293T cells as determined by targeted amplicon sequencing of each on-target site using NGS (n=3, independent replicates). (E) Dot and bar plots showing indel frequencies (y-axis) induced by dead WT CasPhi2 (labeled as WT dCasPhi2 in this figure panel), WT CasPhi2, CasPhi2 variants CasPhi2-T355R and CasPhi2-D679K, and the combination variant (the “double-mutant” CasPhi2-DM (harboring both the T355R and D679K mutations)) tested with four crRNAs targeting endogenous loci in human HEK293T cells as determined by targeted amplicon sequencing of each on-target site using NGS (n=3, independent replicates). (F) Dot and bar plots showing indel frequencies (y-axis) induced by “no treatment” negative control, WT CasPhi2, and CasPhi2-DM (T355R-D679K) side-by-side, tested with 27 crRNAs targeting endogenous loci in human HEK293T cells as determined by targeted amplicon sequencing of each on-target site using NGS (n=3, independent replicates). (G) Dot and bar plots showing indel frequencies (y-axis) induced by “no treatment” negative control, WT CasPhi2, and CasPhi2-DM (T355R-D679K) (the latter encoded using a different codon optimization (GenScript optimum)) tested with four crRNAs targeting endogenous loci in human induced pluripotent stem cell-derived cardiomyocytes (hiPSC-CMs) as determined by targeted amplicon sequencing of each on-target site using NGS (n=3, independent replicates). (H) Dot and bar plots showing indel frequencies (y-axis) induced by CasPhi2-DM (T355R-D679K) tested with 12 or 24 crRNAs tiled across four different endogenous genomic loci of potential clinical interest in human HEK293T cells as determined by targeted amplicon sequencing of each on-target site using NGS (n=3, independent replicates). (I) Heat maps indicating A-to-G adenine base editing frequencies across all adenines of the on-target spacers ofvarious endogenous human gene loci (targeted with a crRNA) using ABE fusions comprising catalytically inactive (i.e. “dead”) dWT CasPhi2 (with a D394A active site mutation) or dCasPhi2-DM (with a D394A mutation) fused to the TadA8e adenine deaminase, compared to no treatment controls. For the dCasPhi2-DM based fusions, TadA8e was fused to the N-terminal end of C-terminal end of dCasPhi2-DM. In this figure, dCasPhi2-DM is labeled as “dCasPhi2 (DM)” in the table labels. Data shown from experiments in which eight crRNAs targeting endogenous genomic loci were tested in HEK293T cells. Editing frequencies were determined by targeted amplicon sequencing of each on-target site spacer using NGS (n=3, independent replicates). (J) Gene activating activities of dWT-CasPhi2 and dCasPhi2-DM fusions with the synthetic VPR activation domain with single or pooled crRNAs targeting the promoter regions of CD69 and IL2RA genes in HEK293T cells (n=1). Fold-activation values were determined by calculating the level of mRNA expression of the target gene as measured by quantitative RT-PCR in the presence of the targeted crRNA(s) over that in the presence of a non-targeting crRNA. “VPR-CasPhi2_DM (N-term)” and “CasPhi2_DM-VPR (C-term)” indicate fusions of VPR to the N-terminus and C-terminus, respectively, of dCasPhi2-DM. “WT_CasPhi2-VPR (C-term)” indicates a fusion of VPR to the C-terminus of dWT CasPhi2. (K) Tables showing the indel frequencies (Indel (%), left table) and fold-increase in indel frequencies relative to WT CasPhi2 (Fold-change, right table) induced by dWT CasPhi2 (labeled as “dCasPhi2” in the table), WT CasPhi2 (labeled as “CasPhi2” in the table), various CasPhi2 variants harboring various amino acid substitutions at positions T355 and D679, and the CasPhi2-DM variant (labeled as “CasPhi2-T355R-D679K” in the table). Indel frequencies or fold-increases relative to WT CasPhi2 are shown for four different crRNAs targeted to various human endogenous gene targets with the mean fold-increase across the four crRNAs shown in the far right column of the table on the right side of the figure. Experiments were performed in HEK293T cells in triplicate with mean indel frequencies shown. Indel frequencies were determined by targeted amplicon sequencing of each on-target site using NGS.

FIGS. 3A-3C. Testing CasPhi2-DM with crRNAs harboring various spacer lengths and for multiplex gene editing with arrays of pre-crRNAs (A) Dot and bar plots showing indel frequencies (y-axes) induced with WT CasPhi2 or CasPhi2-DM tested with crRNAs that have systematically varied spacer lengths at their 3′ end ranging from 12-24 nucleotides (nt) of complementarity to endogenous genomic loci in the VEGFA gene and at matched site 8 in HEK293T cells as determined by targeted amplicon sequencing of each on-target site using NGS (n=2 or n=3, independent replicates). (B) Schematic showing DNA sequences encoding a single pre-crRNA array with multiple direct repeats and three spacers targeting three genomic loci to enable CasPhi2 multiplex gene editing in human cells. Pre-crRNA arrays have been previously shown to be processed and cleaved into individual crRNAs by WT CasPhi2. (C) Dot and bar plots showing indel frequencies (y-axes) induced with WT CasPhi2 or CasPhi2-DM tested with three pre-crRNAs each targeting a single genomic locus (VEGFA site 3, Matched site 8, or FANCF site 1) or with pre-crRNA arrays encoding spacers that can target two or three of these same genomic loci from a single array when expressed in HEK293T cells as determined by targeted amplicon sequencing of each on-target site using NGS (n=2 or n=3, independent replicates).

FIG. 4. Testing the effects of adding previously described CasPhi2 “nickase” and “velocity” variants¹⁶ to the CasPhi2-DM variant. Dot and bar plots showing indel frequencies (y-axes) induced by no treatment controls, WT CasPhi2, the CasPhi2 velocity variant (labeled as “Pausch velocity variant”¹⁶, the CasPhi2 nicking variant (labeled as “Pausch nicking variant” 16), CasPhi2-DM, and combinations thereof as labeled, tested with six crRNAs targeting endogenous genomic loci in human HEK293T cells as determined by targeted amplicon sequencing of each target site using NGS (n=3, independent replicates).

FIGS. 5A-5E. Engineering of CasPhi2 variants with increased gene editing activities in human cells-STAGES II and III (A) Heat maps showing indel frequencies induced by 170 CasPhi2 structure-based variants with four different crRNAs targeting various endogenous human loci in HEK293T cells (Stage II engineering). Each variant has the CasPhi2-DM mutations T355R-D679K and one additional amino acid substitution as labeled in the table. Indel frequencies induced by CasPhi2-DM and in a no-treatment negative control are also shown for all four crRNAs. White-to-grey gradients indicate indel frequencies and are shown in the lower left corner for each of the four target sites. Indel frequencies were determined by targeted amplicon sequencing of each on-target site using NGS. X indicates a sample that was dropped due to low NGS read count (n=1, except for no treatment and CasPhi2-DM, n=4. For these experiments, we show averaged values in the heatmap.). (B) Dot and bar plots showing indel frequencies (y-axes) for a subset of promising variants from (A). Variants are labeled as in (A). These are the same data as shown in (A). Dotted line indicates indel frequencies observed with CasPhi2-DM (labeled as CasPhi2 (T355R-D679K) here). (C) Heat maps showing indel frequencies of new CasPhi2 variants engineered by combining amino acid substitutions from variants shown in (B) that showed higher activities in human cells (Stage III engineering, part 1). All variants shown here harbored the T355R and D679K as well as the additional amino acid substitutions indicated in the figure. Indel frequencies induced by no treatment (labeled as “no_treatment_avg”), WT CasPhi2 (labeled as “CasPhi_WT_avg”), and CasPhi2-DM are shown for comparison. Each of these variants and controls were tested in HEK293T cells with three different crRNAs targeting various endogenous human gene loci (VEGFA s3, matched s5, and EMX1 s1). White-to-grey gradients indicate indel frequencies as determined by targeted amplicon sequencing of each target site using NGS (n=1, except for WT-CasPhi2 and no treatment, n=2. For these experiments, we show averaged values in the heatmap.). (D) Heat maps showing indel frequencies of additional CasPhi2 variants engineered by combining amino acid substitutions from variants shown in (B) and (C) that showed higher activities in human cells (Stage III engineering, part 2). All variants shown here harbored the T355R and D679K as well as the additional amino acid substitutions indicated in the figure. Indel frequencies induced by a “gRNA only” control (labeled as “Negative control”), WT CasPhi2, and CasPhi2-DM are shown for comparison. Each of these variants and controls were tested in HEK293T cells with five different crRNAs targeting various endogenous human gene loci (VEGFA s3, matched s5, EMX1 s1, BCL11A s9, and FANCF s1). White-to-grey gradients indicate indel frequencies as determined by targeted amplicon sequencing of each on-target site using NGS (n=1, except for negative control, WT-CasPhi2, CasPhi2-DM, L149R-D167K-T355R-L571K-D679K (“penta”) and A36R-L149R-D167K-T355R-L571K-S616R-D679K (“hepta”), n=3. For these experiments, we show averaged values in the heatmap.). (E) Heat maps showing indel frequencies of further CasPhi2 variants engineered by combining amino acid substitutions from variants shown in (B), (C), and (D) that showed higher activities in human cells as well as certain individual mutations that were in the “Pausch nickase variant” (Stage III engineering, part 3). All variants shown here (except for the no treatment and the WT CasPhi2 controls) harbored the T355R and D679K DM mutations as well as the additional amino acid substitutions indicated in the figure. Indel frequencies induced by a “gRNA only” control (labeled as “Negative control”), WT CasPhi2, CasPhi2-DM, the Pausch et al CasPhi2 “nickase” variant (bearing five amino acid substitutions E159A, S160A, S164A, D167A, E168A), and a derivative of the Pausch et al CasPhi2 “nickase” variant (in which we replaced the D167A mutation with a D167K mutation we had identified in (A)) are shown for comparison. Each of these variants and controls were tested in HEK293T cells with six different crRNAs targeting various endogenous human gene loci (VEGFA s3, matched s8, EMX1 s1, ABE s2, CD69, and FANCF s1). White-to-grey gradients indicate indel frequencies as determined by targeted amplicon sequencing of each on-target site using NGS (n=1, except for WT-CasPhi2, CasPhi2-DM, A36R-L149R-D167K-P277R-T355R-T357K-L571K-S616R-D679K (“nona”) and E159A-S160A-S164A-D167K-E168A-T355R-D679K (n=3) and the variant containing all amino acid substitutions from the Pausch et al CasPhi2 “nickase” variant, combined with T355R-D679K (n=2). For these experiments, we show averaged values in the heatmap.).

FIGS. 6A-6D. Testing the robustness and gene editing efficiencies of various multiply substituted CasPhi2 variants in human cells. (A) Dot and bar plots showing indel frequencies (y-axes) for seven multiply substituted CasPhi2 (see table in upper left corner) side-by-side with CasPhi2-DM (labeled as “T355R-D679K (DM)” in the table), WT CasPhi2, and a negative control. The seven multiply substituted variants labeled 1-7 in the table all have the T355R and D679K (DM) mutations as well as the additional amino acid substitutions indicated in the table. Note that variant 3 is also referred to here and subsequently as the CasPhi2-17AA variant because it has a total of 17 amino acid substitutions relative to the original wild-type CasPhi2 protein. All CasPhi2 proteins were tested with 32 different crRNAs targeting endogenous genomic loci in HEK293T cells and indel frequencies were determined by targeted amplicon sequencing of each on-target site using NGS, n=3, independent replicates. (B) Dot and bar plots showing indel frequencies (y-axes) induced by CasPhi2-17AA or WT CasPhi2 when tested with 12 or 24 crRNAs tiled across four different endogenous genomic loci of potential clinical interest in human HEK293T cells as determined by targeted amplicon sequencing of each on-target site using NGS (n=3, independent replicates). Note that these are the same crRNAs as used in FIG. 2H above. (C) Dot and bar plots showing indel frequencies induced by CasPhi2-17AA or WT CasPhi2 tested with crRNAs that target the BCL11A enhancer locus in HEK293T cells, as determined by targeted amplicon sequencing using NGS (left side; same data as shown in (B)). Right side shows the sequences and frequencies of indel alleles induced by CasPhi2-17AA and crRNA BCL11A-12 relative to the critically important GATA1 binding site known to be required for BCL11A enhancer activity and disruption of which has been shown in preclinical and Phase-I and II studies to enable re-induction of the expression of fetal hemoglobin (HbF) when edited with SpCas9 in human CD34+ cells. The spacer sequence of the BCL11A-12 crRNA is shown at the bottom of the right side of the figure. (D) Dot and bar plots showing indel frequencies (y-axes) induced by CasPhi2-17AA, WT CasPhi2, or a negative control when tested with five crRNAs targeting various endogenous gene loci in K562 and U2OS cells, as determined by targeted amplicon sequencing of each on-target site using NGS (n=2 or 3, independent replicates).

FIGS. 7A-7B. Testing the efficiencies of homology-directed repair (HDR) gene editing events mediated by the CasPhi2-17AA in human cells (A) Allele frequency table (derived from targeted amplicon NGS data) showing representative example of HDR-based ATG insertion edits induced with a crRNA targeting matched site 8 in HEK293T cells and an ssODN donor template (n=3). (B) Pie charts showing relative frequencies of wild-type (REF) alleles, alleles with indels (NHEJ), and alleles with precise HDR-mediated ATG insertion edits (HDR) induced with CasPhi2-17AA variant and a crRNA targeting VEGFA site 3, with and without an ssODN donor template, in HEK293T cells, as determined by targeted amplicon sequencing using NGS (n=3). A no treatment negative control is also shown for comparison.

FIGS. 8A-8D. Characterization of dCasPhi2-17AA variant-based Adenine Base Editors (Phi-ABEs) (A) Bar plots showing A-to-G base editing frequencies (y-axes) induced by various Phi-ABE fusion proteins. We tested “N-terminal TadA8e fusions” in which we fused the TadA8e adenosine deaminase to the N-terminal ends of CasPhi2-17AA, a “dead” CasPhi2-17AA variant with an additional E606Q mutation that impairs its catalytic nuclease activity, or another “dead” CasPhi2-17AA variant with an additional D394A mutation that inactivates its catalytic nuclease activity (labeled in the figure as “TadA8e-Casphi(17aa)”, “TadA8e-deadCasPhi(E606Q)”, or “TadA8e-deadCasPhi(D394A)”, respectively). We also tested “C-terminal TadA8e fusions” in which we fused the TadA8e adenosine deaminase to the C-terminal ends of CasPhi2-17AA, a “dead” CasPhi2-17AA variant with an additional E606Q mutation that impairs its catalytic nuclease activity, or another “dead” CasPhi2-17AA variant with an additional D394A mutation that inactivates its catalytic nuclease activity (labeled in the figure as “Casphi(17aa)-TadA8e”, “deadCasPhi(E606Q)-TadA8e”, or “deadCasPhi(D394A)-TadA8e”, respectively). We additionally tested (as negative controls) CasPhi2-17AA variant (labeled as “CasPhi-17AA” in the figure) and a no treatment control. Each fusion protein and negative control was tested with three crRNAs targeting different endogenous loci (ABE site 7, ABE site 10, and VEGFA site 3) in HEK293T cells, as determined by targeted amplicon sequencing of each on-target site using NGS (n=3 independent replicates). (B) Dot and bar plots showing A-to-G base editing frequencies (y-axes) induced by various fusions of TadA8e to the N-terminus of dCasPhi2-17AA (with a D394A mutation; hereafter referred to as “dCasPhi2-17AA (D394A)”) with intervening linkers of various lengths (32, 65, and 97 AA in length-see Table 5 below). We also tested untethered TadA8e deaminase with dCasPhi-17AA (D394A) and inlaid fusions of TadA8e deaminase within dCasPhi-17AA-(D394A) inserted at AA positions F653 or G362 within the CasPhi2 sequence. We also performed a no treatment control. Each of these configurations were tested with three crRNAs targeting different endogenous gene loci (ABE site 7, ABE site 10, and VEGFA site 3) in HEK293T cells, as determined by targeted amplicon sequencing of each on-target site using NGS (n=3 independent replicates). (C) Heat maps showing A-to-G adenine base editing frequencies across all adenines of the on-target spacers of various endogenous human gene loci (targeted with a crRNA) using Phi-ABE fusions comprising TadA8e adenosine deaminase fused to the N-terminus of dCasPhi2-17AA (D394A) with an intervening 32 AA linker. Data shown from experiments in which this Phi-ABE fusion was tested with13 crRNAs targeting endogenous human gene loci in HEK293T cells. Frequencies of edits were determined by targeted amplicon sequencing of each on-target site using NGS (n=3 independent replicates). (D) Violin plots showing relative A-to-G base editing efficiencies per base across all potential adenine positions in the protospacer, based on pooled NGS data from multiple sites tested with TadA8e-dCasPhi2-17AA (D394A) including data shown in (C).

FIGS. 9A-9B. Engineering dCasPhi2-17AA (D394A)-based gene activators for targeted epigenetic editing in human cells (A) Dot and bar plots showing fold-activation (y-axes) of the (D) 69 or IL2RA gene promoters in HEK293T cells targeted using pools of four crRNAs or five crRNAs, respectively, and either dWTCasPhi2 (D394A) or dCasPhi2-17AA (D394A) with a VPR transcriptional activation domain fused at their C-termini (shown as dWTCasPhi2 (D394A)-VPR or dCasPhi2-17AA (D394A)-VPR in the figure) (n=3 independent replicates). Fold-activation values were determined by calculating the level of mRNA expression of the target gene as measured by quantitative RT-PCR in the presence of the targeted crRNA(s) over that in the presence of a non-targeting crRNA (NT). (B) Dot and bar plots showing fold-activation (y-axes) of the CD69 or IL2RA gene promoters in HEK293T cells with individual and pooled crRNAs (four for CD69 and five for IL2RA) tested with dCasPhi2-17AA D394A)-VPR(n=3 independent replicates). Fold-activation values were calculated as in (A).

FIG. 10. Alignment of the amino acid sequences of ten CasPhi proteins, including CasPhi2 at the bottom. CasPhi2 variants with proven improvement in gene editing efficiencies are highlighted with an asterisk underneath the CasPhi2 amino acid sequence. The consensus sequence is shown on top.

FIG. 11A-11B. Systematic assessment of the impact of 82 different individual amino acid substitutions added to the CasPhi2-T355R mutant on gene editing activity in human cells. Bar plots show the mean fold-change of indel frequencies relative to CasPhi2-T355R (y-axis) observed with crRNAs targeting six different endogenous gene sites in HEK293T cells (n=1). Indel frequencies were determined by targeted amplicon sequencing and ‘no treatment’ was used as a negative control.

FIG. 12. Testing the importance of a-helix 7 mutations for CasPhi2 gene editing activity by comparing gene editing activities of CasPhi2-17AA (including six mutations within α-helix 7) and the new variants, CasPhi2-11AA (lacking any mutations in a-helix 7) and CasPhi2-11 (+1) AA (same mutations as CasPhi2-11A but with an additional L149R mutation in a-helix 7) at 16 different endogenous genomic loci (CD69 site 1, CD69 site 14, CD69 site 2, 1IL2RA site 1, IL2RA site 5, IL2RA site 23, IL2RA site 29, B2M site 10, PDCD1 site 11, BCL11A site 16, TRAC site 19, matched site 5.5, matched site 8.4, EMX1 site 1, FANCF site 1.6, VEGFA site 3.3) in HEK293T cells (n=1). Gene editing indel frequencies at target sites were determined by targeted amplicon sequencing and ‘no treatment’ was used as a negative control.

FIG. 13. Screening CasPhi2-11AA derivatives bearing additional amino acid substitutions for their gene editing abilities in human HEK293T cells. Bar plots show the mean fold-change of indel frequencies relative to CasPhi2-11AA (y-axis) observed with crRNAs targeting eight different endogenous gene sites (B2M site 2, FANCF site 1.6, PDCD1 site 6, matched site 5.2, VEGFA site 3, BCL11A site 9, matched site 5.3, EMX1 site 1) in HEK293T cells (n=1). Indel frequencies were determined by targeted amplicon sequencing and ‘no treatment’ was used as a negative control. 36 variants with comparable or higher activity than CasPhi2-11AA are indicated with an asterisk (*).

FIG. 14A-14B. Gene editing activities of 20 combinatorial variants of CasPhi2 at 8 endogenous genomic loci in HEK293T cells (n=2, independent replicates). Indel frequencies were determined by targeted amplicon sequencing and ‘no treatment’ was used as a negative control. (A) Bar graph showing mean indel frequencies (y-axis) induced by the 20 variants and the CasPhi2-DM, CasPhi2-11AA, and CasPhi2-17AA variants with the ABE site 5, B2M site 10, TRAC site 10, EMX1 site 1, FANCF site 1.1, matched site 5.5, matched site 8.1 and PDCD1 site3 crRNAs. Two highly active variants (#1 and #2) are marked with an asterisk (*). (B) Bar graph showing mean indel frequencies (y-axis) induced by variants #1 and #2 (labeled here as CasPhi2-15AAx7 and CasPhi2-14AAx7, respectively), CasPhi2-11AA, and CasPhi2-17AA at each of the eight endogenous gene sites tested.

### DETAILED DESCRIPTION

Despite the discovery and initial optimization of various smaller-size Cas nucleases, there remains no hypercompact nuclease that functions robustly and efficiently in human cells both as a nuclease and when fused to other functional domains (e.g., for use as a base editor or epigenetic editor).

Specifically, while other Cas proteins with reduced size have been described, these enzymes potentially require dimerization to function efficiently (Cas12f)¹² which could complicate their therapeutic use when compared to monomeric Cas proteins, such as CasPhi2 (Cas12j-2). Another potential disadvantage of Cas12f systems might be their relatively extensive and longer length crRNAs, which lead to Cas12f ribonucleoproteins (RNPs) having a higher molecular weight than CasPhi2¹⁵. Furthermore, AsCas12f, the smallest Cas12f protein (422aa) with the most useful PAM requirement (5′NTTR) shows the lowest editing efficiencies of a range of miniature Cas12f systems in human cells¹⁷. This might be explained in part by its biochemical properties: it is a thermophilic nuclease with severely reduced activity at 37° C.⁹.

Here we describe the testing of the phage-derived CasPhi2 nuclease on a large series of endogenous gene targets and report the surprising finding that, contrary to previous published studies, its editing efficiency is surprisingly inefficient in human cells.

Using multiple rounds of protein engineering, we constructed multiple CasPhi2 variants that have up to 13,000-fold increases in their gene editing activities in human cells relative to the original wild-type enzyme. We used one of these highly active variants to create base editors and epigenetic editors that function efficiently in human cells.

### Engineered CasPhi2 Variants

Provided herein are CasPhi2 variants. The CasPhi2 wild type sequence is as follows (GenBank Accession No. 7LYS_A; Pausch P, Soczek K M, Herbst D A, Tsuchida C A, Al-Shayeb B, Banfield J F, Nogales E, Doudna J A. DNA interference states of the hypercompact CRISPR-CasΦ effector. Nat Struct Mol Biol. 2021 Aug.; 28 (8): 652-661):

```
(SEQ ID NO: 1)
    1 MPKPAVESEF SKVLKKHFPG ERFRSSYMKR
        GGKILAAQGE EAVVAYLQGK SEEEPPNFQP
  61 PAKCHVVTKS RDFAEWPIMK ASEAIQRYIY
        ALSTTERAAC KPGKSSESHA AWFAATGVSN
121 HGYSHVQGLN LIFDHTLGRY DGVLKKVQLR
        NEKARARLES INASRADEGL PEIKAEEEEV
181 ATNETGHLLQ PPGINPSFYV YQTISPQAYR
        PRDEIVLPPE YAGYVRDPNA PIPLGVVRNR
241 CDIQKGCPGY IPEWQREAGT AISPKTGKAV
        TVPGLSPKKN KRMRRYWRSE KEKAQDALLV
301 TVRIGTDWVV IDVRGLLRNA RWRTIAPKDI
        SLNALLDLFT GDPVIDVRRN IVTFTYTLDA
361 CGTYARKWTL KGKQTKATLD KLTATQTVAL
        VAIDLGQTNP ISAGISRVTQ ENGALQCEPL
421 DRFTLPDDLL KDISAYRIAW DRNEEELRAR
        SVEALPEAQQ AEVRALDGVS KETARTQLCA
481 DFGLDPKRLP WDKMSSNTTF ISEALLSNSV
        SRDQVFFTPA PKKGAKKKAP VEVMRKDRTW
541 ARAYKPRLSV EAQKLKNEAL WALKRTSPEY
        LKLSRRKEEL CRRSINYVIE KTRRRTQCQI
601 VIPVIEDLNV RFFHGSGKRL PGWDNFFTAK
        KENRWFIQGL HKAFSDLRTH RSFYVFEVRP
661 ERTSITCPKC GHCEVGNRDG EAFQCLSCGK
        TCNADLDVAT HNLTQVALTG KTMPKREEPR
721 DAQGTAPARK TKKASKSKAP PAEREDQTPA
        QEPSQTS
```

The CasPhi2 variants described herein can include mutations at one or more of the following positions: T355 and/or D679 (or at positions analogous thereto). In some embodiments, the CasPhi2 variants described herein can include a mutation at T355. In some embodiments, the CasPhi2 variants described herein can include a mutation at D679. In some embodiments, the CasPhi2 variants described herein can include mutations at T355 and D679. In some embodiments, the mutation at T335 is T355R or T355K. In some embodiments, the mutation at D679 is D679R, D679K, D679H, or D679T.

In some embodiments, the CasPhi2 variants include mutations at one or both of positions T355 and D679, and one or more mutations at one of the following positions: S11, S25, A36, S106, E107, S124, D134, G138, L149, A156, E159, S160, S164, D167, E168, T203, P233, D337, A261, P277, T357, L370, D427, D428, A435, N₄₉₇, L506, S507, N508, S509, S511, D513, Q514, T518, P519, A520, P521, G524, A525, K526, K527, P530, V531, E532, V533, R538, T539, A543 E569, L571, S574, E578, S616, T628, T649, E674, Q684, and/or T691.

In some embodiments, the CasPhi2 variants include a mutation at position T355 and one or more mutations at one of the following positions: S11, S25, A36, S106, D134, L149, A156, E159, S160, S164, D167, E168, T203, A261, P277, D337, T357, L370, D427, D428,,, A435, N497, L506, S507, N508, S509, S511, D513, Q514, T518, P519, A520, G524, A525, K526, K527, P530, V531, E532, V533, R538, T539, A543, E569, L571, E578, S616, T628, T649, E674, G676, D679, Q684, and/or T691.

In some embodiments, the CasPhi2 variants include one of the sets of mutations shown in Table 1 below:

TABLE 1
Combinatorial CasPhi2 variants.

| No. | CasPhi2 Variant |
|---|---|
| 1 | A36R/L149R/T355R/D679K |
| 2 | A36R/D167K/T355R/D679K |
| 3 | A36R/L571K/T355R/D679K |
| 4 | A36R/S616R/T355R/D679K |
| 5 | L149R/D167K/T355R/D679K |
| 6 | L149R/L571K/T355R/D679K |
| 7 | L149R/S616R/T355R/D679K |
| 8 | D167K/S616R/T355R/D679K |
| 9 | D167K/L571K/T355R/D679K |
| 10 | L571K/S616R/T355R/D679K |
| 11 | S106R/D134R/T355R/D679K |
| 12 | S106R/S164K/T355R/D679K |
| 13 | S106R/E168K/T355R/D679K |
| 14 | S106R/P277R/T355R/D679K |
| 15 | S106R/T357K/T355R/D679K |
| 16 | S106R/T518R/T355R/D679K |
| 17 | S106R/E578K/T355R/D679K |
| 18 | S106R/T649R/T355R/D679K |
| 19 | S106R/Q684R/T355R/D679K |
| 20 | S106R/T691R/T355R/D679K |
| 21 | D134R/S164K/T355R/D679K |
| 22 | D134R/E168K/T355R/D679K |
| 23 | D134R/P277R/T355R/D679K |
| 24 | D134R/T357K/T355R/D679K |
| 25 | D134R/T518R/T355R/D679K |
| 26 | D134R/E578K/T355R/D679K |
| 27 | D134R/T649R/T355R/D679K |
| 28 | D134R/Q684R/T355R/D679K |
| 29 | D134R/T691R/T355R/D679K |
| 30 | S164K/P277R/T355R/D679K |
| 31 | S164K/T357K/T355R/D679K |
| 32 | S164K/T518R/T355R/D679K |
| 33 | S164K/E578K/T355R/D679K |
| 34 | S164K/T649R/T355R/D679K |
| 35 | S164K/Q684R/T355R/D679K |
| 36 | S164K/T691R/T355R/D679K |
| 37 | E168K/P277R/T355R/D679K |
| 38 | E168K/T357K/T355R/D679K |
| 39 | E168K/T518R/T355R/D679K |
| 40 | E168K/E578K/T355R/D679K |
| 41 | E168K/T649R/T355R/D679K |
| 42 | E168K/Q684R/T355R/D679K |
| 43 | E168K/T691R/T355R/D679K |
| 44 | P277R/T357K/T355R/D679K |
| 45 | P277R/T518R/T355R/D679K |
| 46 | P277R/E578K/T355R/D679K |
| 47 | P277R/T649R/T355R/D679K |
| 48 | P277R/Q684R/T355R/D679K |
| 49 | P277R/T691R/T355R/D679K |
| 50 | T357K/T518R/T355R/D679K |
| 51 | T357K/E578K/T355R/D679K |
| 52 | T357K/T649R/T355R/D679K |
| 53 | T357K/Q684R/T355R/D679K |
| 54 | T357K/T691R/T355R/D679K |
| 55 | T518R/E578K/T355R/D679K |
| 56 | T518R/T649R/T355R/D679K |
| 57 | T518R/Q684R/T355R/D679K |
| 58 | T518R/T691R/T355R/D679K |
| 59 | E578K/T649R/T355R/D679K |
| 60 | E578K/Q684R/T355R/D679K |
| 61 | E578K/T691R/T355R/D679K |
| 62 | T649R/Q684R/T355R/D679K |
| 63 | T649R/T691R/T355R/D679K |
| 64 | Q684R/T691R/T355R/D679K |
| 65 | A36R/L149R/D167K/T355R/D679K |
| 66 | A36R/L149R/L571K/T355R/D679K |
| 67 | A36R/L149R/S616R/T355R/D679K |
| 68 | A36R/D167K/L571K/T355R/D679K |
| 69 | A36R/D167K/S616R/T355R/D679K |
| 70 | A36R/L571K/S616R/T355R/D679K |
| 71 | L149R/D167K/L571K/T355R/D679K |
| 72 | L149R/D167K/S616R/T355R/D679K |
| 73 | L149R/L571K/S616R/T355R/D679K |
| 74 | D167K/L571K/S616R/T355R/D679K |
| 75 | A36R/L149R/D167K/L571K/S616R/T355R/D679K |
| 76 | A36R/L149R/D167K/L571K/T355R/D679K |
| 77 | A36R/D167K/L571K/S616R/T355R/D679K |
| 78 | A36R/L149R/L571K/S616R/T355R/D679K |
| 79 | A36R/L149R/D167K/S616R/T355R/D679K |
| 80 | L149R/D167K/L571K/S616R/T355R/D679K |
| 81 | S164K/D167K/E168K/T355R/D679K |
| 82 | S164K/D167K/T355R/D679K |
| 83 | S164K/E168K/T355R/D679K |
| 84 | D167K/E168K/T355R/D679K |
| 85 | A36R/L149R/S164K/E168K/L571K/S616R/T355R/D679K |
| 86 | A36R/L149R/S164K/D167K/E168K/L571K/S616R/T355R/D679K |
| 87 | A36R/L149R/E168K/L571K/S616R /T355R/D679K |
| 88 | A36R/L149R/D167K/E168K/L571K/S616R/T355R/D679K |
| 89 | E159R/S160K/S164K/D167K/E168K/T355R/D679K |
| 90 | E159R/S160K/T355R/D679K |
| 91 | A36R/S106R/L149R/D167K/L571K/S616R/T355R/D679K |
| 92 | A36R/D134R/L149R/D167K/L571K/S616R/T355R/D679K |
| 93 | A36R/L149R/D167K/P277R/L571K/S616R/T355R/D679K |
| 94 | A36R/L149R/D167K/T357K/L571K/S616R/T355R/D679K |
| 95 | A36R/L149R/D167K/T518R/L571K/S616R/T355R/D679K |
| 96 | A36R/L149R/D167K/L571K/E578K/S616R/T355R/D679K |
| 97 | A36R/L149R/D167K/L571K/S616R/Q684R/T355R/D679K |
| 98 | A36R/L149R/D167K/L571K/S616R/T691R/T355R/D679K |
| 99 | S106R/L149R/D167K/L571K/T355R/D679K |
| 100 | D134R/L149R/D167K/L571K/T355R/D679K |
| 101 | L149R/D167K/P277R/L571K/T355R/D679K |
| 102 | L149R/D167K/T357K/L571K/T355R/D679K |
| 103 | L149R/D167K/T518R/L571K/T355R/D679K |
| 104 | L149R/D167K/L571K/E578K/T355R/D679K |
| 105 | L149R/D167K/L571K/Q684R/T355R/D679K |
| 106 | L149R/D167K/L571K/T691R/T355R/D679K |
| 107 | A36R/D134R/L149R/D167K/T357K/L571K/S616R/T355R/D679K |
| 108 | A36R/L149R/D167K/P277R/T357K/L571K/S616R/T355R/D679K |
| 109 | A36R/L149R/D167K/P277R/L571K/E578K/S616R/T355R/D679K |
| 110 | A36R/L149R/D167K/T357K/L571K/E578K/S616R/T355R/D679K |
| 111 | D134R/L149R/D167K/T357K/L571K/T355R/D679K |
| 112 | L149R/D167K/P277R/T357K/L571K/T355R/D679K |
| 113 | L149R/D167K/P277R/L571K/E578K/T355R/D679K |
| 114 | L149R/D167K/T357K/L571K/E578K/T355R/D679K |
| 115 | A36R/D134R/L149R/D167K/P277R/T357K/L571K/S616R/T355R/D679K |
| 116 | A36R/L149R/D167K/P277R/T357K/L571K/S616R/Q684R/T355R/D679K |
| 117 | A36R/S106R/L149R/D167K/P277R/T357K/L571K/S616R/T355R/D679K |
| 118 | A36R/L149R/D167K/P277R/T357K/T518R/L571K/S616R/T355R/D679K |
| 119 | A36R/S106R/L149R/D167K/P277R/T357K/L571K/S616R/Q684R/T355R/D679K |
| 120 | A36R/S106R/L149R/D167K/P277R/T357K/T518R/L571K/S616R/T355R/D679K |
| 121 | A36R/L149R/D167K/P277R/T357K/T518R/L571K/S616R/Q684R/T355R/D679K |
| 122 | A36R/S106R/D134R/L149R/D167K/P277R/T357K/L571K/S616R/T355R/D679K |
| 123 | A36R/D134R/L149R/D167K/P277R/T357K/L571K/S616R/Q684R/T355R/D679K |
| 124 | A36R/D134R/L149R/D167K/P277R/T357K/T518R/L571K/S616R/T355R/D679K |
| 125 | A36R/S106R/D134R/L149R/D167K/P277R/T357K/T518R/L571K/S616R/T355R/D679K |
| 126 | A36R/S106R/D134R/L149R/D167K/P277R/T357K/L571K/S616R/Q684R/T355R/D679K |
| 127 | A36R/D134R/L149R/D167K/P277R/T357K/T518R/L571K/S616R/Q684R/T355R/D679K |
| 128 | A36R/S106R/L149R/D167K/P277R/T357K/T518R/L571K/S616R/Q684R/T355R/D679K |
| 129 | A36R/S106R/D134R/L149R/D167K/P277R/T357K/T518R/L571K/S616R/Q684R/T355R/D679K |
| 130 | A36R/S106R/D134R/L149R/D167K/P277R/T357K/T518R/L571K/S616R/Q684R/T691R/T355R/D679K |
| 131 | S106R/D134R/L149R/D167K/P277R/T357K/T518R/L571K/Q684R/T691R/T355R/D679K |
| 132 | A36R/L149R/E159A/S160A/S164A/D167K/E168A/P277R/T357K/L571K/S616R/T355R/D679K |
| 133 | A36R/S106R/D134R/L149R/E159A/S160A/S164A/D167K/E168A/P277R/T357K/T518R/L571K/S616R/Q684R/T355R/D679K |
| 134 | A36R/S106R/D134R/L149R/E159A/S160A/S164A/D167K/E168A/P277R/T357K/L571K/S616R/Q684R/T355R/D679K |
| 135 | A36R/D134R/L149R/E159A/S160A/S164A/D167K/E168A/P277R/T357K/L571K/S616R/Q684R/T355R/D679K |
| 136 | E159A/S160A/S164A/D167K/E168A/T355R/D679K |
| 137 | A36R/S106R/D134R/P277R/T355R/T357K/T518R/L571K/S616R/D679K/Q684R |
| 138 | A36R/S106R/D134R/L149R/P277R/T355R/T357K/T518R/L571K/S616R/D679K/Q684R |
| 139 | A36K, S106K, D134K, P277K, D337K, T355R, T357K, V531R, T539A, A543K, L571K, S616K, D679K, |
| (CasPhi2- | Q684R, T691K |
| 15AAx7) | |
| 140 | A36K, S106K, D134K, P277K, D337K, T355R, T357K, V531R, T539A, A543K, L571K, S616K, D679K, T691K |
| (CasPhi2- | |
| 14AAx7) | |
| 141 | A36K, S106K, D134K, P277K, D337K, T355R, T357K, A520R, V531R, T539A, A543K, L571K, S616K, D679K, |
| | Q684R, T691K |
| 142 | S11R, A36R, S106R, D134R, P277R, D337K, T355R, T357K, T518R, A543R, L571K, S616R, D679K, Q684R, T691K |
| 143 | D337K, T355R |
| 144 | D337K, T355R, D679K |
| 145 | D337K, T355R, L571K, D679K |
| 146 | D337K T355R, E578K, D679K |
| 147 | D337K, T355R, L571K, E578K, D679K |
| 148 | T355R, T357K, S509K, A520R, V531R, T539A, A543K, L571K, D679K |
| 149 | T355R, T357K, S509K, A520R, V531R, T539A, A543K, L571K, S616K, D679K, Q684R, T691K |
| 150 | A36K, S106K, D134K, P277K, D337K, T355R, D679K |
| 151 | A36K, S106K, D134K, P277K, D337K, T355R, T357K, D679K |
| 152 | A36K, S106K, D134K, P277K, T355R, T357K, D679K |
| 153 | A36K, S106K, D134K, P277K, D337K, T355R, A543K, L571K, D679K |
| 154 | A36K, S106K, D134K, P277K, D337K, T355R, T357K, A543K, L571K, S616K, D679K, Q684R, T691K |
| 155 | A36K, S106K, D134K, P277K, D337K, T355R, T357K, A543K, L571K, S616K, D679K, T691K |
| 156 | A36K, S106K, D134K, P277K, D337K, T355R, A543K, L571K, S616K, D679K, T691K |
| 157 | A36K, S106K, D134K, P277K, D337K, T355R, A543K, L571K, S616K, D679K, Q684R, T691K |
| 158 | S11R, A36R, S106R, D134R, P277R, D337K, T355R, T357K, T518R, A543R, T571K, S616R |

In some embodiments, the CasPhi2 variants include the following mutations: A36R, S106R, D134R, P277R, T355R, T357K, T518R, L571K, S616R, D679K, and Q684R. In some instances, the variants including mutations at A36R, S106R, D134R, P277R, T355R, T357K, T518R, L571K, S616R, D679K, and Q684R further include one or more mutations at the following positions: S11, F23, S25, S26, E107, S124, G138, P196, T203, D213, E214, D227, N229, P233, L234, G249, A261, E290, G305, T306, N333, D337, T340, D342, C361, D428, A435, A439, D467, N497, F500, A504, L506, S507, N508, S509, V510, S511, D513, Q514, V515, P519, A520, P521, K522, K523, G524, A525, K526, K527, K528, A529, P530, V531, E532, V533, R538, T539, R542, A543, V550, E569, S574, E578, E579, C581, E590, T628, T649, E674, T691, and/or R716.

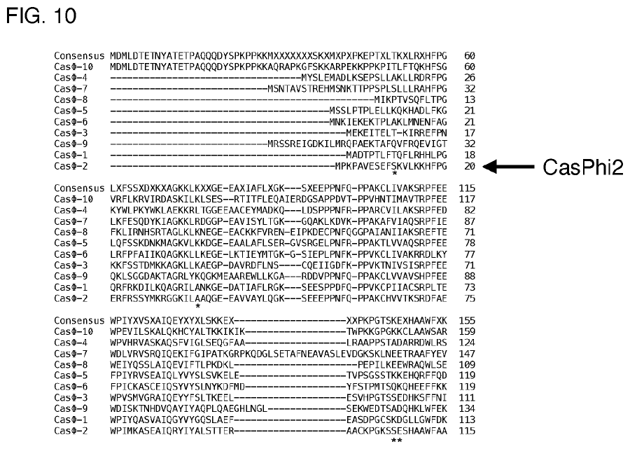

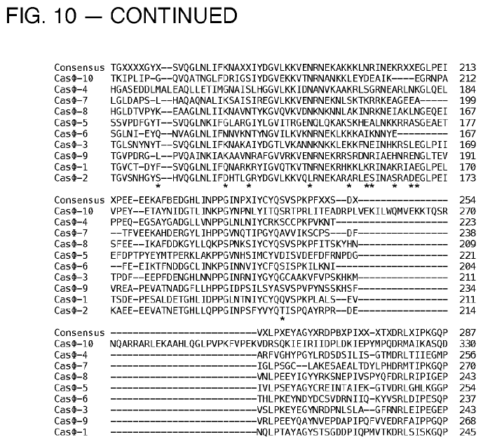

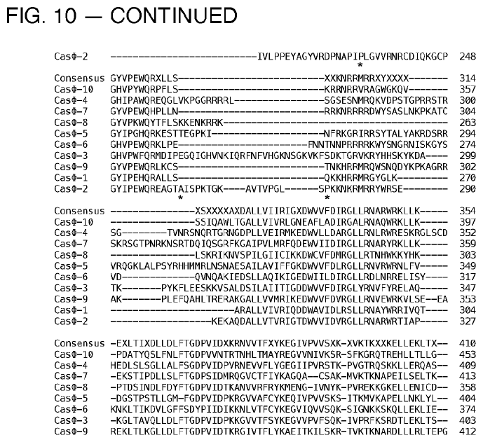

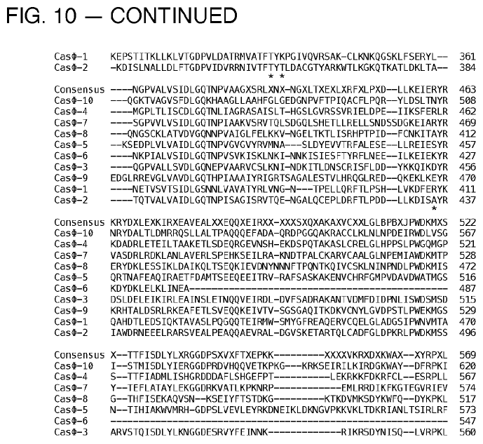

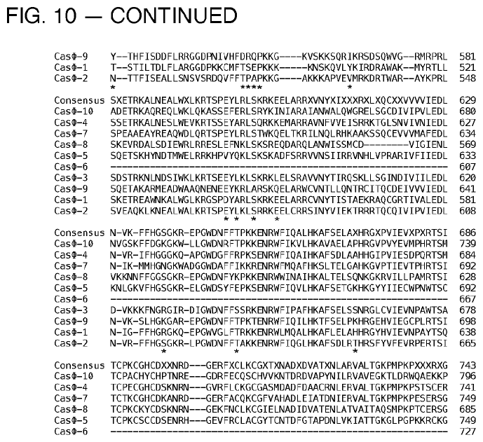

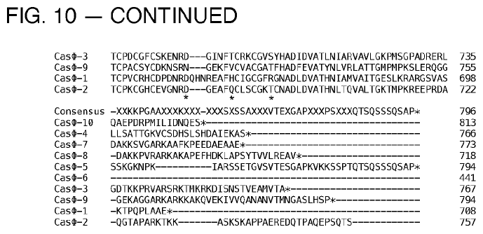

In some embodiments, the CasPhi2 variants are at least 70%, e.g., at least 75%, at least 80%, at least 85%, at least 90%, or at least 95% identical to the amino acid sequence of SEQ ID NO:1, e.g., have differences at up to 5%, 10%, 15%, 20%, 25%, or 30% of the amino acid residues of SEQ ID NO: 1 replaced, e.g., with conservative mutations, in addition to mutations described herein. In preferred embodiments, the variant retains or has improved desired activity of the parent, e.g., the nuclease activity (except where the parent is a nickase or a dead CasPhi2), and/or the ability to interact with a guide RNA and target DNA). See FIG. 10, which shows the alignment between various CasPhi proteins.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215:403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned using the BLAST algorithm and the default parameters.

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In some embodiments, the CasPhi2 variants also includes a mutation at D394, which inactivates the nuclease activity of the CasPhi2, to render the nuclease portion of the protein catalytically inactive; substitutions at these positions could be alanine (e.g., D394A), or other residues, e.g., glutamine, asparagine, tyrosine, serine, glycine, or glutamate. Variants carrying this mutation are referred to as dCasPhi2.

In some embodiments, the CasPhi2 variants also includes a mutation at E606, which impairs the nuclease activity of the CasPhi2, to render the nuclease portion of the protein catalytically impaired; substitutions at these positions could be glutamine (e.g., E606Q), or other residues, e.g., alanine, asparagine, tyrosine, serine, or aspartate. We also refer to this as a dCasPhi2 or dWT CasPhi2 variant.

### Fusions Including CasPhi2 Nucleases

In addition, the variants described herein can be used in fusion proteins in place of the wild-type CasPhi2 or other CasPhi2 mutants (such as the dCasPhi2) as known in the art, e.g., a fusion protein with a heterologous functional domains as described in U.S. Pat. No. 8,993,233; US20140186958; U.S. Pat. No. 9,023,649; WO/2014/099744; WO 2014/089290; WO2014/144592; WO144288; WO2014/204578; WO2014/152432; WO2115/099850; U.S. Pat. No. 8,697,359; US2010/0076057; US2011/0189776; US2011/0223638; US2013/0130248; WO/2008/108989; WO/2010/054108; WO/2012/164565; WO/2013/098244; WO/2013/176772; US20150050699; US20150071899 and WO 2014/124284.

For example, the CasPhi2 variants, can be fused to a heterologous functional domain on the N-terminus or C-terminus. In some embodiments, the CasPhi2 variant can have a heterologous functional domain that is inlaid within the nuclease (i.e., internally inserted). In some embodiments, the CasPhi2 variants also preferably comprise one or more nuclease-inactivating (e.g., mutation at D394) or nuclease-impairing mutation (e.g., mutation at E606).

In some embodiments, the heterologous functional domain is a transcriptional activation domain (e.g., a transcriptional activation domain from the VP16 domain from herpes simplex virus (Sadowski et al., 1988, Nature, 335:563-564) or VP64; the p65 domain from the cellular transcription factor NF-kappaB (Ruben et al., 1991, Science, 251:1490-93); or a tripartite effector fused to dCasPhi2, composed of activators VP64, p65, and Rta (VPR) linked in tandem, Chavez et al., Nat Methods. 2015 Apr.; 12 (4): 326-8) or other heterologous functional domains (e.g., transcriptional repressors (e.g., KRAB, ERD, SID, and others, e.g., amino acids 473-530 of the ets2 repressor factor (ERF) repressor domain (ERD), amino acids 1-97 of the KRAB domain of KOX1, or amino acids 1-36 of the Mad mSIN3 interaction domain (SID); see Beerli et al., PNAS USA 95:14628-14633 (1998)) or silencers such as Heterochromatin Protein 1 (HP1, also known as swi6), e.g., HP1α or HP1β; proteins or peptides that could recruit long non-coding RNAs (lncRNAs) fused to a fixed RNA binding sequence such as those bound by the MS2 coat protein, endoribonuclease Csy4, or the lambda N protein; base editors (enzymes that modify the methylation state of DNA (e.g., DNA methyltransferase (DNMT) or TET proteins); or enzymes that modify histone subunits (e.g., histone acetyltransferases (HAT), histone deacetylases (HDAC), histone methyltransferases (e.g., for methylation of lysine or arginine residues) or histone demethylases (e.g., for demethylation of lysine or arginine residues)) as are known in the art can also be used. A number of sequences for such domains are known in the art, e.g., a domain that catalyzes hydroxylation of methylated cytosines in DNA. Exemplary proteins include the Ten-Eleven-Translocation (TET) 1-3 family, enzymes that converts 5-methylcytosine (5-mC) to 5-hydroxymethylcytosine (5-hmC) in DNA.

TABLE 2
Sequences for human TET1-3 known in the art

GenBank Accession Nos.

| Gene | Amino Acid | Nucleic Acid |
|---|---|---|
| TET1 | NP_085128.2 | NM_030625.2 |
| TET2* | NP_001120680.1 (var 1) | NM_001127208.2 |
| | NP_060098.3 (var 2) | NM_017628.4 |
| TET3 | NP_659430.1 | NM_144993.1 |

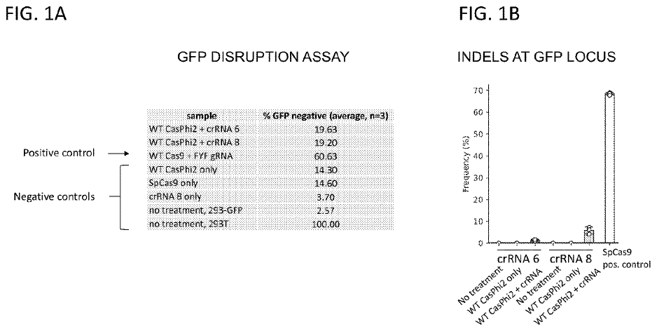

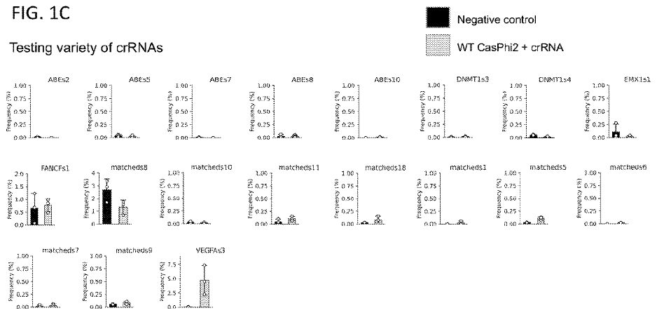

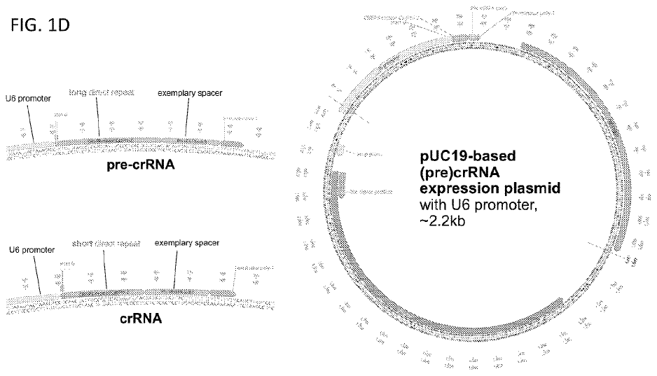

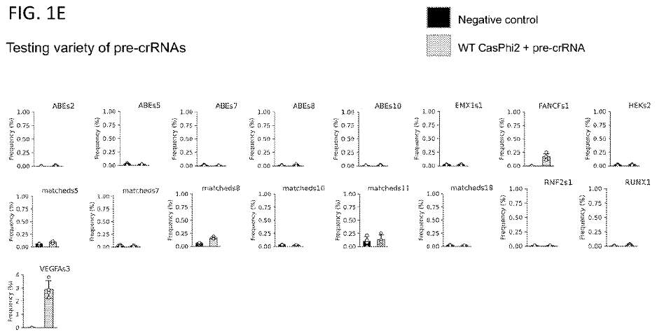

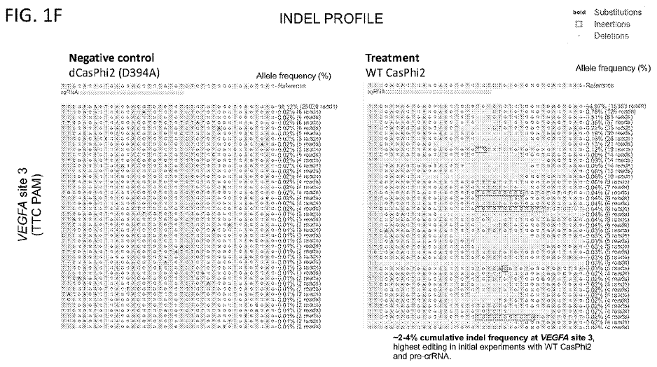

*Variant (var 1) represents the longer transcript and encodes the longer isoform (a).
Variant (var 2) differs in the 5′ UTR and in the 3′ UTR and coding sequence compared to variant 1.
The resulting isoform (b) is shorter and has a distinct C-terminus compared to isoform a. In some embodiments, all or part of the full-length sequence of the catalytic domain can be included, e.g., a catalytic module comprising the cysteine-rich extension and the 2OGFeDO domain encoded by 7 highly conserved exons, e.g., the Tet1 catalytic domain comprising amino acids 1580-2052, Tet2 comprising amino acids 1290-1905 and Tet3 comprising amino acids 966-1678. See, e.g., FIG. 1 of Iyer et al., Cell Cycle. 2009 Jun. 1; 8(11): 1698-710. Epub 2009 Jun. 27, for an alignment illustrating the key catalytic residues in all three Tet proteins, and the supplementary materials thereof (available at ftp site ftp.ncbi.nih.gov/pub/aravind/DONS/supplementary_material_DONS.html) for full length sequences (see, e.g., seq 2c); in some embodiments, the sequence includes amino acids 1418-2136 of Tet1 or the corresponding region in Tet2/3.

Other catalytic modules can be from the proteins identified in Iyer et al., 2009.

In some embodiments, the heterologous functional domain is a base editor, e.g., a deaminase that modifies cytosine DNA bases, e.g., a cytidine deaminase from the apolipoprotein B mRNA-editing enzyme, catalytic polypeptide-like (APOBEC) family of deaminases, including APOBEC1, APOBEC2, APOBEC3A, APOBEC3B, APOBEC3C, APOBEC3D/E, APOBEC3F, APOBEC3G, APOBEC3H, APOBEC4 (see, e.g., Yang et al., J Genet Genomics. 2017 Sep. 20; 44 (9): 423-437); activation-induced cytidine deaminase (AID), e.g., activation induced cytidine deaminase (AICDA), cytosine deaminase 1 (CDA1), and CDA2, and cytosine deaminase acting on tRNA (CDAT). The following table provides exemplary sequences; other sequences can also be used.

TABLE 3
Exemplary Sequences of Base Editors

| | GenBank Accession Nos. | |
|---|---|---|
| Deaminase | Nucleic Acid | Amino Acid |
| hAID/AICDA | NM_020661.3 isoform 1 | NP_065712.1 variant 1 |
| | NM_020661.3 isoform 2 | NP_065712.1 variant 2 |
| APOBEC1 | NM_001644.4 isoform a | NP_001635.2 variant 1 |
| | NM_005889.3 isoform b | NP_005880.2 variant 3 |
| APOBEC2 | NM_006789.3 | NP_006780.1 |
| APOBEC3A | NM_145699.3 isoform a | NP_663745.1 variant 1 |
| | NM_001270406.1 isoform b | NP_001257335.1 variant 2 |
| APOBEC3B | NM_004900.4 isoform a | NP_004891.4 variant 1 |
| | NM_001270411.1 isoform b | NP_001257340.1 variant 2 |
| APOBEC3C | NM_014508.2 | NP_055323.2 |
| APOBEC3D/E | NM_152426.3 | NP_689639.2 |
| APOBEC3F | NM_145298.5 isoform a | NP_660341.2 variant 1 |
| | NM_001006666.1 isoform b | NP_001006667.1 variant 2 |
| APOBEC3G | NM_021822.3 (isoform a) | NP_068594.1 (variant 1) |
| APOBEC3H | NM_001166003.2 | NP_001159475.2 (variant SV-200) |
| APOBEC4 | NM_203454.2 | NP_982279.1 |
| CDA1* | NM_127515.4 | NP_179547.1 |
| pmCDA1** | PMID 27492474 | PMID 27492474 |

*from Saccharomyces cerevisiae S288C
*from sea lamprey (Petromyzon marinus)

In some embodiments, the heterologous functional domain is a deaminase that modifies adenosine DNA bases, e.g., the deaminase is an adenosine deaminase 1 (ADA1), ADA2; adenosine deaminase acting on RNA 1 (ADAR1), ADAR2, ADAR3 (see, e.g., Savva et al., Genome Biol. 2012 Dec. 28; 13 (12): 252); adenosine deaminase acting on tRNA 1 (ADAT1), ADAT2, ADAT3 (see Keegan et al., RNA. 2017 Sep.; 23 (9): 1317-1328 and Schaub and Keller, Biochimie. 2002 Aug.; 84 (8): 791-803); and naturally occurring or engineered tRNA-specific adenosine deaminase (TadA) (see, e.g., Gaudelli et al., Nature. 2017 Nov. 23; 551 (7681): 464-471) (NP_417054.2 (Escherichia coli str. K-12 substr. MG1655); See, e.g., Wolf et al., EMBO J. 2002 Jul. 15; 21 (14): 3841-51. The following table provides exemplary sequences; other sequences can also be used.

TABLE 4
Exemplary Sequences of Deaminases

| | GenBank Accession Nos. or PMID | |
|---|---|---|
| Deaminase | Nucleic Acid | Amino Acid |
| ADA (ADA1) | NM_000022.3 variant 1 | NP_000013.2 isoform 1 |
| ADA2 | NM_001282225.1 | NP_001269154.1 |
| ADAR | NM_001111.4 | NP_001102.2 |
| ADAR2 (ADARB1) | NM_001112.3 variant 1 | NP_001103.1 isoform 1 |
| ADAR3 (ADARB2) | NM_018702.3 | NP_061172.1 |
| ADAT1 | NM_012091.4 variant 1 | NP_036223.2 isoform 1 |
| ADAT2 | NM_182503.2 variant 1 | NP_872309.2 isoform 1 |
| ADAT3 | NM_138422.3 variant 1 | NP_612431.2 isoform 1 |
| TadA | LR883050.1: | CAD6006593.1 |
| | 1257244-1257747 | |
| TadA 7.10 | PMID 29160308 | PMID 29160308 |
| TadA 8e | PMID 32433547 | PMID 32433547 |
| TadA 8.17 | PMID 32284586 | PMID 32284586 |
| TadA 8.20 | PMID 32284586 | PMID 32284586 |
| TadA 8e-N108Q or | PMID 36229683 | PMID 36229683 |
| TadA8e-N108Q/ | | |
| L145T (ABE9) | | |

In some embodiments, the heterologous functional domain is an enzyme, domain, or peptide that inhibits or enhances endogenous DNA repair or base excision repair (BER) pathways, e.g., thymine DNA glycosylase (TDG; GenBank Acc Nos. NM_003211.4 (nucleic acid) and NP_003202.3 (protein)) or uracil DNA glycosylase (UDG, also known as uracil N-glycosylase, or UNG; GenBank Acc Nos. NM_003362.3 (nucleic acid) and NP_003353.1 (protein)) or uracil DNA glycosylase inhibitor (UGI) that inhibits UNG mediated excision of uracil to initiate BER (see, e.g., Mol et al., Cell 82, 701-708 (1995); Komor et al., Nature. 2016 May 19; 533(7603)); or DNA end-binding proteins such as Gam, which is a protein from the bacteriophage Mu that binds free DNA ends, inhibiting DNA repair enzymes and leading to more precise editing (less unintended base edits; Komor et al., Sci Adv. 2017 Aug. 30; 3(8):eaao4774).

In some embodiments, all or part of the protein, e.g., at least a catalytic domain that retains the intended function of the enzyme, can be used.

In some embodiments, the heterologous functional domain is a biological tether, and comprises all or part of (e.g., DNA binding domain from) the MS2 coat protein, endoribonuclease Csy4, or the lambda N protein. These proteins can be used to recruit RNA molecules containing a specific stem-loop structure to a locale specified by the dCasPhi2 variant gRNA targeting sequences. For example, a dCasPhi2 variant fused to MS2 coat protein, endoribonuclease Csy4, or lambda N can be used to recruit a long non-coding RNA (lncRNA) such as XIST or HOTAIR; see, e.g., Keryer-Bibens et al., Biol. Cell 100:125-138 (2008), that is linked to the Csy4, MS2 or lambda N binding sequence. Alternatively, the Csy4, MS2 or lambda N protein binding sequence can be linked to another protein, e.g., as described in Keryer-Bibens et al., supra, and the protein can be targeted to the dCasPhi2 variant binding site using the methods and compositions described herein. In some embodiments, the Csy4 is catalytically inactive. In some embodiments, the CasPhi2 variant, preferably a dCasPhi2 variant, is fused to FokI as described in U.S. Pat. No. 8,993,233; US20140186958; U.S. Pat. No. 9,023,649; WO/2014/099744; WO 2014/089290; WO2014/144592; WO144288; WO2014/204578; WO2014/152432; WO2115/099850; U.S. Pat. No. 8,697,359; US2010/0076057; US2011/0189776; US2011/0223638; US2013/0130248; WO/2008/108989; WO/2010/054108; WO/2012/164565; WO/2013/098244; WO/2013/176772; US20150050699; US20150071899 and WO 2014/204578.

In some embodiments, the fusion proteins include a linker between the CasPhi2 variant and the heterologous functional domains. Linkers that can be used in these fusion proteins (or between fusion proteins in a concatenated structure) can include any sequence that does not interfere with the function of the fusion proteins. In preferred embodiments, the linkers are short, e.g., 2-40 amino acids, and are typically flexible (i.e., comprising amino acids with a high degree of freedom such as glycine, alanine, and serine). In some embodiments, the linker comprises one or more units consisting of GGGS (SEQ ID NO:2) or GGGGS (SEQ ID NO:3), e.g., two, three, four, or more repeats of the GGGS (SEQ ID NO:2) or GGGGS (SEQ ID NO:3) unit. In some embodiments, the linker comprises an XTEN linker (e.g., a 32 amino acid modified XTEN linker (flanked with extended GlySer linkers on both sides)). Other linker sequences can also be used (see Table 5). 10

TABLE 5
Different linkers used to fuse dCasPhi2-17AA to deaminase domains

| Linker Name | AA sequence | SEQ ID NO: |
|---|---|---|
| Modified XTEN linker | SGGSSGGSSGSETPGTSESATPES | 4 |
| no. 1 = 32 aa linker | SGGSSGGS | |
| 33aa XTEN linker | SGGSSGGSSGSETPGTSESATPES | 5 |
| from PE + 32 aa linker | SGGSSGGSSSGGSSGGSSGSETPG | |
| from BE4max = 65 aa | TSESATPESSGGSSGGS | |
| linker | | |
| Modified 32 aa linker | SGGSSGGSSGSETPGTSESATPES | 6 |
| + 65 aa linker = 97 aa | SGGSSGGSSGGSSGGSSGSETPGT | |
| linker | SESATPESSGGSSGGSSSGGSSGG | |
| (32 aa + 33 aa + 32 aa | SSGSETPGTSESATPESSGGSSGG | |
| = 97 aa) | S | |
| GGGS linker | GGGS | 7 |
| GGGSGGGS linker | GGGSGGGS | 8 |
| PAP linker | PAP | 9 |
| PAPAP linker | PAPAP | 10 |
| PAPAPAP linker | PAPAPAP | 11 |
| 16aa XTEN linker | SGSETPGTSESATPES | 12 |
| from 32aa XTEN | | |
| linker | | |

In some embodiments, the variant protein includes a cell-penetrating peptide sequence that facilitates delivery to the intracellular space, e.g., HIV-derived TAT peptide, penetratins, transportans, or hCT derived cell-penetrating peptides, see, e.g., Caron et al., (2001) Mol Ther. 3 (3): 310-8; Langel, Cell-Penetrating Peptides: Processes and Applications (CRC Press, Boca Raton FL 2002); El-Andaloussi et al., (2005) Curr Pharm Des. 11 (28): 3597-611; and Deshayes et al., (2005) Cell Mol Life Sci. 62 (16): 1839-49.

Cell penetrating peptides (CPPs) are short peptides that facilitate the movement of a wide range of biomolecules across the cell membrane into the cytoplasm or other organelles, e.g. the mitochondria and the nucleus. Examples of molecules that can be delivered by CPPs include therapeutic drugs, plasmid DNA, oligonucleotides, siRNA, peptide-nucleic acid (PNA), proteins, peptides, nanoparticles, and liposomes. CPPs are generally 30 amino acids or less, are derived from naturally or non-naturally occurring protein or chimeric sequences, and contain either a high relative abundance of positively charged amino acids, e.g. lysine or arginine, or an alternating pattern of polar and non-polar amino acids. CPPs that are commonly used in the art include Tat (Frankel et al., (1988) Cell. 55:1189-1193, Vives et al., (1997) J. Biol. Chem. 272:16010-16017), penetratin (Derossi et al., (1994) J. Biol. Chem. 269:10444-10450), polyarginine peptide sequences (Wender et al., (2000) Proc. Natl. Acad. Sci. USA 97:13003-13008, Futaki et al., (2001) J. Biol. Chem. 276:5836-5840), and transportan (Pooga et al., (1998) Nat. Biotechnol. 16:857-861).

CPPs can be linked with their cargo through covalent or non-covalent strategies. Methods for covalently joining a CPP and its cargo are known in the art, e.g. chemical cross-linking (Stetsenko et al., (2000) J. Org. Chem. 65:4900-4909, Gait et al. (2003) Cell. Mol. Life. Sci. 60:844-853) or cloning a fusion protein (Nagahara et al., (1998) Nat. Med. 4:1449-1453). Non-covalent coupling between the cargo and short amphipathic CPPs comprising polar and non-polar domains is established through electrostatic and hydrophobic interactions.

CPPs have been utilized in the art to deliver potentially therapeutic biomolecules into cells. Examples include cyclosporine linked to polyarginine for immunosuppression (Rothbard et al., (2000) Nature Medicine 6 (11): 1253-1257), siRNA against cyclin B1 linked to a CPP called MPG for inhibiting tumorigenesis (Crombez et al., (2007) Biochem Soc. Trans. 35:44-46), tumor suppressor p53 peptides linked to CPPs to reduce cancer cell growth (Takenobu et al., (2002) Mol. Cancer Ther. 1 (12): 1043-1049, Snyder et al., (2004) PLOS Biol. 2: E36), and dominant negative forms of Ras or phosphoinositol 3 kinase (PI3K) fused to Tat to treat asthma (Myou et al., (2003) J. Immunol. 171:4399-4405).

CPPs have been utilized in the art to transport contrast agents into cells for imaging and biosensing applications. For example, green fluorescent protein (GFP) attached to Tat has been used to label cancer cells (Shokolenko et al., (2005) DNA Repair 4 (4): 511-518). Tat conjugated to quantum dots have been used to successfully cross the blood-brain barrier for visualization of the rat brain (Santra et al., (2005) Chem. Commun. 3144-3146). CPPs have also been combined with magnetic resonance imaging techniques for cell imaging (Liu et al., (2006) Biochem. and Biophys. Res. Comm. 347 (1): 133-140). See also Ramsey and Flynn, Pharmacol Ther. 2015 Jul. 22. pii: S0163-7258 (15) 00141-2.

In some embodiments, alternatively or in addition, the variant proteins can include a nuclear localization sequence, e.g., SV40 large T antigen NLS (PKKKRRV (SEQ ID NO:13)) and nucleoplasmin NLS (KRPAATKKAGQAKKKK (SEQ ID NO: 14)). Other NLSs are known in the art; see, e.g., Cokol et al., EMBO Rep. 2000 Nov. 15; 1 (5): 411-415; Freitas and Cunha, Curr Genomics. 2009 Dec.; 10 (8): 550-557. In some embodiments, the variants include a moiety that has a high affinity for a ligand, for example GST, FLAG or hexahistidine sequences. Such affinity tags can facilitate the purification of recombinant variant proteins.

For methods in which the variant proteins are delivered to cells, the proteins can be produced using any method known in the art, e.g., by in vitro translation, or expression in a suitable host cell from nucleic acid encoding the variant protein; a number of methods are known in the art for producing proteins. For example, the proteins can be produced in and purified from yeast, E. coli, insect cell lines, plants, transgenic animals, or cultured mammalian cells; see, e.g., Palomares et al., “Production of Recombinant Proteins: Challenges and Solutions,” Methods Mol Biol. 2004; 267:15-52. In addition, the variant proteins can be linked to a moiety that facilitates transfer into a cell, e.g., a lipid nanoparticle, optionally with a linker that is cleaved once the protein is inside the cell. See, e.g., LaFountaine et al., Int J Pharm. 2015 Aug. 13; 494(1): 180-194.

### Methods of Altering the Genome of a Cell

The variants described herein can be used for altering the genome of a cell; the methods generally include expressing the variant proteins in the cells, along with a guide RNA having a region complementary to a selected portion of the genome of the cell. Methods for selectively altering the genome of a cell are known in the art, see, e.g., U.S. Pat. No. 8,697,359; US2010/0076057; US2011/0189776; US2011/0223638; US2013/0130248; WO/2008/108989; WO/2010/054108; WO/2012/164565; WO/2013/098244; WO/2013/176772; US20150050699; US20150045546; US20150031134; US20150024500; US20140377868; US20140357530; US20140349400; US20140335620; US20140335063; US20140315985; US20140310830; US20140310828; US20140309487; US20140304853; US20140298547; US20140295556; US20140294773; US20140287938; US20140273234; US20140273232; US20140273231; US20140273230; US20140271987; US20140256046; US20140248702; US20140242702; US20140242700; US20140242699; US20140242664; US20140234972; US20140227787; US20140212869; US20140201857; US20140199767; US20140189896; US20140186958; US20140186919; US20140186843; US20140179770; US20140179006; US20140170753; Makarova et al., “Evolution and classification of the CRISPR-Cas systems” 9 (6) Nature Reviews Microbiology 467-477 (1-23) (June 2011); Wiedenheft et al., “RNA-guided genetic silencing systems in bacteria and archaea” 482 Nature 331-338 (Feb. 16, 2012); Gasiunas et al., “Cas9-crRNA ribonucleoprotein complex mediates specific DNA cleavage for adaptive immunity in bacteria” 109 (39) Proceedings of the National Academy of Sciences USA E2579-E2586 (Sep. 4, 2012); Jinek et al., “A Programmable Dual-RNA-Guided DNA Endonuclease in Adaptive Bacterial Immunity” 337 Science 816-821 (Aug. 17, 2012); Carroll, “A CRISPR Approach to Gene Targeting” 20 (9) Molecular Therapy 1658-1660 (September 2012); U.S. Appl. No. 61/652,086, filed May 25, 2012; Al-Attar et al., Clustered Regularly Interspaced Short Palindromic Repeats (CRISPRs): The Hallmark of an Ingenious Antiviral Defense Mechanism in Prokaryotes, Biol Chem. (2011) vol. 392, Issue 4, pp. 277-289; Hale et al., Essential Features and Rational Design of CRISPR RNAs That Function With the Cas RAMP Module Complex to Cleave RNAs, Molecular Cell, (2012) vol. 45, Issue 3, 292-302.

The variant proteins described herein can be used in place of the endonuclease proteins described in the foregoing references or in combination with analogous mutations described therein, with a guide RNA appropriate for the selected CasPhi2.

### Nucleic Acids

Also provided herein are isolated nucleic acids encoding the CasPhi2 variants, vectors comprising the isolated nucleic acids, optionally operably linked to one or more regulatory domains for expressing the variant proteins, and host cells, e.g., mammalian host cells, comprising the nucleic acids, and optionally expressing the variant proteins.

Guide RNAs (gRNAs)/CRISPR RNAs (crRNAs) for CasPhi2 and Variants

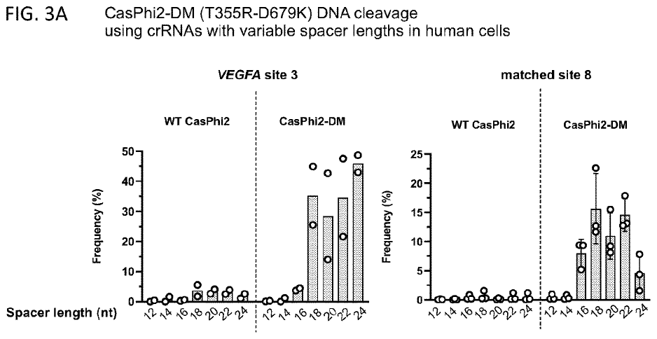

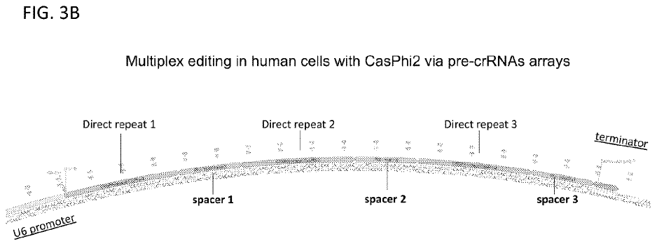

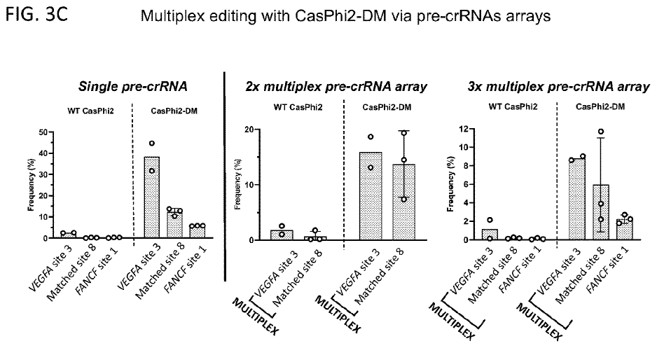

In contrast to Cas9 guide RNAs, which can consist of separate CRISPR RNAs (crRNAs) and tracrRNAs that function together to guide cleavage or chimeric fused crRNA-tracrRNAs (referred to as a single guide RNA or sgRNA, see also Jinek et al., Science 2012; 337:816-821), CasPhi nucleases (and CasPhi2 in particular) are guided to their target sites by a crRNA that contains a 5′ direct repeat and a 3′ spacer sequence (the latter being complementary to the target DNA sequence), without the need for a tracrRNA. These CasPhi crRNAs can be processed from arrays of pre-crRNAs (FIG. 3B) by the CasPhi nuclease itself, using the same RuvC domain that mediates DNA cleavage to cleave the crRNAs from these longer RNA transcripts¹⁶. In some embodiments, vectors (e.g., plasmids) encoding more than one CasPhi2 crRNA are used, e.g., plasmids encoding, 2, 3, 4, 5, or more crRNAs directed to different sites in the same region of the target gene.

CasPhi2 nucleases can be guided to specific genomic targets bearing a proximal protospacer adjacent motif (PAM) (e.g., 5′ TTN or 5′TBN PAMs, where B is G, T, or C), using a crRNA consisting of a 25 nt repeat (CAACGAUUGCCCCUCACGAGGGGAC; SEQ ID NO: 104) at its 5′ end and a 14-24 nt spacer sequence (also referred to herein as “spacer region,” “crRNA spacer,” or the like) at its 3′ end that is complementary to the “target strand” of the target DNA site (FIG. 1D). CasPhi2 nucleases can also be guided to genomic targets bearing a 5′ TTN or 5′ TBN PAM using a pre-crRNA consisting of a 36 nt repeat (GUCGGAACGCUCAACGAUUGCCCCUCACGAGGGGAC, SEQ ID NO: 105, at its 3′ end and a 14-24 nt spacer sequence at its 3′ end that is complementary to the “target strand” of the target DNA site (FIG. 1D and FIG. 3B).

In this application, we refer to the CasPhi2 crRNAs as “crRNAs”, “guide RNAs” or “gRNAs” and use these terms interchangeably.

In some embodiments, the crRNA or pre-crRNA harbors a 14 nt spacer sequence to enable nicking of the NTS, as had been shown in vitro for truncated crRNAs¹⁵. In some embodiments, the crRNA or pre-RNA harbors a 20 nt spacer sequence targeted clinically important endogenous human genes or their regulatory sequences (Table 6).

TABLE 6
Spacer sequences of CasPhi2 pre-crRNAs
or crRNAs targeted to clinically
important endogenous human genes
or their regulatory sequences
(sequences are shown 5′ to 3′)

| | | | SEQ |
|---|---|---|---|
| TARGET | | | ID |
| GENE | # | spacer sequence | No. |
| B2M | 1 | CUGAAGCUGACAGCAUUCGG | 32 |
| | 2 | CAGCAUUCGGGCCGAGAUGU | 33 |
| | 3 | GGGCCGAGAUGUCUCGCUCC | 34 |
| | 4 | CUCGCUCCGUGGCCUUAGCU | 35 |
| | 5 | CGCUCCGUGGCCUUAGCUGU | 36 |
| | 6 | GUGGCCUUAGCUGUGCUCGC | 37 |
| | 7 | CCUUAGCUGUGCUCGCGCUA | 38 |
| | 8 | GCUGUGCUCGCGCUACUCUC | 39 |
| | 9 | CGCUACUCUCUCUUUCUGGC | 40 |
| | 10 | CUGGCCUGGAGGCUAUCCAG | 41 |
| | 11 | UGGCCUGGAGGCUAUCCAGC | 42 |
| | 12 | CCUGGAGGCUAUCCAGCGUG | 43 |
| BCL11A | 1 | AAGCUAGUCUAGUGCAAGCU | 44 |
| | 2 | AAGCUAACAGUUGCUUUUAU | 45 |
| | 3 | CUUUUAUCACAGGCUCCAGG | 46 |
| | 4 | UAUCACAGGCUCCAGGAAGG | 47 |
| | 5 | AUCACAGGCUCCAGGAAGGG | 48 |
| | 6 | UCACAGGCUCCAGGAAGGGU | 49 |
| | 7 | AGGAAGGGUUUGGCCUCUGA | 50 |
| | 8 | GGCCUCUGAUUAGGGUGGGG | 51 |
| | 9 | GCCUCUGAUUAGGGUGGGGG | 52 |
| | 10 | UACCCCACCCACGCCCCCAC | 53 |
| | 11 | GAGGCCAAACCCUUCCUGGA | 54 |
| | 12 | CUGGAGCCUGUGAUAAAAGC | 55 |
| | 13 | AGCCUGUGAUAAAAGCAACU | 56 |
| | 14 | GAUAAAAGCAACUGUUAGCU | 57 |
| | 15 | UAAAAGCAACUGUUAGCUUG | 58 |
| | 16 | GCUUGCACUAGACUAGCUUC | 59 |
| | 17 | CACUAGACUAGCUUCAAAGU | 60 |
| | 18 | AAAGUUGUAUUGACCCUGGU | 61 |
| | 19 | AAGUUGUAUUGACCCUGGUG | 62 |
| | 20 | UAUUGACCCUGGUGUGUUAU | 63 |
| | 21 | AUUGACCCUGGUGUGUUAUG | 64 |
| | 22 | ACCCUGGUGUGUUAUGUCUA | 65 |
| | 23 | GACAUAACACACCAGGGUCA | 66 |
| | 24 | AUACAACUUUGAAGCUAGUC | 67 |
| PDCD1 | 1 | GGUGGGGCUGCUCCAGGCAU | 68 |
| | 2 | UCCAGGCAUGCAGAUCCCAC | 69 |
| | 3 | AGAUCCCACAGGCGCCCUGG | 70 |
| | 4 | CACAGGCGCCCUGGCCAGUC | 71 |
| | 5 | CCAGUCGUCUGGGCGGUGCU | 72 |
| | 6 | UCUGGGCGGUGCUACAACUG | 73 |
| | 7 | GGGCGGUGCUACAACUGGGC | 74 |
| | 8 | UACAACUGGGCUGGCGGCCA | 75 |
| | 9 | GCUGGCGGCCAGGAUGGUUC | 76 |
| | 10 | CCGCCAGCCCAGUUGUAGCA | 77 |
| | 11 | UAGCACCGCCCAGACGACUG | 78 |
| | 12 | CCAGGGCGCCUGUGGGAUCU | 79 |
| TRAC | 1 | UCCCACAGAUAUCCAGAACC | 80 |
| | 2 | CACAGAUAUCCAGAACCCUG | 81 |
| | 3 | AGAACCCUGACCCUGCCGUG | 82 |
| | 4 | CCCUGCCGUGUACCAGCUGA | 83 |
| | 5 | CGUGUACCAGCUGAGAGACU | 84 |
| | 6 | ACCAGCUGAGAGACUCUAAA | 85 |
| | 7 | GAGACUCUAAAUCCAGUGAC | 86 |
| | 8 | ACCGAUUUUGAUUCUCAAAC | 87 |
| | 9 | AUUCUCAAACAAAUGUGUCA | 88 |
| | 10 | UCAAACAAAUGUGUCACAAA | 89 |
| | 11 | UGAUGUGUAUAUCACAGACA | 90 |
| | 12 | UCUGUGAUAUACACAUCAGA | 91 |
| | 13 | CUUUGUGACACAUUUGUUUG | 92 |
| | 14 | UGACACAUUUGUUUGAGAAU | 93 |
| | 15 | UUUGAGAAUCAAAAUCGGUG | 94 |
| | 16 | AGAAUCAAAAUCGGUGAAUA | 95 |
| | 17 | UCACUGGAUUUAGAGUCUCU | 96 |
| | 18 | AGAGUCUCUCAGCUGGUACA | 97 |
| | 19 | GAGUCUCUCAGCUGGUACAC | 98 |
| | 20 | CUCAGCUGGUACACGGCAGG | 99 |
| | 21 | CAGCUGGUACACGGCAGGGU | 100 |
| | 22 | UACACGGCAGGGUCAGGGUU | 101 |
| | 23 | GGGUUCUGGAUAUCUGUGGG | 102 |
| | 24 | UGGAUAUCUGUGGGACAAGA | 103 |

The CasPhi2 gRNAs/crRNAs can include on the 5′ and/or 3′ ends additional X_{N} sequences, which can be any sequence (X is any nucleotide), wherein N (in the RNA) can be 1-200, e.g., 1-100, 1-50, or 1-20, that does not interfere with the binding of the ribonucleic acid to CasPhi2.

In some embodiments, the gRNA/crRNA includes one or more Adenine (A) or Uracil (U) nucleotides on the 3′ end. In some embodiments the RNA includes zero or more U, e.g., 0 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription of these RNAs from DNA expression vectors.

In some embodiments, the gRNA/crRNA is targeted to a site that is at least three or more mismatches different from any sequence in the rest of the genome in order to minimize off-target effects. In some embodiments, the guide RNA includes one or more Guanine (G) nucleotides at the 5′ end for enhanced expression from a U6 promoter from DNA expression vectors in mammalian cells. In some embodiments, the guide RNA includes one or more Guanine (G) nucleotides (e.g., one G or two G's at the 5′ end, preferably two Gs, i.e. 5′GG) at the 5′ end for enhanced expression from a T7 promoter for in vitro transcription (IVT) of the gRNA.

In some embodiments the one or more crRNA pre-crRNA comprises the following sequence:

SEQ ID NO: 106
5′-GCAACGAUUGCCCCUCACGAGGGGAC-N₁₂₋₂₄-U₀₋₈,
SEQ ID NO: 107
5′-GGUCGGAACGCUCAACGAUUGCCCCUCACGAGGGGA
C-N₁₂₋₂₄-U₀₋₈,
SEQ ID NO: 108
5′-GGCAACGAUUGCCCCUCACGAGGGGAC-N₁₂₋₂₄-U₀₋₈,
or
SEQ-ID No. 109
5′-GGGUCGGAACGCUCAACGAUUGCCCCUCACGAGGGG
AC-N₁₂₋₂₄-U₀₋₈.

Modified RNA oligonucleotides such as locked nucleic acids (LNAs) have been demonstrated to increase the specificity of RNA-DNA hybridization by locking the modified oligonucleotides in a more favorable (stable) conformation. For example, 2′-O-methyl RNA is a modified base where there is an additional covalent linkage between the 2′ oxygen and 4′ carbon which when incorporated into oligonucleotides can improve overall thermal stability and selectivity (Formula I).

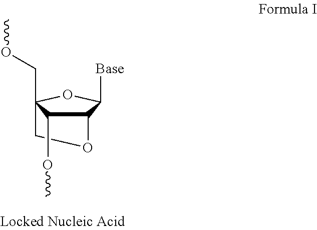

Thus in some embodiments, the gRNAs/crRNAs disclosed herein may comprise one or more modified RNA oligonucleotides. For example, the gRNA/crRNA molecules described herein can have one, some or all of the 17-18 or 17-19 nts 5′ region of the gRNA/crRNA spacer that is complementary to the target strand of the target sequence is/are modified, e.g., locked (2′-O-4′-C methylene bridge), 5′-methylcytidine, 2′-O-methyl-pseudouridine, or in which the ribose phosphate backbone has been replaced by a polyamide chain (peptide nucleic acid), e.g., a synthetic ribonucleic acid.

In other embodiments, one, some or all of the nucleotides of the gRNA/crRNA sequence may be modified, e.g., locked (2′-O-4′-C methylene bridge), 5′-methylcytidine, 2′-O-methyl-pseudouridine, or in which the ribose phosphate backbone has been replaced by a polyamide chain (peptide nucleic acid), e.g., a synthetic ribonucleic acid.

In some embodiments, the gRNAs and/or crRNAs can include one or more Adenine (A) or Uracil (U) nucleotides on the 3′ end.

Existing Cas9-based RNA-guided nucleases use gRNA-DNA heteroduplex formation to guide targeting to genomic sites of interest. However, RNA-DNA heteroduplexes can form a more promiscuous range of structures than their DNA-DNA counterparts. In effect, DNA-DNA duplexes are more sensitive to mismatches, suggesting that a DNA-guided nuclease may not bind as readily to off-target sequences, making them comparatively more specific than RNA-guided nucleases. Thus, the gRNA/crRNAs usable in the methods described herein can be hybrids, i.e., wherein one or more deoxyribonucleotides, e.g., a short DNA oligonucleotide, replaces all or part of the gRNA, e.g., all or part of the complementarity region of a gRNA. This DNA-based molecule could replace either all or part of the gRNA/crRNA. Such a system that incorporates DNA into the spacer complementarity region should more reliably target the intended genomic DNA sequences due to the general intolerance of DNA-DNA duplexes to mismatching compared to RNA-DNA duplexes. Methods for making such duplexes are known in the art, See, e.g., Barker et al., BMC Genomics. 2005 Apr. 22; 6:57; and Sugimoto et al., Biochemistry. 2000 Sep. 19; 39 (37): 11270-81.

In a cellular context, complexes of CasPhi2 with these synthetic gRNAs/crRNAs could be used to improve the genome-wide specificity of the CRISPR/Cas9 nuclease system.

The methods described can include expressing in a cell, or contacting the cell with, a CasPhi2 gRNA/crRNA plus a fusion protein as described herein.

### Expression Systems

To use the CasPhi2 variants described herein, it may be desirable to express them from a nucleic acid that encodes them. This can be performed in a variety of ways. For example, the nucleic acid encoding the CasPhi2 variant can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the CasPhi2 variant for production of the CasPhi2 variant. The nucleic acid encoding the CasPhi2 variant can also be cloned into an expression vector, for administration to a plant cell, animal cell, preferably a mammalian cell or a human cell, fungal cell, bacterial cell, or protozoan cell.

To obtain expression, a sequence encoding a CasPhi2 variant is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of fusion proteins. In contrast, when the CasPhi2 variant is to be administered in vivo for gene regulation, either a constitutive or an inducible promoter can be used, depending on the particular use of the CasPhi2 variant. In addition, a preferred promoter for administration of the CasPhi2 variant can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the CasPhi2 variant, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the CasPhi2 variant, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ.

For delivery of CasPhi2 and episomal expression of CasPhi2 and/or (pre) crRNAs in mammalian cells ex vivo or in vivo, adeno associated virus (AAV)-based vector systems or integration-deficient lentiviruses (IDLV) can be used. For ex vivo integration of CasPhi2 sequences in the cellular genome, lentiviruses or gammaretroviruses could be used as vector systems.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the CasPhi2 variants can include RNA Pol III promoters to drive expression of the crRNAs or pre-crRNAs, e.g., the H1, U6 or 7SK promoters. These promoters allow for expression of the crRNAs or pre-crRNAs in mammalian cells following plasmid transfection.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the CasPhi2 variant and the crRNA or pre-crRNA encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the CasPhi2 variant.

The present invention also includes the vectors and cells comprising the vectors.

Also provided herein are compositions and kits comprising the variants described herein. In some embodiments, the kits include the fusion proteins and a cognate guide RNA (i.e., a guide RNA that binds to the protein and directs it to a target sequence appropriate for that protein). In some embodiments, the kits also include labeled detector DNA, e.g., for use in a method of detecting a target ssDNA or dsDNA. Labeled detector DNAs are known in the art, e.g., as described in US20170362644; East-Seletsky et al., Nature. 2016 Oct. 13; 538 (7624): 270-273; Gootenberg et al., Science. 2017 Apr. 28; 356 (6336): 438-442, and WO2017219027A1, and can include labeled detector DNAs comprising a fluorescence resonance energy transfer (FRET) pair or a quencher/fluor pair, or both. The kits can also include one or more additional reagents, e.g., additional enzymes (such as RNA polymerases) and buffers, e.g., for use in a method described herein.

### Diagnostic Methods and Kits

Also provided herein are kits and methods for detecting a target DNA sequence in vitro. For example, provided herein are kits including any of the CasPhi2 variants described herein, a crRNA or pre-crRNA (e.g., SEQ ID NOs: 104-109) designed to be complementary to the target DNA sequence, and a single-stranded DNA whose cleavage generates a detectable signal (i.e., a fluorescent tag or label, such as DNase Alert (IDT)). In the so-called fluorophore quencher (FQ) assay, a fluorophore and a quencher are joined together by a short oligomer. These two components are separated by collateral ssDNA cleavage (in trans) of the CasPhi2 enyzme (or a variant thereof), once it binds to a specific target sequence. This separation leads to fluorescence^{18,19}. In the FQ assay, 100 nM CasPhi2 RNP can be used with the FQ probe and activator ssDNA (ssDNA detection) in cleavage buffer with 10 mM Hepes-Na pH 7.5, 150 mM KCl, 5 mM MgCl2, 10% glycerol, 0.5 mM TCEP. The reaction is incubated at 37° C. for up to 120 minutes at 37° C. with fluorescence measurements taken (plate reader) every 30 seconds^{16,20}. In some embodiments, the kit includes one or more crRNAs designed to recognize one or more target DNA sequences.

A method of detecting a target DNA sequence includes incubating the components of the kit, described above, with a DNA sample. Determining whether a detectable signal is generated indicates if the target DNA sequence is present in the DNA sample. In some embodiments, the kit includes two or more crRNAs designed to recognize two or more target DNA sequences.

CasPhi2 could be used with a fluorophore quencher assay to detect e.g. the DNA of an infectious agent, or a sequence in human DNA that contains a specific mutation.

### EXAMPLES

The invention is further described in the following examples, which do not limit the scope of the invention described in the claims.

### Methods

The following materials and methods were used in the Examples below.

Molecular cloning. A plasmid carrying the CasPhi2 gene¹⁵ was obtained from Addgene (plasmid no. 158801). All CasPhi2 mutants engineered in this study were cloned into a pCMV-T7 mammalian expression vector backbone derived from Addgene plasmid no. 112101 or 13277 by restriction digest with AgeI-HF and NotI-HF (New England Biolabs (NEB)) as follows. To clone the CasPhi2 mutants, DNA fragments with overhangs complimentary to the entry vector's backbone were first generated via PCR using Phusion high-fidelity DNA polymerase (NEB). The PCR fragments were separated by agarose gel electrophoresis and subsequently extracted using a Qiaquick PCR purification kit (Qiagen) and cleaned up with 2-3× paramagnetic beads (PMID 22267522). The purified PCR fragments were then inserted into a pCMV backbone generated as above, by Gibson assembly using Gibson mix (PMID 19369495) at 50° C. for 1 h and the reaction mix was used to transform chemically competent Escherichia coli XL1-Blue (Agilent).

The gRNAs used in this study were generated by annealing oligos for the spacer to form dsDNA (95° C. for 5 min, cool to 10° C. at −5° C./min) with complementary overhangs to the BsmBI-digested crRNA and pre-crRNA entry vectors, that were previously generated using BPK1520 (65777) as a template (pUC19-U6 backbone, digested with BsmbI and HindIII-HF).

All crRNAs used in this study were
of the form
SEQ-ID No. 104
5′-(G)CAACGAUUGCCCCUCACGAGGGGA
C-N₁₂₋₂₄-U₁₋₈,
All pre-crRNAs used in this study were
of the form
SEQ-ID No. 105
5′-(G)GUCGGAACGCUCAACGAUUGCCCCUCA
CGAGGGGAC-N₁₂₋₂₄-U₁₋₈,

The G in parentheses 5′ of the direct repeat (DR) sequences with both crRNA and pre-crRNA architectures represents an additional optional 5′G that can be added to enhance expression from the U6 promoter in a DNA-based expression vector. Also see FIG. 1D for a detailed depiction of the crRNA and pre-crRNA architectures in DNA expression vectors.

All plasmids used in this study were purified by Qiagen Mini/Midi Plus kits.

Cell culture. STR-authenticated HEK293T cells (CRL-3216, ATCC), K-562 cells (CCL-243), and U2OS cells (similar match to HTB-96; gain of no. 8 allele at the D5S818 locus) were used in this study. HEK293T and U20S cell lines were cultured in Dulbecco's modified Eagle medium (Gibco) supplemented with 10% FBS and 50 units/ml penicillin and 50 μg/ml streptomycin, while U2OS cells were supplemented with an additional 1% GlutaMAX (all from Gibco). K562 cells were grown in Roswell Park Memorial Institute (RPMI) 1640 Medium (Gibco) with 10% FBS, supplemented with 1% pen-strep and 1% GlutaMAX (Gibco). Cells were grown at 37° C. with 5% CO₂ and upon reaching 80% confluency were passaged into new medium (every 2-3 days). Cell culture supernatants were tested for mycoplasma contamination every 4 weeks with the MycoAlert PLUS mycoplasma detection kit (Lonza), and all results were negative for the duration of this study. For experiments with human induced pluripotent stem cell (hiPSC)-derived iCell Cardiomyocytes (obtained from Cellular Dynamics/Fujifilm, item 11713), plating medium (Cellular Dynamics) was thawed overnight at 4° C. before thawing the cells according to the manufacturer's recommendations. After resuspension and counting on a Luna-FL Cell Counter (Logos Bio), 2.5×10⁴ cells were seeded in 100 μL plating medium per well of a 96-well plate which had been coated with 0.1% gelatin for 4 hours. Maintenance medium (Cellular Dynamics) was thawed overnight at 4° C. 24h before use, followed by equilibration at 37° C. Cells were washed with maintenance medium 48h post-seeding and plating medium was replaced with 90 μL maintenance medium per well (replaced every other day). Cells were maintained at 37° C. under 5% CO2.

Transfections and Electroporations. HEK293T cells were seeded for transfection in 96-well flat-bottom cell culture plates (Corning) at 1.25×10+ cells in 92 μL growth medium/well. After 18-24 h incubation, the cells were transfected with plasmid DNA (for DNA cleavage: 30 ng WT-CasPhi2 or CasPhi2 variant, 10 ng pre-crRNA or crRNA; for base editing: 30 ng CasPhi2-BE, 10 ng crRNA;) using 0.3 μL TransIT-X2 lipofection reagent (Mirus) and 9 μL of Opti-MEM (Gibco) per well. For split base editor experiments, 40 ng total plasmid DNA (10 ng gRNA, 15 ng dCasPhi(D394A)(17aa), and 15 ng TadA8e) or 70 ng total plasmid DNA (10 ng gRNA, 30 ng dCasPhi(D394A)(17aa), and 30 ng TadA8e) were used. For HDR experiments in HEK293T cells, 3.5×10⁴ HEK293T cells seeded into 48-well plates were transfected 16-24 hours later with 100 ng total plasmid (75 ng CasPhi2-17aa, 25 ng crRNA) with or without (negative control) 1.5 pmol single stranded alt-R HDR oligos (IDT), 26 μL Opti-MEM and 0.78 μL of Transit-X2. HDR oligos were 83 bp long with 40 bp homology arms encoding ATG insertions at positions 9, 11, or 13, and PAM disrupting mutations.

For U2OS cells, 4×10⁶ cells were seeded into a 15-cm dish (Corning) in 15 ml growth medium. After 18-24 h of incubation, the cells (2×10⁵/sample) were electroporated with 1000 ng of total plasmid DNA (750 ng CasPhi2 or CasPhi2 variant, 250 ng crRNA) using the SE cell Line Nucleofector X Kit (Lonza) according to the manufacturer's protocol and plated in 500 μL of cell culture medium in 24-well flat-bottom plates (Corning). For K562 cells, 4×10⁶ cells were seeded into a 15-cm dish (Corning) in 15 ml growth medium. After 18-24 h of incubation, the cells (2×10⁵/sample) were electroporated with 1000 ng of total plasmid DNA (750 ng CasPhi2 or CasPhi2 variant, 250 ng crRNA) using the SF cell Line Nucleofector X Kit (Lonza) according to the manufacturer's protocol and plated in 500 μL of cell culture medium in 24-well flat-bottom plates (Corning).

iCell hiPSC-derived cardiomyocytes (Cellular Dynamics/Fujifilm) were transfected using Transit-LT1 transfection reagent (Mirus) on days 5, 6, and 7 post-thawing, using 150 ng of plasmid DNA from CasPhi2 variants (WT and T355R-D679K (double-mutant, DM) with GenScript Optimum codon optimization) and 50 ng of crRNA, as well as 9 μL Opti-MEM (Gibco) and 0.6 μL Transit-LT1 per well. Maintenance medium was replaced 3h pre-transfection and 24h post-transfection. After transfection or electroporation, cells were incubated at 37° C. under 5% CO₂ for 72 h before isolation of genomic DNA (gDNA).

DNA extraction. Cells were washed with 1×PBS (Gibco) and subsequently lysed with 43.5 μL gDNA lysis buffer (100 mM Tris-HCl (pH 8), 200 mM NaCl, 5 mM EDTA, 0.05% SDS), 1.25 μL 1 M DTT (Sigma), and 5.25 μL Proteinase K (800 U/ml, NEB) per well for HEK293T cells and 174 μL lysis buffer, 5 μL DTT, and 21 μL Proteinase K per well for U2OS cells. Cells were lysed overnight at 55° C. with shaking (HT Indors Multitron) at 500 rpm, and the gDNA extracted from the lysate with 2× paramagnetic beads (PMID 22267522). The DNA bound to the beads was washed three times with 70% ethanol using a Biomek FX^{P} Laboratory Automation Workstation (Beckman Coulter), and eluted in 25-75 μL 0.1× EB (Qiagen).

Library preparation for targeted amplicon sequencing. The concentrations of the extracted gDNA were determined with a Qubit4 fluorometer and dsDNA HS Assay Kit (Thermo Fisher). The amplicon library for sequencing was generated in a 2-PCR process where the sequence of interest was amplified while adding Illumina adapter sequences (PCR1) and subsequently unique Illumina barcodes were attached (PCR2). In PCR1, 5-20 ng of gDNA was used to amplify the genomic sequence of interest using primers containing Illumina-compatible adapter sequences using Phusion DNA polymerase (NEB) under the following reaction conditions: 98° C. for 2 min, followed by 30-35 cycles of 98° C. for 10 s, 68° C. for 12 s, and 72° C. for 12 s, and a final 72° C. extension for 10 min. The amplicons were purified with 0.7×paramagnetic beads (PMID 22267522), eluted in 30 μL 0.1×EB (Qiagen), and measured using the Quantifluor dsDNA quantification system (Promega) on a Synergy HT microplate reader (BioTek; set to 485/528 nm). To allow for more samples to be sequenced using the same barcode, PCR1 amplicons from non-overlapping genomic sequences from samples generated with the gene editor were occasionally pooled before PCR2, based on the concentration. Unique Illumina-compatible barcodes were added to the PCR1 amplicons in PCR2 (based on NEBnext E7600 barcodes as well as custom barcodes) using Phusion DNA polymerase (NEB) and 50-200 ng of PCR1 product per sample or pool. The reaction conditions were as follows: 98° C. for 2 min, 5-10 cycles of 98° C. for 10 s, 65° C. for 30 s, and 72° C. for 30 s, followed by a 72° C. extension for 10 min. The PCR2 products were purified with 0.7× paramagnetic beads, quantified using the Quantifluor system (Promega), and pooled based on the concentrations to ensure that all samples are represented equally in the final library. The final pool was cleaned once more with 0.6×paramagnetic beads to remove any residual primer-dimers and primers. The library of amplicons was then sequenced using Illumina Miseq kits or Miseq micro kits (Miseq Reagent Kit v2; 300 cycles, 2×150 bp, paired-end). FASTQ files were downloaded via BaseSpace (Illumina) for demultiplexed sequencing data analysis.

Next generation sequencing analysis. Amplicon sequencing data were analyzed using CRISPResso2 (PMID 30809026) in batch mode using Base Editor Output mode. Indel quantification data were taken from the CRISPResso output table labeled ‘CRISPRessoBatch_quantification_of_editing_frequency.txt.’ The indel frequencies reported around the cut site using the window parameters (-wc-1-w 6) were calculated as follows: ((‘insertions’+‘deletions’−‘insertions and deletions’)/‘reads aligned’)*100.

Gene activation experiments. HEK293T cells were transfected with dCasPhi2 (D394A)-VPR, dCasPhi2-DM (D394A)-VPR, or dCasPhi2-17AA (D394A)-VPR plasmids (375 ng) and single or pooled Casphi crRNA plasmids (125 ng). 24 hours prior to transfection, HEK293T cells (6.25×10⁴) were seeded in 24-well plates and then lipofected with the plasmids using 3 μl of TransIT-X2 (Mirus Bio). Biological replicates are independent transfections on separate days or on same days with cells that have different passage numbers. 72 hours post-transfection, total RNA was extracted from the cells using the NucleoSpin RNA Plus Kit (Clontech) and 250 ng of purified RNA was used for cDNA synthesis using High-Capacity RNA-to-cDNA Kit (ThermoFisher). The cDNA was used for quantitative PCR (qPCR) using Fast SYBR Green Master Mix (ThermoFisher) with the gene-specific primers (Table 7) in 384-well plates on a LightCycler 480 (Roche) with the following program: initial denaturation at 95° C. for 20 seconds(s) followed by 45 cycles of 95° C. for 3 s and 60° C. for 30 s. Since Ct values fluctuate for transcripts expressed at very low levels, values greater than 35 were considered as 35, and used as the baseline Ct value. Gene expression levels were normalized to HPRT1 and calculated relative to that of the negative controls (dCasPhi2 (D394A)-VPR and/or VPR fusions with newer dCasPhi2 (D394A) variants and non-targeting gRNA plasmids). HPRT1 qPCR control was independently assayed for each sample. Frequency, mean, and standard error of the mean were calculated using GraphPad Prism 8.

TABLE 7
Forward and Reverse RT-qPCR primers
for CD69 and IL2RA

| | | | |
|---|---|---|---|
| CD69 | RT-qPCR | Forward | GCTGGACTTCAGCCCAAAATGC |
| CD69 | RT-qPCR | Reverse | AGTCCAACCCAGTGTTCCTCTC |
| IL2RA | RT-qPCR | Forward | GAGACTTCCTGCCTCGTCACAA |
| IL2RA | RT-qPCR | Reverse | GATCAGCAGGAAAACACAGCCG |

### Example 1: CasPhi2 Gene Editing Activity is Neither Robust Nor Efficient in Human Cells

Wild-type (WT) CasPhi2 was previously reported to possess gene editing activity in human cells but this conclusion was based solely on reduced expression of an integrated EGFP gene with no confirmation that CasPhi2-induced gene edits were successfully induced in the reporter coding sequence¹⁵. To directly assess whether WT CasPhi2 could induce gene editing in human cells, we tested this nuclease with two different GFP-targeted crRNAs (crRNA 6 and crRNA 8) previously reported to reduce GFP reporter gene expression by 10-30% in human cells in that earlier published study¹⁵. To do this, we co-transfected a HEK293-GFP cell line (harboring an integrated GFP reporter gene) with plasmids expressing WT CasPhi2 nuclease and crRNA6 or crRNA8 and assessed the percentage of GFP-negative cells at 72 hours post-transfection using flow cytometry. We observed ˜19-20% GFP-negative cells with each of the two GFP-targeted crRNAs (FIG. 1A), a result similar to the ˜10-30% reported in the previously published characterization of these crRNAs with WT CasPhi215. A “no treatment” negative control yielded ˜2.6% GFP-negative cells while a SpCas9 positive control using a previously described FYF gRNA yielded ˜60% GFP-negative cells (FIG. 1A). However, we also observed ˜14.5% GFP-negative cells in negative controls in which we only transfected plasmid expressing either WT CasPhi2 or SpCas9 alone (i.e., without a crRNA or gRNA) while transfection of plasmid expressing crRNA8 alone led to low frequencies of GFP-negative cells (˜3.7%) similar to what was observed with the no treatment negative control (FIG. 1A). Taken together, these results suggest that decreased GFP expression induced by WT CasPhi2 in human cells is most likely not primarily due to targeted gene editing (or targeted DNA binding) by co-expressed crRNAs but instead much of the observed reduction in GFP activity can be attributed to transfection of just the CasPhi2 expression plasmid (with GFP repression occurring by an an as-yet-unknown mechanism). Consistent with this, targeted amplicon sequencing using NGS of the targeted region of GFP in our transfected HEK293-GFP cells revealed very low indel frequencies of <5% or <10% induced by WT CasPhi2 with crRNA6 or crRNA8, respectively (FIG. 1B), but ˜60% with SpCas9 and the FYF gRNA (FIG. 1B). Based on these results, we conclude that the crRNA-targeted gene editing activities of WT CasPhi2 enzyme are substantially lower in human cells than previously suggested by the GFP disruption assay. Consistent with this, while this work was in progress, others have also demonstrated the low efficiencies of WT CasPhi2 nuclease in human cells²¹.

To more comprehensively assess the gene editing activity of WT CasPhi2 in human cells, we tested this nuclease with a series of 19 crRNAs targeting various endogenous gene sequences in HEK293T cells. Strikingly, we observed detectable gene editing (defined as >1% indels) with only one of the 19 crRNAs tested: the VEGFA site 3 crRNA, which induced indels with only a modest frequency of ˜5% (FIG. 1C). To test whether using pre-crRNAs (which have a longer direct repeat sequence than processed crRNA sequences) might increase editing efficiencies (FIG. 1D), we targeted 17 of the same 19 spacer sequences using pre-crRNAs. This experiment showed that only one of these 17 pre-crRNAs induced detectable indels at frequencies >1% but at this target site (VEGFA site 3 again) the mean editing frequency observed was only ˜3% (FIGS. 1E & 1F). Although this editing frequency was lower than the ˜5% we observed using a crRNA targeting the same spacer (FIG. 1C above), to our knowledge these results provide the first demonstration that a pre-cRNA can function to direct CasPhi2 nuclease to a target site in human cells.

### Example 2: Engineering CasPhi2 Variants

Overview of Multi-Stage Engineering Strategy for Creating CasPhi2 Variants with Higher Activities in Human Cells

Given the low and non-robust activity of WT CasPhi2, we next sought to determine if we could use a combination of rational engineering and mutation shuffling to create CasPhi2 variants with higher activities in human cells. CasPhi2 shows efficient cleavage function in vitro¹⁵ suggesting that its enzymatic cleavage activity is robust and therefore not likely to be the rate limiting step for its gene editing activity in human cells. We hypothesized that perhaps instead the affinity of this enzyme for DNA in human cells might be insufficient to stabilize its binding to DNA so that gene editing can occur. We further reasoned that increasing CasPhi2 affinity for its target site might be accomplished by introducing positively charged amino acids at CasPhi2 residues that reside close to the target DNA or crRNA. We also envisioned that we might combine any single amino acid substitutions that showed higher activity together to create and identify multi-mutation CasPhi2 variants with even more improved gene editing activities in human cells.

Our efforts to create higher activity CasPhi2 variants therefore consisted of three stages. In Stage I, because we did not have structural information available to us when we performed these experiments, we built and used homology alignments to guide the choice of individual CasPhi2 residues to convert to positively charged amino acids. Screening of 20 single amino acid substitution variants yielded two mutations that increase CasPhi2 activity in human cells. We combined these two mutations to create a CasPhi2 double mutant (CasPhi2-DM) that exhibited consistently higher activity than WT CasPhi2 as a gene-editing nuclease in human cells. In Stage II, we used structural information about WT CasPhi2 (that was published while we were pursuing our Stage I efforts) to identify 159 additional residues for mutation. We added mutations at each of these positions to CasPhi2-DM and then screened the gene editing activities of these triple mutation variants in HEK293T cells. This large-scale screening identified 24 additional residues where mutation further increased the gene editing activity of CasPhi2-DM in human cells. Lastly, in Stage III, we generated a large series of CasPhi2-DM-derived variants that harbored various combinations of the 24 activity-enhancing mutations we identified in Stage II together with the two mutations in the CasPhi2-DM. These experiments yielded multiple CasPhi2 variants harboring four to 17 amino acid substitutions that showed substantially improved and highly robust activities in human cells.

### Engineering Higher Activity CasPhi2 Variants—Stage I (Model-Guided Mutagenesis)

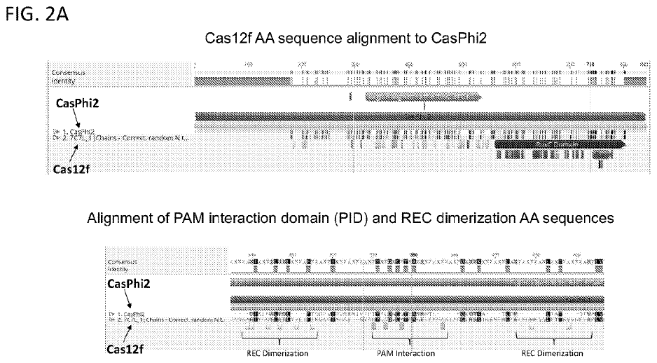

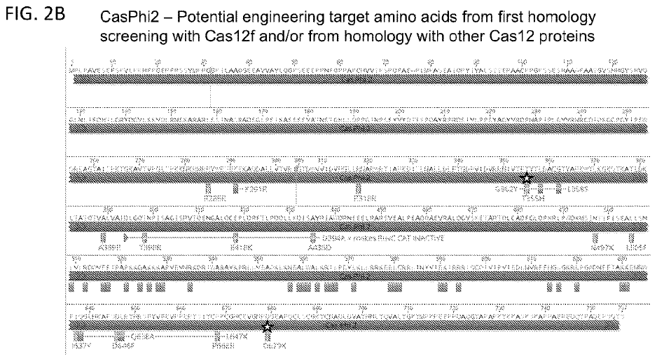

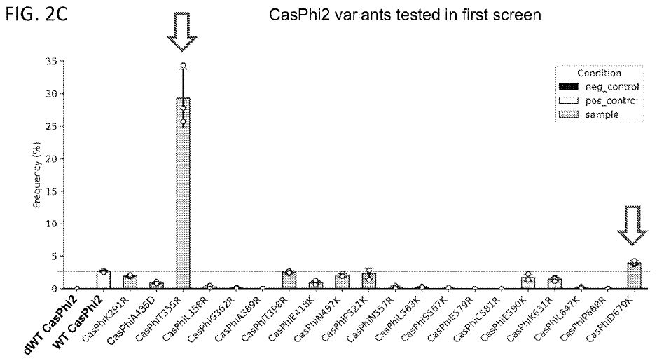

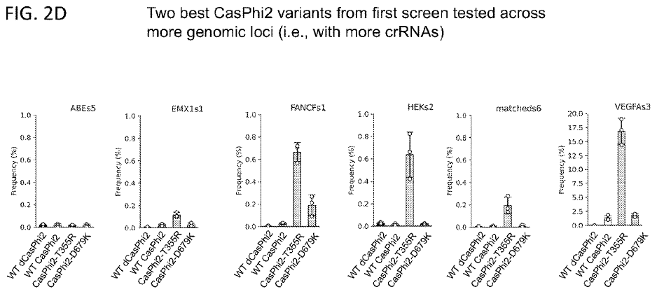

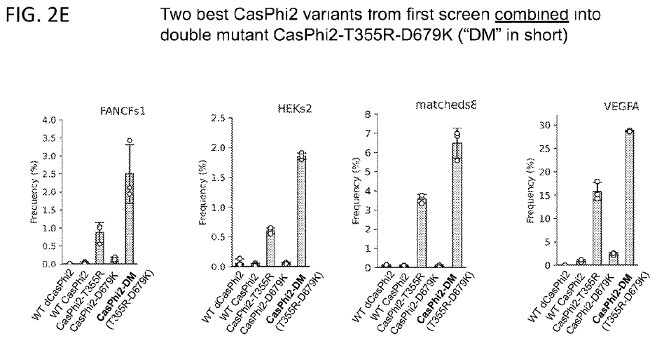

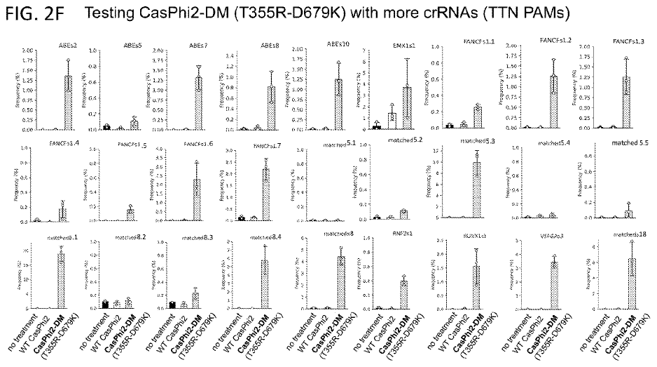

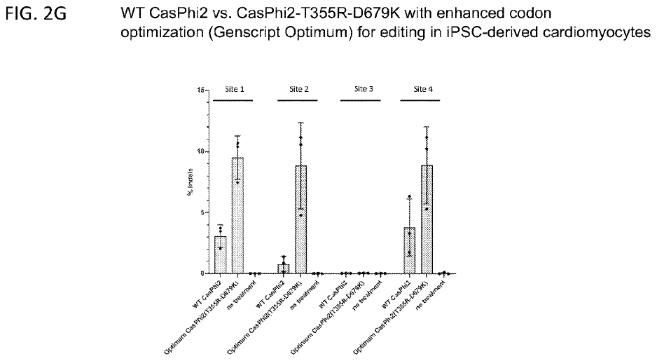

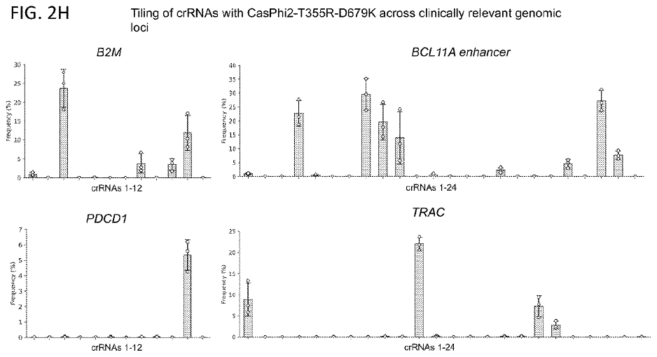

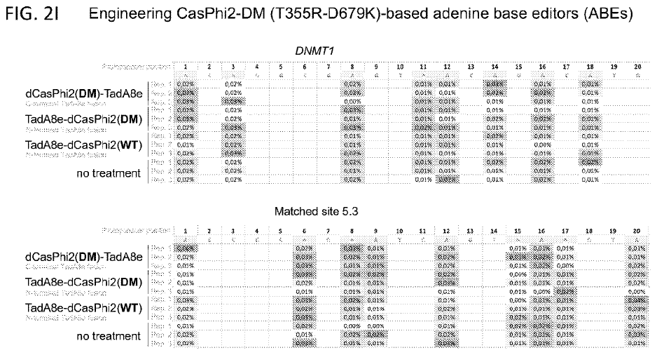

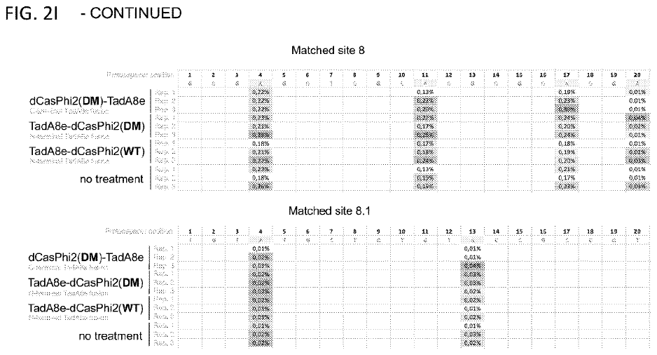

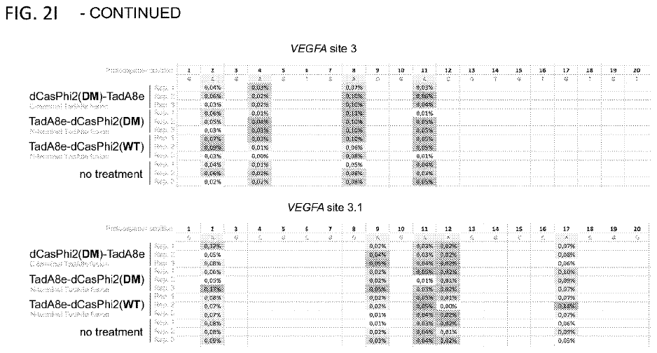

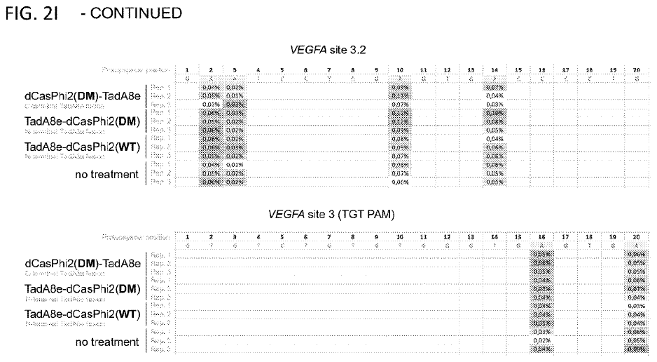

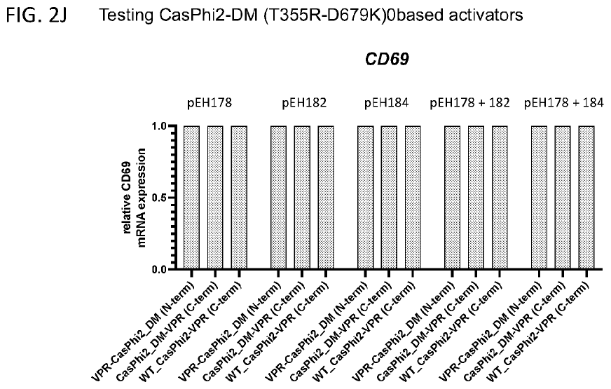

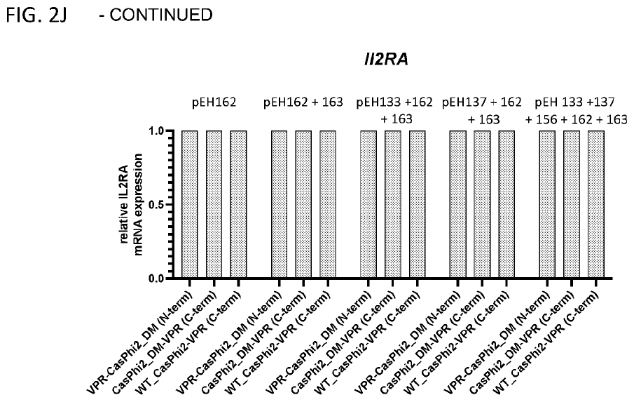

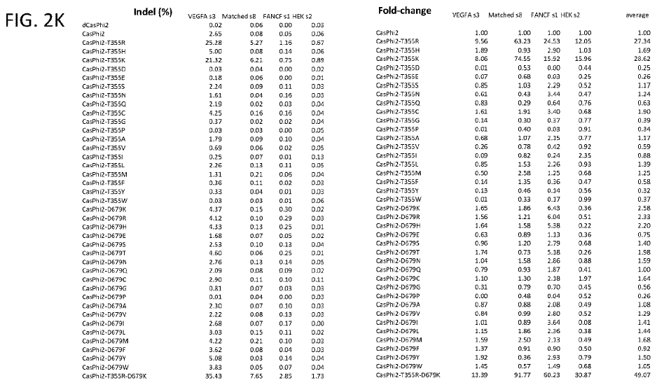

As noted above, because no structural information was available when we began our CasPhi2 engineering efforts, we instead used homology alignments to guide our mutagenesis efforts. To accomplish this, we used type V systems from the Cas12f family (also known as Cas14⁸), which are the prokaryotic CRISPR proteins most closely related to CasPhi2 despite having overall relatively low amino acid (AA) sequence homology¹⁵. We aligned amino acid sequences of both enzymes and could detect a number of functionally relevant regions with AA homology, e.g., the RuvC domain, as well as REC dimerization and PAM interaction domains (FIG. 2A). Based on these alignments, and alignments to other WT or engineered Cas12 enzymes, such as enAsCas12a or BhCas12b, we selected 20 residues in CasPhi2 that aligned with Cas12f residues predicted by our model to be present in the PAM interacting domain, in a TNB/disordered domain, in or near the catalytic center, or in the RuvC domain (Table 7, FIG. 2B)^{12,22,23}. We created a series of single mutation CasPhi2 variants bearing positively charged residues (R or K) at 19 of these 20 positions and a negative charge substitution at the A435 position (A435D) to mimic a D510 residue present in the catalytic center of a Cas12f protein¹² (Table 8, FIG. 2B).

TABLE 8
Cas12 alignments with CasPhi2 to engineer variants for screen no. 1

| | | | homologous | | | |
|---|---|---|---|---|---|---|
| | CasPhi2 | | AA (and/or | Cas with | | |
| | candidate | | mutation | homologous | Function or | |
| | amino | CasPhi2 | change) in | AA based | location of | |
| | acid | mutant | other Cas & | on AA | homologous AA | Ref. |
| # | position | tested | variants | alignments | in other Cas | (PMID) |
| 1 | R318 | | E174R | enAsCas12a | relaxed PAM, | 30742127 |
| 2 | R285 | | S542R | | increased | |
| 3 | K291 | K291R | K548R | | efficiencies | |
| 4 | H614 | | E837G | BhCas12b | increase | 30670702 |
| | | | | | protein | |
| | | | | | flexibility | |
| | | | | | (helped | |
| | | | | | increase DNA | |
| | | | | | cleavage | |
| | | | | | activity at 37 C.) | |
| 5 | K631 | K631R | K846R | | may help pull | |
| | | | | | target-strand | |
| | | | | | toward RuvC | |
| 6 | P668 | P668R | S893R | | may help pull | |
| | | | | | target-strand | |
| | | | | | toward RuvC | |
| 7 | T355 | T355R | H139 | Cas12f | PAM | 33333018 |
| | | | | | interaction | |
| 8 | L358 | L358R | S142 | | PAM | |
| | | | | | interaction | |
| 9 | G362 | G362R | Y146 | | PAM | |
| | | | | | interaction | |
| 10 | no align. | | A156 | | PAM | |
| | | | | | interaction | |
| 11 | D679 | D679K | K491 | | TNB/disordered | |
| | | | | | region | |
| 12 | A435 | A435D | D510 | | Helps form | |
| | | | | | catalytic center | |
| 13 | A389 | A389R | | | R flanking | 30670702 |
| | | | | | catalytic center | |
| | | | | | in Cas12b | |
| 14 | T398 | T398R | | | R flanking | |
| | | | | | catalytic center | |
| | | | | | in Cas12b | |
| 15 | E418 | E418K | K415 | | near catalytic | 33333018 |
| | | | | | center E422. | |
| | | | | | It's a K in | |
| | | | | | Cas12f | |
| 16 | N497 | N497K | K330 | | RuvC | |
| 17 | L505 | | F341 | | RuvC | |
| 18 | S509 | | S345 | | RuvC | |
| 19 | R512 | | D348 | | RuvC | |
| 20 | F516 | | F352 | | RuvC | |
| 21 | F517 | | H353 | | RuvC | |
| 22 | P521 | P521K | K356 | | RuvC | |
| 23 | G524 | | F359 | | RuvC | |
| 24 | K526 | | R361 | | RuvC | |
| 25 | K528 | | R363 | | RuvC | |
| 26 | A529 | | I364 | | RuvC | |
| 27 | R535 | | K367 | | RuvC | |
| 28 | N557 | N557R | R373 | | RuvC | |
| 29 | A559 | | G375 | | RuvC | |
| 30 | L560 | | H376 | | RuvC | |
| 31 | W561 | | G377 | | RuvC | |
| 32 | L563 | L563K | K379 | | RuvC | |
| 33 | R565 | | K381 | | RuvC | |
| 34 | T566 | | L382 | | RuvC | |
| 35 | S567 | S567K | K383 | | RuvC | |
| 36 | L571 | | T386 | | RuvC | |
| 37 | R576 | | K391 | | RuvC | |
| 38 | E579 | E579R | R394 | | RuvC | |
| 39 | L580 | | F395 | | RuvC | |
| 40 | C581 | C581R | R396 | | RuvC | |
| 41 | E590 | E590K | K397 | | RuvC | |
| 42 | K591 | | K398 | | RuvC | |
| 43 | R594 | | E401 | | RuvC | |
| 44 | S616 | | L424 | | RuvC | |
| 45 | L620 | | K428 | | RuvC | |
| 46 | E632 | | R438 | | RuvC | |
| 47 | I637 | | Y445 | | RuvC | |
| 48 | Q638 | | A446 | | RuvC | |
| 49 | D646 | | F454 | | RuvC | |
| 50 | L647 | L647K | K455 | | RuvC | |

We next screened these 20 different CasPhi2 single mutation variants for their nuclease-mediated gene editing activities in human cells. We performed these experiments using the VEGFA site 3 crRNA (VEGFA site 3) that had previously shown some, albeit very low, gene editing activity in human cells when tested with WT CasPhi2 (Example 1 above). We co-transfected HEK293T cells with plasmids encoding the VEGFA site 3 crRNA with each of the 20 single mutation CasPhi2 variants, WT CasPhi2, or a “dead” CasPhi2 mutant bearing a D394A (dWT CasPhi2 (D394A)) that inactivates catalytic nuclease activity as a negative control and used targeted amplicon sequencing to assess the frequency of indels introduced at the target site (see Methods section above). Two of the 20 single mutation CasPhi2 variants (T355R and D679K) induced increased frequencies of indels relative to WT CasPhi2 with the VEGFA site 3 crRNA (FIG. 2C). Testing of these two CasPhi2 variants with additional crRNAs targeting six different endogenous human genes detected substantial increases in editing frequencies with CasPhi2-T355R at 5 of the 6 sites and with CasPhi2-D679K at one of the 6 sites D679K (FIG. 2D). Testing gene editing efficiencies of CasPhi2 with mutations at residues T355 and D679 other than T355R or D679K, respectively, yielded comparable gains in gene editing efficiencies (e.g., with T355K (compared to T355R), as well as with D679R, D679H, and D679T (compared to D679K)) (FIG. 2K).

We next combined the T355R and D679K mutations to create a CasPhi2 double-mutant (CasPhi2-DM) variant and found that CasPhi2-DM outperformed both CasPhi2-T355R and CasPhi2-D679K when tested with four different crRNAs targeting endogenous genes in HEK293T cells (FIG. 2E). We performed further side-by-side testing of CasPhi2-DM and WT CasPhi2 in HEK293T cells with a larger set of 27 additional crRNAs targeting various endogenous human genes and observed substantial gains in gene editing frequencies at 18 of the 27 sites (FIG. 2F). We also tested CasPhi2-DM and WT CasPhi2 with sets of crRNAs targeted to two endogenous gene loci (VEGFA site 3 and matched site 8) in which we systematically varied the spacer sequence length targeted from 12 to 24 nucleotides (nts) and found that CasPhi2-DM showed activity with spacers ranging from 16 to 24 nts at both target sites (FIG. 3A); by contrast, WT CasPhi2 showed very low activity with spacers ranging from 18-24 nts on the VEGFA site 3 target site and no activity with all spacer lengths tested at matched site 8 (FIG. 3A). These results suggest that crRNAs with spacer sequence lengths shorter and longer than 20 nts are also capable of directing CasPhi2-DM gene editing activity to target sites in human cells. Notably, crRNAs with spacer lengths of 18 nts exhibit higher mean editing frequencies than those with spacer lengths of 20 nts at the two target sites we tested (FIG. 3A).

An important and potentially advantageous property of the CasPhi2 system is that it can cleave tandem arrays of its own pre-crRNAs to yield multiple crRNAs, a feature that simplifies the multiplex nuclease-mediated editing of target genes¹⁵. To test whether CasPhi2-DM (like WT CasPhi2, in vitro) was able to process pre-crRNAs in mammalian cells, we constructed plasmids designed to express an array of pre-crRNAs targeting two or three different target sites (VEGFA site 3, matched site 8, FANCF site 1) from a human U6 promoter. Multiplex pre-crRNA assays consisted of 36nt pre-crRNA direct repeats (DRs) and 20nt spacers (FIG. 3B and Methods, see section above). When tandem arrays of two or three pre-crRNAs were co-expressed with CasPhi2-DM in HEK293T cells, we observed editing at either both or all three target sites, albeit with efficiencies lower than those obtained when co-expressing crRNAs designed to target each of these three sites individually (FIG. 3C). Analogous experiments performed with WT CasPhi2 did not show evidence of multiplex editing but editing frequencies induced for the matched site 8 and FANCF site 1 target sites was not detectable even when each crRNA was expressed individually with WT CasPhi2 (FIG. 3C). We conclude that CasPhi2-DM is also capable of processing its own crRNAs from a larger tandem RNA transcript in mammalian cells.

To explore whether CasPhi2-DM might also function for nuclease-mediated gene editing in other non-cancer human cells, we also tested it side-by-side with WT CasPhi2 in clinically relevant human iPSC-derived cardiomyocytes. Using crRNAs targeted to four different endogenous gene loci, we observed that both CasPhi2-DM and WT Cas-Phi2 induced modest gene editing (mean editing frequencies of <10%) at three of the four sites we tested (FIG. 2G); however, CasPhi2-DM consistently outperformed WT CasPhi2 across all three of these target sites (FIG. 2G). Based on these results, we conclude that CasPhi2-DM can function to induce gene editing in non-cancer cell lines and not just in cancer cell lines like HEK293T cells.

We assessed the robustness of the CasPhi2-DM variant for nuclease-mediated gene editing by tiling larger series of crRNAs across four clinically relevant gene targets in human cells. To accomplish this, we screened panels of 12 crRNAs each for the B2M and PDCD1 genes and panels of 24 crRNAs each for the TRAC gene and the erythroid-specific transcriptional enhancer of the BCL11A gene in HEK293T cells (FIG. 2H). For the B2M gene, five of the 12 crRNAs we tested showed gene editing with CasPhi2-DM, with one yielding >10% and another yielded >20% mean indel frequencies at their target sites (FIG. 2H). For the PDCD1 gene, one of the 12 crRNAs tested with CasPhi2-DM showed gene editing activity, yielding mean indel frequency of ˜5% (FIG. 2H). For the TRAC gene, four of the 24 crRNAs yielded gene editing activities with CasPhi2-DM; two of the crRNAs induced >5% and one induced >20% mean indel frequencies (FIG. 2H). Finally, at the BCL11A enhancer, 11 of the 24 crRNAs tested showed gene editing activity with CasPhi2-DM, one crRNA inducing >5%, two crRNAs inducing >10%, and three crRNAs inducing 20-30% mean indel frequencies (FIG. 2H).

### Example 3: Characterization of CasPhi2-DM-Based Fusion Proteins for Base Editing and Epigenetic Editing Activities in Human Cells

We tested whether we could construct a CRISPR base editor using our CasPhi2-DM variant. To do this, we created two potential base editors in which we fused the adenine deaminase domain TadA8e²⁴ to the N- or C-terminus of “dead” CasPhi2-DM bearing a D394A mutation that inactivates its nuclease activity (dCasPhi2-DM (D394A)). We also constructed corresponding fusions using dead WT CasPhi2 (WT dCasPhi2 (D394A)). We then tested these fusions in HEK293T cells with eight crRNAs that we had previously shown induced varying frequencies of gene editing at their target sites with WT CasPhi2 and/or CasPhi2-DM in these same cells (FIGS. 1C and 2F). However, we did not detect any A to G base editing that was >1% with any of the fusions we tested (FIG. 2I).

We also tested whether the CasPhi2-DM variant could be used to construct active epigenetic editors. Specifically, we sought to construct fusion proteins capable of functioning as targetable transcriptional activators. To assess this possibility, we constructed expression plasmids encoding fusion proteins consisting of the strong synthetic VPR transcriptional activation domain fused to the N- or C-terminus of dCasPhi2-DM (D394A) and the C-terminus of dWT CasPhi2 (D394A). We co-transfected each of these plasmids with a single plasmid or pools of plasmids encoding single individual crRNAs or combinations of 2-5 crRNAs targeted to sites in the promoters of the human IL2RA and CD69 genes (each of these crRNAs had individually induced indel mutations at their respective on-target sites when tested with CasPhi2-DM nuclease). We then assessed expression of the target genes relative to negative control cells using quantitative RT-PCR (see Methods section above) but we failed to observe transcriptional activation with any of the individual or pooled combinations of crRNAs (FIG. 2J).

### Example 4: Engineering Higher Activity CasPhi2 Variants-Stage II (Structure-Guided Mutagenesis)

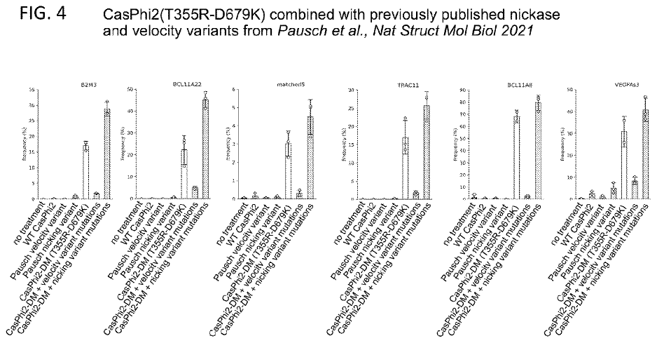

To attempt to further improve the gene editing activity of our CasPhi2-DM variant in human cells, we performed additional mutagenesis guided by cryo-EM structures of WT CasPhi216 that were published while we were conducting our Stage I engineering work. Using the WT CasPhi2 structure (PDB structure 7LYS), we identified 262 amino acid residues (present in various domains of the protein) that were less than 2.5 or 5 angstroms away from DNA or RNA present in the structure (Table 2). 156 of these 262 positions were not arginine or lysine and therefore were candidates for targeted mutation to positively charged residues to increase gene editing activity. In addition, we chose three additional positions within CasPhi2 for mutation (E159, D167, and E168). We selected these three residues (E159, D167, and E168) because we had found that the addition of five alanine substitution mutations (E159A, S160A, S164A, D167A, E168A; reported as a “nickase” CasPhi2 in the publication describing the CasPhi2 structure¹⁶ to the CasPhi2-DM variant modestly increased its human cell gene editing activity across six different target sites in HEK293T cells (FIG. 4) and these three residues were not present among the 167 nucleic acid-proximal residues we identified from our structural analysis (whereas residues S160 and S164 had been identified by our analysis) (Table 9).

TABLE 9
Structure-based identification of single CasPhi2 amino acid residues based on proximity to any
nucleic acid (spacer, protospacer-adjacent motif (PAM), non-target strand (NTS), target-strand
(TS), direct repeat (DR)) in the cryo-EM structure PDB 7LYS. Second row shows distances from
individual residue to the respective nucleic acid designated in the column in Angstrom (A).
Listed residues were either within 5 or 2.5 A distance from the respective nucleic acid.

| # | SPACER | | | PAM | | | NTS | | | TS | | | DR | |
|---|---|---|---|---|---|---|---|---|---|---|---|---|---|---|
| A | 5 | 2.5 | # | 5 | 2.5 | # | 5 | 2.5 | # | 5 | 2.5 | # | 5 | 2.5 |
| 1 | F58 | F58 | 1 | F10 | | 1 | S8 | | 1 | K29 | K29 | 1 | P60 | |
| 2 | Q59 | | 2 | M28 | | 2 | F10 | F10 | 2 | R30 | R30 | 2 | P61 | |
| 3 | P60 | | 3 | K29 | | 3 | S11 | | 3 | K33 | K33 | 3 | K63 | |
| 4 | P61 | P61 | 4 | R30 | | 4 | L14 | L14 | 4 | Q59 | | 4 | C64 | |
| 5 | K63 | K63 | 5 | G32 | | 5 | K15 | K15 | 5 | P61 | | 5 | H65 | H65 |
| 6 | R139 | | 6 | K33 | | 6 | F18 | | 6 | Q127 | | 6 | R226 | R226 |
| 7 | V143 | | 7 | A36 | | 7 | P19 | | 7 | L131 | | 7 | I232 | |
| 8 | K146 | K146 | 8 | K104 | | 8 | R22 | R22 | 8 | D134 | | 8 | P233 | |
| 9 | R150 | | 9 | S105 | | 9 | S25 | | 9 | H135 | H135 | 9 | L234 | L234 |
| 10 | Q190 | Q190 | 10 | S106 | S106 | 10 | M28 | | 10 | G138 | | 10 | G235 | |
| 11 | P191 | | 11 | E107 | | 11 | K29 | K29 | 11 | R139 | | 11 | V236 | |
| 12 | P192 | P192 | 12 | V126 | | 12 | R30 | | 12 | D141 | | 12 | V237 | |
| 13 | G193 | G193 | 13 | Q127 | | 13 | G32 | | 13 | G142 | G142 | 13 | R238 | R238 |
| 14 | I194 | | 14 | N130 | | 14 | K33 | | 14 | V143 | | 14 | N239 | N239 |
| 15 | N195 | N195 | | | | 15 | L35 | | 15 | K145 | | 15 | R240 | R240 |
| 16 | P196 | | | | | 16 | A36 | | 16 | K146 | K146 | 16 | K245 | |
| 17 | S197 | | | | | 17 | K104 | | 17 | L149 | L149 | 17 | C247 | |
| 18 | Y199 | | | | | 18 | S105 | | 18 | R150 | | 18 | P248 | |
| 19 | W322 | | | | | 19 | S106 | S106 | 19 | K153 | | 19 | G249 | G249 |
| 20 | R323 | R323 | | | | 20 | E107 | | 20 | N195 | | 20 | Y250 | Y250 |
| 21 | V344 | | | | | 21 | S124 | | 21 | Y199 | | 21 | I251 | I251 |
| 22 | D346 | | | | | 22 | H125 | | 22 | Y201 | | 22 | P252 | |
| 23 | R348 | | | | | 23 | V126 | V126 | 23 | Q202 | Q202 | 23 | W254 | W254 |
| 24 | R349 | R349 | | | | 24 | Q127 | Q127 | 24 | F339 | | 24 | Q255 | |
| 25 | T353 | | | | | 25 | N130 | | 25 | T340 | T340 | 25 | R256 | |
| 26 | T355 | | | | | 26 | A156 | | 26 | G341 | G341 | 26 | A261 | |
| 27 | W440 | | | | | 27 | R157 | | 27 | D342 | D342 | 27 | I262 | |
| 28 | E444 | | | | | 28 | S160 | S160 | 28 | V344 | | 28 | S263 | |
| 29 | R448 | | | | | 29 | I161 | | 29 | T355 | | 29 | P264 | |
| 30 | F517 | | | | | 30 | S164 | | 30 | T357 | | 30 | K265 | |
| 31 | T518 | | | | | 31 | Q202 | | 31 | W440 | | 31 | T266 | |
| 32 | A520 | | | | | 32 | T203 | | 32 | S496 | | 32 | K268 | |
| 33 | R535 | | | | | 33 | I204 | | 33 | N497 | N497 | 33 | V270 | |
| 34 | T539 | | | | | 34 | R210 | | 34 | F517 | F517 | 34 | T271 | |
| 35 | R542 | | | | | 35 | R212 | | 35 | T518 | | 35 | V272 | |
| 36 | K545 | | | | | 36 | R303 | R303 | 36 | P519 | | 36 | P273 | |
| 37 | R547 | R547 | | | | 37 | I304 | | 37 | A520 | | 37 | G274 | G274 |
| 38 | L548 | | | | | 38 | Y364 | | 38 | P521 | | 38 | L275 | |
| 39 | Q553 | | | | | 39 | K367 | | 39 | K522 | K522 | 39 | S276 | S276 |
| 40 | K556 | | | | | 40 | W368 | W368 | 40 | V533 | | 40 | P277 | |
| 41 | N557 | | | | | 41 | T369 | | 41 | R535 | | 41 | K278 | |
| 42 | L560 | | | | | 42 | K371 | | 42 | K536 | K536 | 42 | K279 | |
| 43 | W561 | | | | | 43 | G372 | | 43 | R538 | | 43 | N280 | N280 |
| 44 | K564 | | | | | 44 | K373 | | 44 | T539 | | 44 | K281 | K281 |
| 45 | R575 | | | | | 45 | Q374 | | 45 | R542 | | 45 | R282 | R282 |
| 46 | R582 | R582 | | | | 46 | R659 | | 46 | L560 | | 46 | M283 | M283 |
| 47 | R611 | | | | | 47 | T712 | F10 | 47 | W561 | | 47 | R284 | |
| 48 | H614 | | | | | | | | 48 | K564 | | 48 | R285 | R285 |
| 49 | G615 | | | | | | | | 49 | R565 | R565 | 49 | Y286 | Y286 |
| 50 | S616 | | | | | | | | 50 | Y570 | | 50 | W287 | W287 |
| 51 | G617 | G617 | | | | | | | 51 | L571 | | 51 | K293 | K293 |
| 52 | R619 | | | | | | | | 52 | S574 | S574 | 52 | D296 | D296 |
| 53 | T628 | | | | | | | | 53 | E578 | | 53 | A297 | |
| 54 | A629 | | | | | | | | 54 | N609 | | 54 | L298 | |
| 55 | K630 | K630 | | | | | | | 55 | V610 | | 55 | D312 | |
| 56 | E632 | | | | | | | | 56 | R611 | | 56 | R314 | R314 |
| 57 | R634 | | | | | | | | 57 | R634 | | 57 | G315 | |
| 58 | Q638 | | | | | | | | 58 | Q638 | | 58 | L317 | |
| 59 | T649 | | | | | | | | 59 | G639 | | 59 | R318 | R318 |
| 60 | H650 | H650 | | | | | | | 60 | K642 | K642 | 60 | N319 | |
| 61 | R651 | | | | | | | | | | | 61 | R321 | R321 |
| | | | | | | | | | | | | 62 | W322 | W322 |
| | | | | | | | | | | | | 63 | R323 | |
| | | | | | | | | | | | | 64 | K328 | K328 |
| | | | | | | | | | | | | 65 | A435 | |
| | | | | | | | | | | | | 66 | A439 | |
| | | | | | | | | | | | | 67 | R442 | |
| | | | | | | | | | | | | 68 | E569 | |
| | | | | | | | | | | | | 69 | K572 | |
| | | | | | | | | | | | | 70 | L573 | |
| | | | | | | | | | | | | 71 | R575 | |
| | | | | | | | | | | | | 72 | R576 | R576 |
| | | | | | | | | | | | | 73 | E578 | |
| | | | | | | | | | | | | 74 | E579 | E579 |
| | | | | | | | | | | | | 75 | L580 | |
| | | | | | | | | | | | | 76 | R582 | R582 |
| | | | | | | | | | | | | 77 | R583 | R583 |
| | | | | | | | | | | | | 78 | N586 | |
| | | | | | | | | | | | | 79 | H650 | |
| | | | | | | | | | | | | 80 | R651 | |

TABLE 10
Subset of CasPhi2 residues from Table 2 that were selected as
candidates for engineering new CasPhi2 variants in engineering/screening
round 1. All variants are based on the DM variant (T355R-D679K).
“AA” designates residue in WT CasPhi2, “position”
designates residue position/number in the CasPhi2 protein, counting
from start codon/methionine (=position 1). New AA designates
what the respective WT AA residue is mutated to, e.g., S8 is
mutated to R8 (#1).

| | | | New |
|---|---|---|---|
| # | AA | position | AA |
| 1 | S | 8 | R |
| 2 | F | 10 | R |
| 3 | S | 11 | R |
| 4 | L | 14 | R |
| 5 | F | 18 | R |
| 6 | P | 19 | R |
| 7 | S | 25 | R |
| 8 | M | 28 | R |
| 9 | G | 32 | R |
| 10 | L | 35 | R |
| 11 | A | 36 | R |
| 12 | V | 44 | R |
| 13 | F | 58 | A |
| 14 | F | 58 | R |
| 15 | Q | 59 | K |
| 16 | P | 60 | R |
| 17 | P | 61 | R |
| 18 | C | 64 | R |
| 19 | H | 65 | R |
| 20 | S | 105 | R |
| 21 | S | 106 | R |
| 22 | E | 107 | R |
| 23 | S | 124 | R |
| 24 | H | 125 | R |
| 25 | V | 126 | R |
| 26 | Q | 127 | R |
| 27 | N | 130 | R |
| 28 | L | 131 | R |
| 29 | D | 134 | R |
| 30 | H | 135 | R |
| 31 | G | 138 | R |
| 32 | D | 141 | K |
| 33 | G | 142 | R |
| 34 | V | 143 | R |
| 35 | L | 149 | R |
| 36 | A | 156 | K |
| 37 | E | 159 | R |
| 38 | S | 160 | K |
| 39 | I | 161 | K |
| 40 | S | 164 | K |
| 41 | D | 167 | K |
| 42 | E | 168 | K |
| 43 | Q | 190 | R |
| 44 | P | 191 | R |
| 45 | P | 192 | R |
| 46 | G | 193 | R |
| 47 | I | 194 | R |
| 48 | N | 195 | R |
| 49 | P | 196 | R |
| 50 | S | 197 | R |
| 51 | F | 198 | A |
| 52 | Y | 199 | R |
| 53 | Y | 201 | R |
| 54 | Q | 202 | R |
| 55 | T | 203 | G |
| 56 | I | 204 | R |
| 57 | I | 232 | R |
| 58 | P | 233 | R |
| 59 | L | 234 | R |
| 60 | G | 235 | R |
| 61 | V | 236 | K |
| 62 | V | 237 | K |
| 63 | N | 239 | K |
| 64 | C | 247 | R |
| 65 | P | 248 | R |
| 66 | G | 249 | R |
| 67 | Y | 250 | R |
| 68 | I | 251 | R |
| 69 | P | 252 | R |
| 70 | W | 254 | A |
| 71 | W | 254 | K |
| 72 | Q | 255 | K |
| 73 | A | 261 | R |
| 74 | I | 262 | R |
| 75 | S | 263 | R |
| 76 | P | 264 | R |
| 77 | T | 266 | R |
| 78 | V | 270 | R |
| 79 | T | 271 | R |
| 80 | V | 272 | R |
| 81 | P | 273 | R |
| 82 | G | 274 | R |
| 83 | L | 275 | R |
| 84 | S | 276 | R |
| 85 | P | 277 | R |
| 86 | N | 280 | R |
| 87 | M | 283 | K |
| 88 | Y | 286 | A |
| 89 | Y | 286 | K |
| 90 | W | 287 | A |
| 91 | W | 287 | K |
| 92 | D | 296 | R |
| 93 | A | 297 | R |
| 94 | L | 298 | R |
| 95 | I | 304 | K |
| 96 | D | 312 | K |
| 97 | G | 315 | K |
| 98 | L | 317 | K |
| 99 | N | 319 | K |
| 100 | W | 322 | A |
| 101 | W | 322 | K |
| 102 | F | 339 | A |
| 103 | F | 339 | R |
| 104 | T | 340 | R |
| 105 | G | 341 | R |
| 106 | D | 342 | R |
| 107 | V | 344 | R |
| 108 | D | 346 | K |
| 109 | T | 353 | K |
| 110 | T | 357 | K |
| 111 | Y | 364 | A |
| 112 | W | 368 | A |
| 113 | W | 368 | R |
| 114 | T | 369 | R |
| 115 | G | 372 | R |
| 116 | A | 435 | R |
| 117 | A | 439 | K |
| 118 | W | 440 | R |
| 119 | E | 444 | R |
| 120 | S | 496 | R |
| 121 | N | 497 | R |
| 122 | F | 517 | R |
| 123 | T | 518 | R |
| 124 | P | 519 | R |
| 125 | A | 520 | R |
| 126 | P | 521 | R |
| 127 | V | 533 | K |
| 128 | T | 539 | K |
| 129 | L | 548 | K |
| 130 | Q | 553 | R |
| 131 | L | 560 | R |
| 132 | W | 561 | A |
| 133 | W | 561 | R |
| 134 | E | 569 | R |
| 135 | Y | 570 | A |
| 136 | Y | 570 | K |
| 137 | L | 571 | K |
| 138 | L | 573 | K |
| 139 | S | 574 | K |
| 140 | E | 578 | K |
| 141 | L | 580 | K |
| 142 | N | 586 | K |
| 143 | N | 609 | K |
| 144 | V | 610 | K |
| 145 | F | 612 | A |
| 146 | F | 612 | K |
| 147 | H | 614 | K |
| 148 | G | 615 | R |
| 149 | S | 616 | R |
| 150 | G | 617 | K |
| 151 | T | 628 | R |
| 152 | A | 629 | R |
| 153 | E | 632 | R |
| 154 | Q | 638 | K |
| 155 | T | 649 | R |
| 156 | H | 650 | K |
| 157 | P | 668 | R |
| 158 | C | 670 | R |
| 159 | H | 672 | R |
| 160 | E | 674 | R |
| 161 | E | 681 | R |
| 162 | F | 683 | A |
| 163 | F | 683 | R |
| 164 | Q | 684 | R |
| 165 | G | 689 | R |
| 166 | T | 691 | R |
| 167 | N | 693 | R |
| 168 | T | 700 | R |
| 169 | H | 701 | R |
| 170 | T | 712 | R |

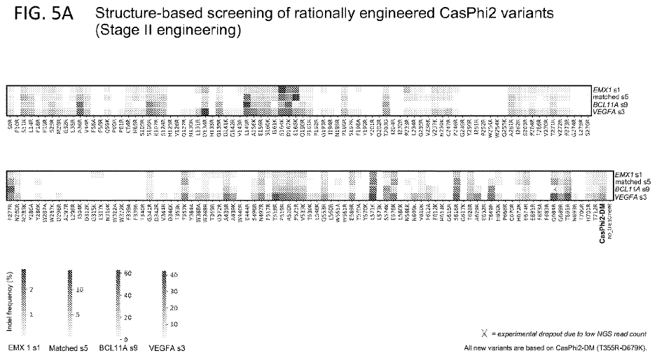

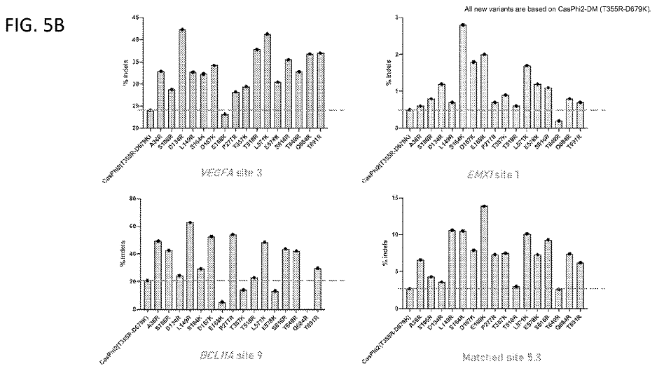

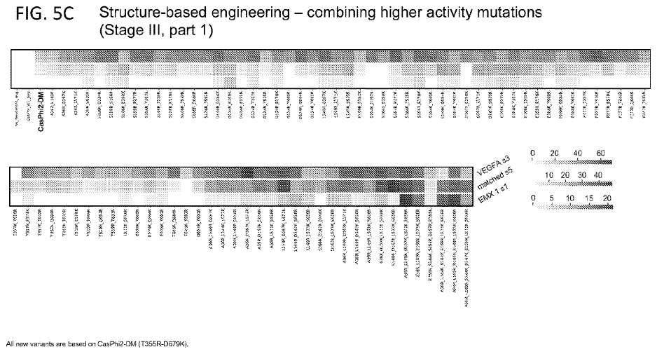

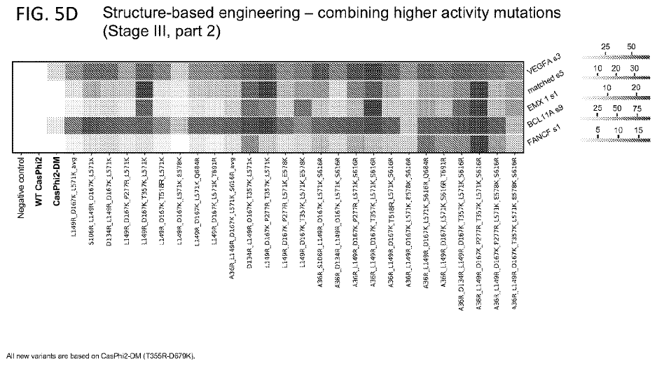

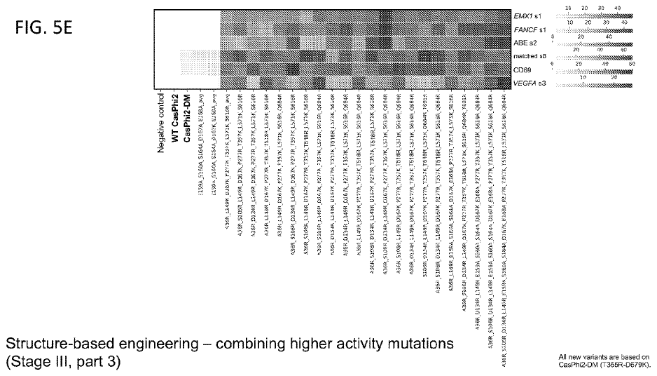

Having identified a total of 159 amino acid positions for potential mutagenesis (156 guided by structure and three based on our analysis of the CasPhi2 nickase variant), we introduced single mutations at each of these positions into the CasPhi2-DM variant and assessed the gene editing activities of the resulting series of triple mutants in human cells. Specifically, we created a total of 170 CasPhi2-DM variants into which we had introduced arginine or lysine substitutions at 148 of the 159 of these positions (choosing one or the other type of substitution depending on the identities of neighboring arginine and/or lysine residues with an eye towards diversifying the types of positively charged residues present in a local region) and arginine, lysine, or alanine substitutions at 11 positions harboring bulky aromatic residues in CasPhi2-DM (Table 10). We then assessed the gene editing activities of each of these 170 variants with four crRNAs targeting different endogenous human gene sites in HEK293T cells (FIG. 5A). The results of this screen yielded 24 candidate variants that appeared to show higher activities than CasPhi2-DM with one or more crRNAs tested (Table 11; note that editing frequencies for a subset of 16 of these 24 variants are shown as bar graphs in FIG. 5B (which regraphs the same data shown in FIG. 5A)).

TABLE 11
Subset of 24 CasPhi2-DM-based variants with one additional
mutation (+X) (in addition to the T355R and D679K
DM mutations) that exhibited increased indel frequencies
with one or more of the four tested crRNAs.

| # | Mutation + X |
|---|---|
| 1 | S11R |
| 2 | A36R |
| 3 | S106R |
| 4 | E107R |
| 5 | S124R |
| 6 | D134R |
| 7 | G138R |
| 8 | L149R |
| 9 | A156K |
| 10 | S160K |
| 11 | S164K |
| 12 | D167K |
| 13 | E168K |
| 14 | T203G |
| 15 | P233R |
| 16 | A261R |
| 17 | P277R |
| 18 | T357K |
| 19 | A435R |
| 20 | N497R |
| 21 | T518R |
| 22 | P519R |
| 23 | A520R |
| 24 | P521R |
| 25 | V533K |
| 26 | E569R |
| 27 | L571K |
| 28 | S574K |
| 29 | E578K |
| 30 | S616R |
| 31 | T628R |
| 32 | T649R |
| 33 | Q684R |
| 34 | T691R |

### Example 5: Engineering Higher Activity CasPhi2 Variants-Stage III (Combinatorial Mutation Testing)

Having identified a set of 24 individual amino acid substitutions that improved the human cell gene editing activity of CasPhi2-DM, we next sought to begin testing various higher order combinations of these mutations to attempt to obtain further efficiency gains. Initially, in Part 1, we created quadruple mutants bearing the DM T355R-D679K mutations together with various pairwise combinations of the 24 substitutions identified from our Stage II experiments and identified a number of variants with even higher gene activities when screened using three different crRNAs in HEK293T cells (FIG. 5C). By testing combinations of variants with increasingly larger numbers of mutations and three or five different crRNAs (Parts 2 and 3), we identified multiple CasPhi2 tetramutants, pentamutants, hexamutants, heptamutants, octamutants, nonamutants, decamutants, undecamutants, and dodecamutants with progressively more efficient human cell gene editing activities (FIGS. 5D and 5E). Additional combinations (including some that also included the E159A, S160A, S164A, and/or E168A mutations from the previously described (in vitro) nickase CasPhi2 variant¹⁶ yielded tridecamutant, tetradecamutant, pentadecamutant, hexadecamutant, and heptadecamutant variants (naming based on IUPAC, wikipedia.org/wiki/IUPAC_numerical_multiplier) that showed more efficient gene editing activities with five different crRNAs in HEK293T cells (FIG. 5E).

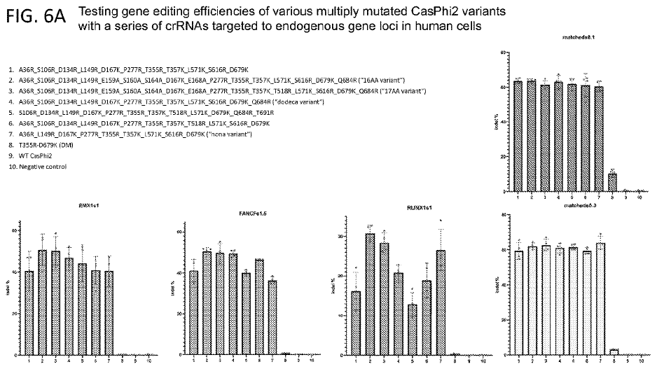

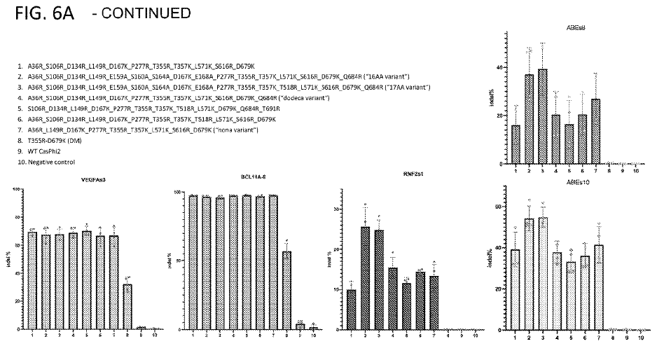

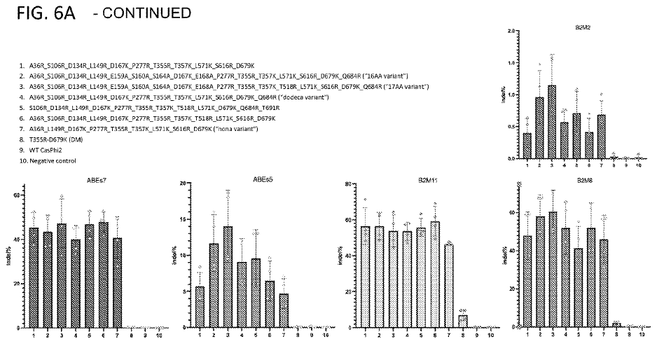

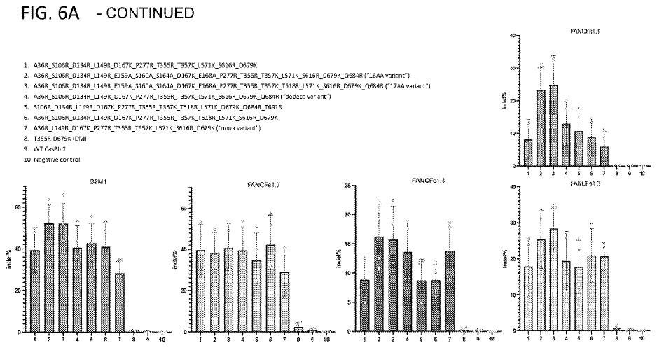

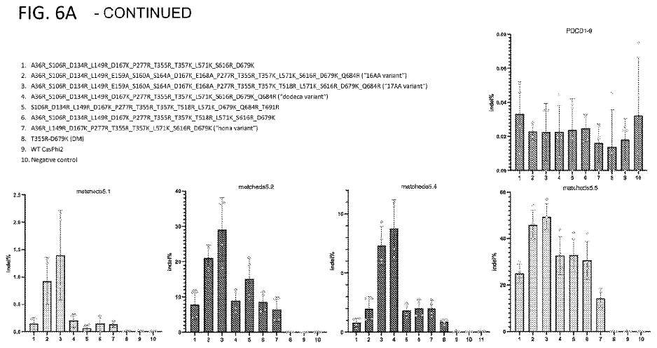

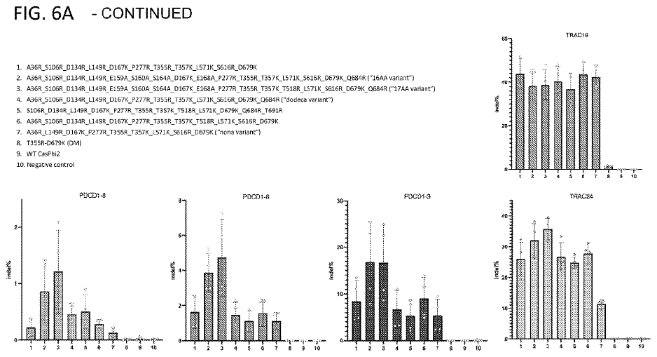

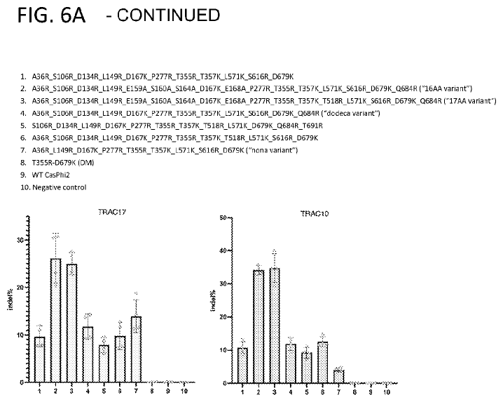

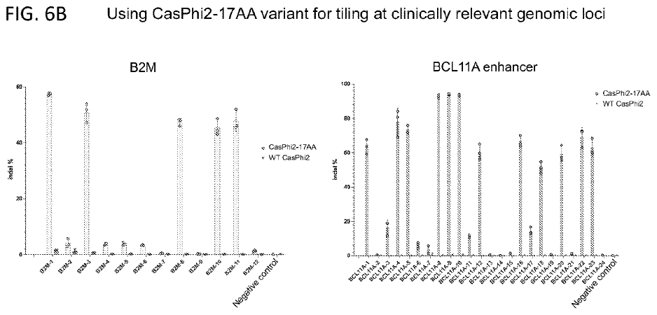

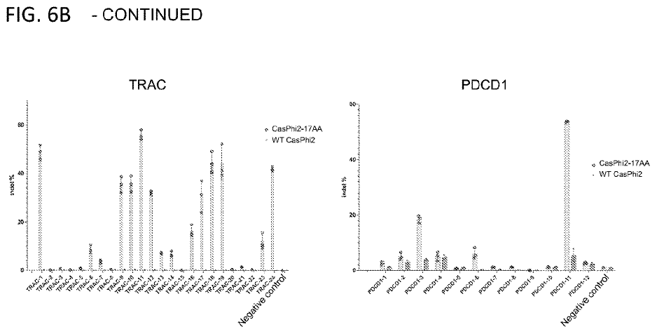

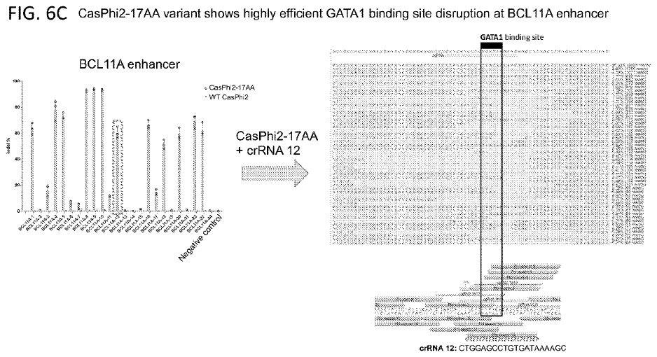

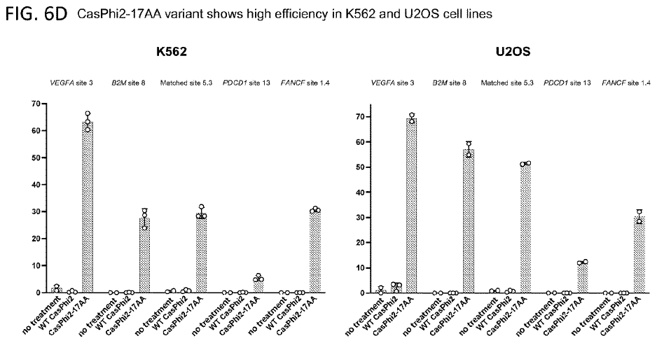

Although many of the multiple substitution CasPhi2 variants we screened showed higher activity in our screens (Table 1), we tested a subset of seven of the most robust and improved enzymes with a larger set of 32 different crRNAs targeting endogenous genes in human cells (FIG. 6A). The seven variants we tested in this experiment included: a nonamutant (A36R/L149R/D167K/P277R/T355R/T357K/L571K/S616R/D679K); a undecamutant (A36R/S106R/D134R/L149R/D167K/P277R/T355R/T357K/L571K/S616R/D679K), three dodecamutants (A36R/S106R/D134R/L149R/D167K/P277R/T355R/T357K/L571K/S616R/D679K/Q68 4R; S106R/D134R/L149R/D167K/P277R/T355R/T357K/T518R/L571K/D679K/Q684R/T69 1R; and A36R/S106R/D134R/L149R/D167K/P277R/T355R/T357K/T518R/L571K/S616R/D679K), a hexadecamutant (A36R/S106R/D134R/L149R/E159A/S160A/S164A/D167K/E168A/P277R/T355R/T357K/L571K/S616R/D679K/Q684R); and a heptadecamutant (A36R/S106R/D134R/L149R/E159A/S160A/S164A/D167K/E168A/P277R/T355R/T357K/T518R/L571K/S616R/D679K/Q684R) (FIG. 6A). All seven of these variants showed consistently and substantially higher gene editing activities relative to both WT CasPhi2 and CasPhi2-DM with 31 of the 32 crRNAs we tested in HEK293T cells (FIG. 6A). (The one crRNA (the PDCD1-9 crRNA) that did not show higher activities with our variants also failed to show evidence of any editing above background with any of the CasPhi2 enzymes we tested (FIG. 6A).) Importantly, for 18 of these 31 crRNAs, at least one of the seven variants showed mean editing frequencies of 20% or more (in many cases with most or all seven variants) and ranging from 20% to >95% (FIG. 6A).

Although we identified many highly active CasPhi2 variants bearing various combinations of nine to 17 mutations, we selected the heptadecamutant (A36R/S106R/D134R/L149R/E159A/S160A/S164A/D167K/E168A/P277R/T355R/T357K/T518R/L571K/S616R/D679K/Q684R, referred to hereafter as CasPhi2-17AA) for more extensive characterization.

We performed side-by-side comparisons of WT CasPhi2 and CasPhi2-17AA by co-transfecting HEK293T cells with plasmids encoding each of these nucleases with plasmids encoding one of 72 different crRNAs targeted to four different clinically relevant genes (12, 24, 24, and 12 crRNAs to the B2M, BCL11A enhancer, TRAC, and PDCD1, respectively) (FIG. 6B). Strikingly, 45 of these 72 crRNAs showed substantially higher editing with CasPhi2-17AA compared with WT CasPhi2 with fold-improvements in editing frequencies ranging from 0.7 to 13,000-fold (FIG. 6B). In addition, the absolute mean frequencies of editing observed with each of these active crRNAs and CasPhi2-17AA were now much higher than what we had observed with CasPhi2-DM (FIG. 6B). With CasPhi2-17AA, four of the B2M crRNAs induced >50% indels, nine of the BCL11A enhancer crRNAs induced >60% indels (with three crRNAs inducing >95% indels), five of the TRAC crRNAs induced >40% indels, and one of the PDCD1 induced >40% indels (FIG. 6B). Notably, the BCL11A-12 crRNA, which disrupts a functionally critical GATA1 binding site in the BCL11A enhancer, yielded ˜60% mean editing frequency with CasPhi2-17AA (FIG. 6C) compared with the much lower <2% editing efficiency observed when we had tested it with CasPhi2-DM (FIG. 2H) and the <1% editing efficiency observed with WT CasPhi2 (FIGS. 6B and 6C). Relative to current SpCas9-based gene editing approaches^{25,26} that can disrupt the GATA1 binding site and that are now being tested in Phase I-III clinical trials (e.g., CLIMB-111, CLIMB-121 and CLIMB-131), CasPhi2-17AA nuclease induces generally longer deletions (FIG. 6C).

To validate that the gains in editing efficiency seen with CasPhi2-17AA in HEK293T could be generalized to other cell types, we tested CasPhi2-17AA in K562 and U2OS cells with 5 crRNAs that had shown varying editing efficiencies in HEK293T cells. Plasmid nucleofection (see Methods section above) of editor and crRNA plasmids yielded editing efficiencies ranging from ˜5-60% in K562 cells and ˜10-70% in U2OS cells (FIG. 6D).

With regard to PAM requirements of the CasPhi2-17AA variant, we note that the most efficient editing was seen at TTN protospacers (which were also targeted predominantly). Of note, we did occasionally see relevant editing at TBN sites, e.g. close to 20% with crRNA PDCD1-3 that targets a site with a TGC-PAM (FIG. 6B).

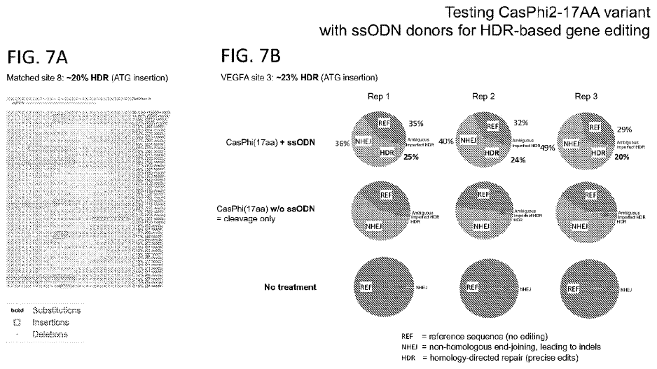

Having characterized the capability of our CasPhi2-17AA variant to induce indel mutations, we also sought to test whether it could stimulate efficient homology-directed repair (HDR) with a donor template. We designed single-stranded oligodeoxynucleotide (ssODN) donors with 40 nt homology arms that were designed to introduce a 3 bp ATG insertion together with PAM-disrupting mutations into target sites in two different endogenous gene loci (matched site 8 and VEGFA site 3) (see Methods section above). We then co-transfected each ssODN with plasmids encoding the cognate crRNA and CasPhi2-17AA into HEK293T cells and used targeted amplicon sequencing to assess mutations at the on-target sites. These experiments showed that CasPhi2-17AA could induce desired HDR edits with frequencies of ˜20% with the matched site 8 crRNA (FIG. 7A) and of ˜20 to 25% with the VEGFA site 3 crRNA (FIG. 7B). As expected, we also observed indels at both target sites (FIGS. 7A and 7B), presumably generated by NHEJ/MMEJ-mediated DNA repair of the nuclease-induced DNA break. Taken together, our experiments demonstrate that CasPhi2-17AA can induce both indels and HDR-mediated alterations with high efficiencies in human cells.

### Example 7: Engineering and Characterization of CasPhi2-17AA-Based Fusion Proteins for Base Editing Activities

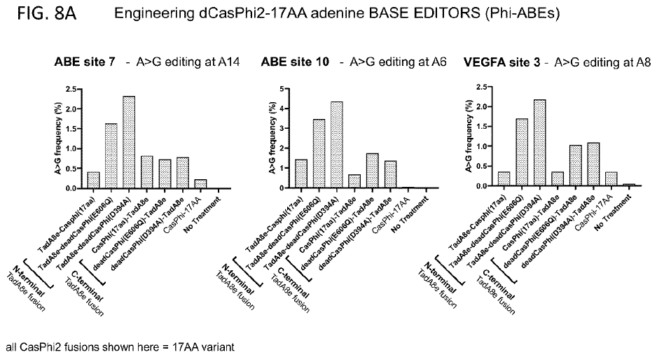

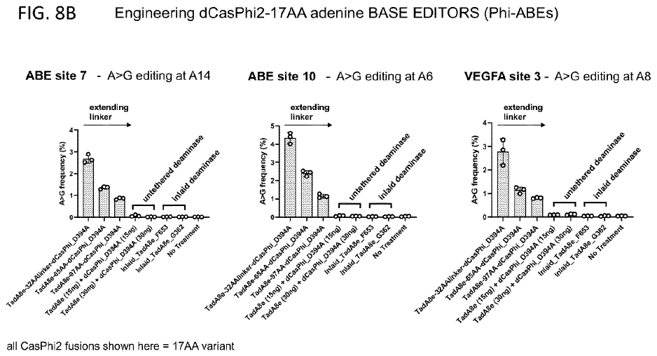

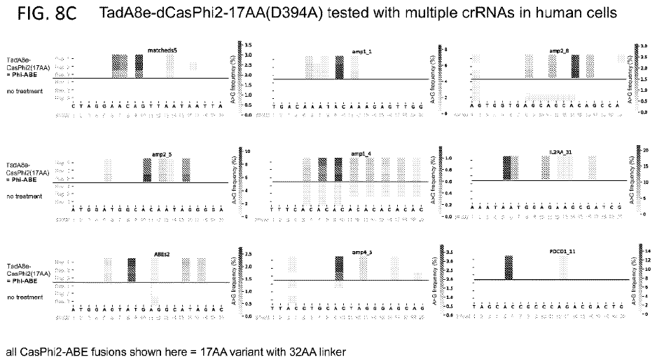

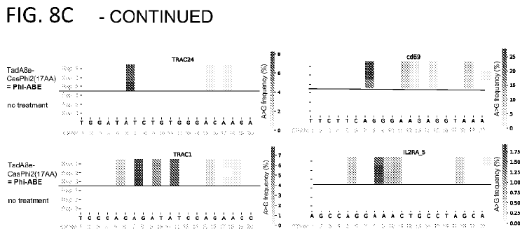

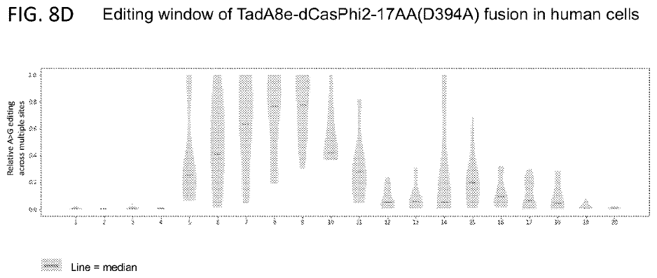

Having established the nuclease-based gene editing activities of CasPhi2-17AA, we next sought to determine whether a catalytically inactive or catalytically impaired mutant of this variant (dCasPhi2-17AA (D394A) or dCasPhi2-17AA (E606Q), respectively) might function to mediate targeted base editing. Because we did not observe any adenine base editing in our earlier attempts with dCasPhi2-DM (FIG. 2I above), we constructed a variety of different dCasPhi2-17AA-based adenine base editor architectures. Specifically, we constructed expression plasmids encoding fusions of the TadA8e adenine deaminase²⁴ fused to the N- or C-terminus of CasPhi2-17AA, catalytically inactive dCasPhi2-17AA (D394A), or catalytically impaired dCasPhi2-17AA (E606Q) with a 32AA modified XTEN linker (flanked with extended GlySer linkers on both sides; see Table 5 above)²⁷⁻²⁹. We then co-transfected HEK293T cells in triplicate with combinations of each of these plasmids and each of three different crRNAs targeting various human genomic loci (ABE site 7, ABE site 10, VEGFA site 3) and then performed targeted amplicon sequencing of the target sites to assess the frequencies of adenine base editing (see Methods section above). The results of these experiments demonstrated measurable adenine editing with all six fusion proteins with at least one of the crRNAs with mean frequencies as high as ˜4% (FIG. 8A). Overall, these experiments also showed that N-terminal TadA8e fusions were more efficient than corresponding C-terminal fusions and that editing rates were highest with fusions harboring catalytically inactive dCasPhi2-17AA (D394A) (FIG. 8A). Interestingly, the use of longer 65 AA or 97 AA linkers (multiples of the original 32 AA linker; see Table 5 above) in the N-terminal dCasPhi2-17AA (D394A) fusions led to progressively less efficient base editing (FIG. 8B). In addition, testing two inlaid fusions of the TadA8e deaminase within dCasPhi2-17AA (D394A) (inserted just carboxy-terminal to amino acid positions G362 and F653) and expression of separate, untethered TadA8e deaminase and dCasPhi2-17AA (D394A) did not induce detectable adenine base editing (FIG. 8B). Taken together, these observations suggest that the base editing activity we observe with these fusions is dependent on tethering of the deaminase domain to the dCasPhi2-17AA protein.

We performed more extensive characterization of protein in which TadA8e deaminase is fused to the N-terminus of dCasPhi2-17AA (D394A) protein (hereafter referred to as TadA8e-dCasPhi2-17AA (D394A)) by testing it with 13 additional crRNAs targeted to various endogenous genomic loci in human cells. We co-transfected plasmid encoding dCasPhi2-17AA (D394A) with plasmid expressing each of the 13 different crRNAs in triplicate into HEK293T cells and then assessed adenine base editing at the on-target sites using targeted amplicon sequencing (see Methods section above). This experiment revealed A>G editing frequencies ranging from <1% to >25% across the different target sites tested (FIG. 8C). Analysis of the locations of editing events within the target spacers defined a PAM-proximal editing window covering positions 5 to 11 (numbered relative to the PAM) with highest editing efficiencies at positions 7-9 (FIG. 8D). In addition, we also observed a second, weaker editing window centered at spacer position 15 (FIG. 8D).

Overall, we conclude from these experiments that the CasPhi2-17AA variant provides an RNA-guided protein that can be used to induce efficient adenine base editing in human cells.

### Example 8: Engineering and Characterization of CasPhi2-17AA-Based Fusion Proteins for Epigenetic Editing Activities

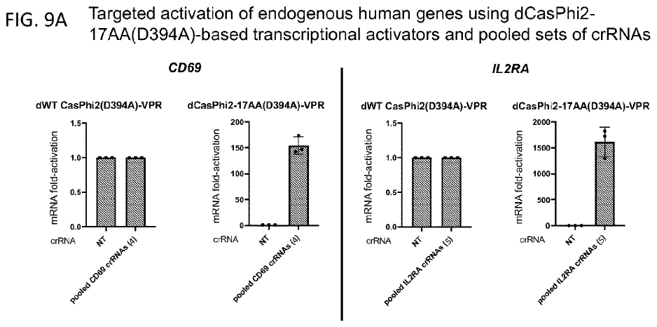

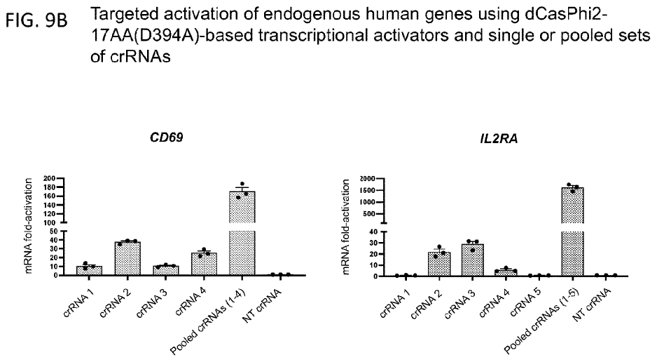

We also tested whether dCasPhi2-17AA (D394A) might be used to create targetable epigenetic editors that function efficiently in human cells. To do this, we constructed an expression plasmid that expresses a fusion of the VPR activation domain to the C-terminus of dCasPhi2-17AA (D394A), similar to our initial attempt to make CasPhi2-DM based activators (FIG. 2J above). We then performed co-transfections of plasmid expressing dCasPhi2-17AA (D394A)-VPR fusion or dWT CasPhi2 (D394A)-VPR fusion with a pool of plasmids expressing different crRNAs targeting the either the (D) 69 (four crRNAs) or IL2RA (five crRNAs) gene promoters and then measured fold-activation of the target gene by quantitative real-time PCR (see Methods section above). The dCasPhi2-17AA(D394)-VPR fusion robustly activated both target genes: ˜150-fold for CD69 and ˜1500-fold for IL2RA (FIG. 9A). By contrast, dWTCasPhi2(D394A)-VPR fusion failed to activate both target genes (FIG. 9A). We additionally tested how well each of individual crRNAs we had used together in pooled format would function to activate the CD69 and IL2RA promoters in HEK293T cells with dCasPhi2-17AA(D394A)-VPR. For CD69, all four of the individual crRNAs could activate the promoter ˜10-fold to ˜35-fold with dCasPhi2-17AA(D394)-VPR (FIG. 9B). For IL2RA, three of the five individual crRNAs activated the promoter ˜5-fold to ˜30-fold with dCasPhi2-17AA(D394)-VPR. Based on these results, we conclude that dCasPhi2-17AA(D394A) can be used to create VPR activator fusions that can function robustly with either single or multiple crRNAs to mediate targeted transcriptional activation of endogenous human genes, suggesting that this CasPhi2 variant should also work for other types of epigenetic editing (e.g., by fusing histone modifying enzymes, DNA methylases, TET1 catalytic domain, and other domains expected to influence gene regulation)³⁰.

### Example 9: Screening of Additional Mutations in CasPhi2 that Increase its Gene Editing Nuclease Activity in Human Cells

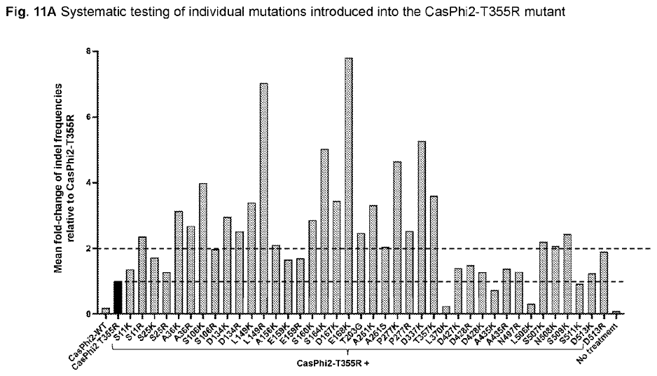

Given our success in identifying single amino acid changes that improve the activity of CasPhi2 in human cells, we screened a larger set of such mutations to find more activity-enhancing alterations. To do this, we added a series of 82 different single amino acid substitutions (Table 12) to a CasPhi2 mutant bearing a T335R mutation (which had shown higher activity in human cells relative to wild-type CasPhi2-see above). The 82 mutations included new types of amino acid substitutions at positions we had previously identified as well as at additional residues that lie within a lysine-rich loop (spanning amino acids V510-R535), α-helices 17 and 18 (residues S469-K545), and a loop near the enzyme active site (including residue R716). We tested each of these various 82 variants for their abilities to induce gene editing at six different endogenous gene target sites in human HEK293T cells (as assessed by targeted amplicon sequencing—see, Methods section above) and calculated the mean fold-change in indel frequencies relative to CasPhi2-T335R across all six target sites tested (FIGS. 11A-11B). The results of this analysis identified 43 different amino acid substitutions that showed a two-fold or greater mean fold-change in editing activity relative to CasPhi2-T335R across the six different target sites (Table 12). Indeed, several of these variants showed substantially higher mean fold-changes of four- to nearly eight-fold (FIGS. 11A-11B).

TABLE 12
CasPhi2 T355R variants with one additional mutation (+X).

| # | CasPhi2 T355R + X |
|---|---|
| 1 | S11K |
| 2 | S11R |
| 3 | S25K |
| 4 | S25R |
| 5 | A36K |
| 6 | A36R |
| 7 | S106K |
| 8 | S106R |
| 9 | D134K |
| 10 | D134R |
| 11 | L149K |
| 12 | L149R |
| 13 | A156K |
| 14 | E159K |
| 15 | E159R |
| 16 | S160K |
| 17 | S164K |
| 18 | D167K |
| 19 | E168K |
| 20 | T203G |
| 21 | A261K |
| 22 | A261S |
| 23 | P277K |
| 24 | P277R |
| 25 | D337K |
| 26 | T357K |
| 27 | L370K |
| 28 | D427K |
| 29 | D428R |
| 30 | D428K |
| 31 | A435K |
| 32 | A435R |
| 33 | N497R |
| 34 | L506K |
| 35 | S507K |
| 36 | N508K |
| 37 | S509K |
| 38 | S511K |
| 39 | D513K |
| 40 | D513R |
| 41 | Q514K |
| 42 | T518K |
| 43 | T518R |
| 44 | P519R |
| 45 | A520K |
| 46 | A520R |
| 47 | G524K |
| 48 | A525K |
| 49 | K526G |
| 50 | K527G |
| 51 | P530K |
| 52 | P530R |
| 53 | V531K |
| 54 | V531R |
| 55 | E532K |
| 56 | E532R |
| 57 | V533K |
| 58 | R538A |
| 59 | R538S |
| 60 | R538G |
| 61 | T539A |
| 62 | T539K |
| 63 | A543R |
| 64 | A543K |
| 65 | E569K |
| 66 | L571K |
| 67 | E578K |
| 68 | S616K |
| 69 | S616R |
| 70 | T628R |
| 72 | T649K |
| 73 | E674R |
| 74 | E674K |
| 75 | E674S |
| 76 | E674G |
| 77 | G676K |
| 78 | D679K |
| 80 | Q684K |
| 81 | Q684R |
| 82 | T691K |

TABLE 13
CasPhi2 T355R-based variants with one additional mutation (+X)
that exhibited a two-fold or greater mean fold-change in editing
activity relative to CasPhi2-T335R across six different target sites

| # | CasPhi2 T355R + X |
|---|---|
| 1 | S11R |
| 2 | A36K |
| 3 | A36R |
| 4 | S106K |
| 5 | D134K |
| 6 | D134R |
| 7 | L149K |
| 8 | L149R |
| 9 | A156K |
| 10 | S160K |
| 11 | S164K |
| 12 | D167K |
| 13 | E168K |
| 14 | T203G |
| 15 | A261K |
| 16 | A261S |
| 17 | P277K |
| 18 | P277R |
| 19 | D337K |
| 20 | T357K |
| 21 | S507K |
| 22 | N508K |
| 23 | S509K |
| 24 | A520K |
| 25 | A520R |
| 26 | A525K |
| 27 | P530R |
| 28 | V531K |
| 29 | V531R |
| 30 | E532K |
| 31 | E532R |
| 32 | R538G |
| 33 | T539A |
| 34 | A543R |
| 35 | A543K |
| 36 | E569K |
| 37 | L571K |
| 38 | E578K |
| 39 | S616K |
| 40 | S616R |
| 41 | E674S |
| 42 | G676K |
| 43 | D679K |

### Example 10: Engineering Additional Highly Active CasPhi2 Variants Lacking Mutations within α-Helix 7

Previous work has suggested that α-helix 7 (residues V143 to N195 as defined and claimed in patent application WO 2022/159822 A1) of the CasPhi2 RecI domain plays an important role in catalytic activity by modulating substrate accessibility to the RuvC active site domain¹⁶. Six of the 17 different mutations we introduced to engineer the highly active CasPhi2-17AA variant described above lie within α-helix 7 (L149, E159, S160, S164, D167, E168). We were interested in exploring whether mutations within α-helix 7 are required to generate CasPhi2 with high activities in human cells or whether such variants could be generated without alterations within this alpha-helix. To begin this work, we generated two variants:
1) A CasPhi2-11AA variant that harbors 11 of the 17 mutations present in the CasPhi2-17AA variant (Table 14). These 11 mutations all fall outside the α-helix 7 region.
2) A CasPhi2-11(+1)AA harboring the same 11 mutations present in the CasPhi2-11AA variant and one additional mutation (L149R) within α-helix 7 (Table 14).

TABLE 14
Mutations present in the CasPhi2-17AA, CasPhi2-11AA, and
CasPhi2-11 + 1AA variants (α-helix 7 mutations are underlined).

| | |
|---|---|
| CasPhi2-17AA | A36R, S106R, D134R, L149R, E159A, S160A, S164A, D167K, E168A, |
| | P277R, T355R, T357K, T518R, L571K, S616R, D679K, Q684R |
| CasPhi2-11AA | A36R, S106R, D134R, P277R, T355R, T357K, T518R, L571K, S616R, |
| | D679K, Q684R |
| CasPhi2-11 + 1AA | A36R, S106R, D134R, L149R, P277R, T355R, T357K, T518R, L571K, |
| | S616R, D679K, Q684R |

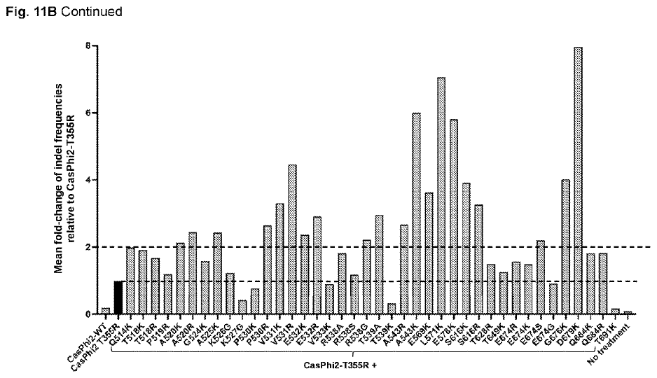

We compared the gene editing activities of these two new CasPhi2 variants with that of the CasPhi2-17AA variant by co-expressing each of these variants with one of 16 different crRNAs targeting various genomic endogenous gene sites in HEK293T cells and assessing on-target indel frequencies using targeted amplicon sequencing (Methods). These experiments demonstrated the CasPhi2-11AA and CasPhi2-11+1AA variants, like the CasPhi2-17AA variant, showed robust gene editing activities across the 16 different target sites (FIG. 12). Indeed, the CasPhi2-11AA and CasPhi2-11+1AA variants showed gene editing efficiencies that were ˜50% or more of that observed with the CasPhi2-17AA variant for 10 of the 16 sites and for 14 of the 16 sites, respectively (FIG. 12). Furthermore, although the presence of the additional L149R mutation in CasPhi2-11+1AA appeared to generally increase activity relative to the CasPhi2-11AA variant, this increase was relatively modest in many cases (FIG. 12). Thus, we conclude that mutations in alpha-helix 7 are not required to generate high activity CasPhi2 variants and mutations in other parts of the protein contribute substantially to the high activity of our CasPhi2-17AA variant.

### Example 11: Engineering of High Activity CasPhi2 Variants Devoid of Amino Acid Substitutions within α-Helix 7

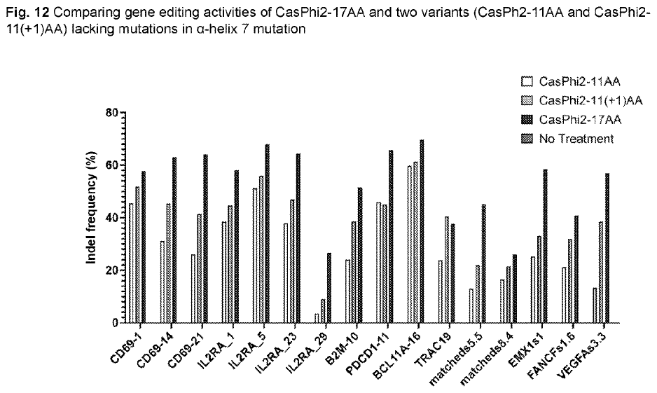

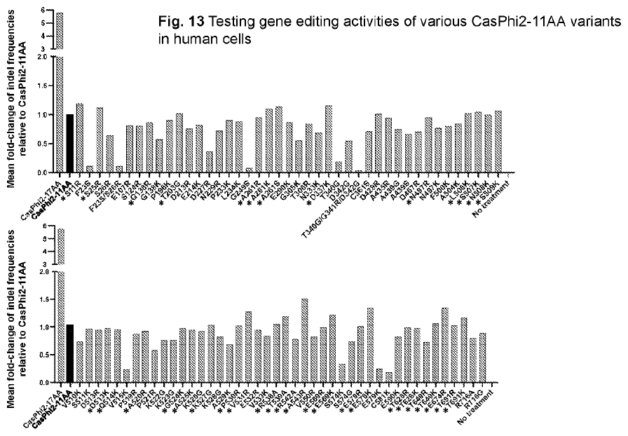

Encouraged by the robust gene editing activity of the CasPhi2-11AA variant, we explored whether we might be able to increase its activity by adding additional amino acid substitutions that lie outside of α-helix 7. In an initial screen, we created a series of 87 different derivatives of CasPhi2-11AA (Table 15) that harbored an additional single amino acid substitution (85 different variants), a double amino acid substitution (F23S/S26R), or a triple amino acid substitution (T340G/D341R/D342G). These mutations all lie outside of α-helix 7 and had all shown an ability to increase the human cell-based gene editing activity of CasPhi2 or CasPhi2 variants as described in detail above. We assessed the gene editing activities of these 87 variants and the parental CasPhi2-11AA variant with crRNAs targeting eight different endogenous gene sites (B2M site 2, FANCF site 1.6, PDCD1 site 6, matched site 5.2, VEGFA site 3, BCL11A site 9, matched site 5.3, EMX1 site 1) in HEK293T cells with indel frequencies quantified using targeted amplicon sequencing (FIG. 13). This experiment identified 36 single amino acid substitutions that increased the gene editing activities (on-target indel frequencies) of CasPhi2-11A with at least two of the eight crRNAs tested (FIG. 13 and Table 16).

TABLE 15
List of mutations introduced into the CasPhi2-11AA
variant and screened for increased gene editing activities
in human cells with 8 different crRNAs.

| # | |
|---|---|
| 1 | S11R |
| 2 | F23S |
| 3 | S25R |
| 4 | S26R |
| 5 | E107R |
| 6 | S124R |
| 7 | G138R |
| 8 | G138K |
| 9 | P196K |
| 10 | T203G |
| 11 | D213R |
| 12 | E214K |
| 13 | D227R |
| 14 | N229R |
| 15 | P233K |
| 16 | L234K |
| 17 | G249S |
| 18 | A261K |
| 19 | A261R |
| 20 | A261S |
| 21 | E290K |
| 22 | G305K |
| 23 | T306R |
| 24 | N333K |
| 25 | D337K |
| 26 | T340G |
| 27 | D342G |
| 28 | C361S |
| 29 | D428R |
| 30 | A435R |
| 31 | A439G |
| 32 | A439S |
| 33 | D467R |
| 34 | N497R |
| 35 | N497K |
| 36 | F500K |
| 37 | A504K |
| 38 | L506K |
| 39 | S507K |
| 40 | N508K |
| 41 | S509K |
| 42 | V510K |
| 43 | S511K |
| 44 | D513K |
| 45 | D513R |
| 46 | Q514K |
| 47 | V515K |
| 48 | P519R |
| 49 | A520R |
| 50 | P521R |
| 51 | K522G |
| 52 | K523G |
| 53 | G524K |
| 54 | A525K |
| 55 | K526G |
| 56 | K527G |
| 57 | K528G |
| 58 | A529K |
| 59 | P530R |
| 60 | V531R |
| 61 | E532R |
| 62 | V533K |
| 63 | R538A |
| 64 | T539A |
| 65 | R542A |
| 66 | A543R |
| 67 | V550R |
| 68 | E569R |
| 69 | E569K |
| 70 | S574K |
| 71 | S574G |
| 72 | E578R |
| 73 | E578K |
| 74 | E579K |
| 75 | C581K |
| 76 | E590K |
| 77 | T628R |
| 78 | T628K |
| 79 | T649R |
| 80 | T649K |
| 81 | E674R |
| 82 | T691R |
| 83 | T691K |
| 84 | R716A |
| 85 | R716G |
| 86 | F23S_S26R |
| 87 | T340G_D341R_D342G |

TABLE 16
List of 36 variants derived from CasPhi2-11A harboring one additional
mutation (+X) that exhibited higher gene editing activities
in human cells with two or more of the eight crRNAs tested.

| # | CasPhi2-11AA + X |
|---|---|
| 1 | S11R |
| 2 | S25R |
| 3 | G138R |
| 4 | T203G |
| 5 | A261R |
| 6 | A261K |
| 7 | A261S |
| 8 | D337K |
| 9 | N497R |
| 10 | L506K |
| 11 | S507K |
| 12 | N508K |
| 13 | S509K |
| 14 | D513K |
| 15 | Q514K |
| 16 | A520R |
| 17 | G524K |
| 18 | A525K |
| 19 | K527G |
| 20 | P530R |
| 21 | V531R |
| 22 | R538A |
| 23 | T539A |
| 24 | R542A |
| 25 | A543R |
| 26 | E569R |
| 27 | E569K |
| 28 | E578R |
| 29 | E578K |
| 30 | T628R |
| 31 | T628K |
| 32 | T649R |
| 33 | T649K |
| 34 | E674R |
| 35 | T691R |
| 36 | T691K |

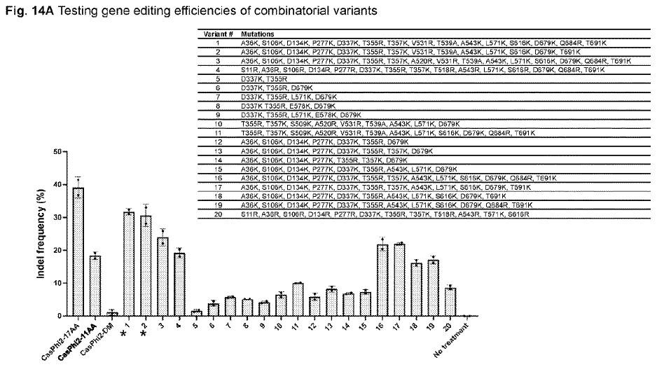

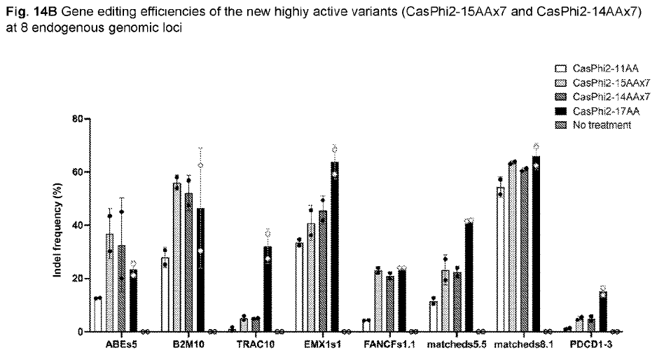

We next created a series of 20 different CasPhi2 variants bearing various combinations of amino acid substitutions we identified in our various analyses described above but specifically lacking any mutations within α-helix 7 (Table 17). We tested the gene editing activities of these 20 CasPhi2 variants with crRNAs targeting eight different endogenous genomic loci in HEK293T cells, directly comparing mean indel frequencies induced by these 20 variants across these eight sites with those of the CasPhi2-DM, CasPhi2-11AA, and CasPhi2-17AA variants we had previously generated (FIG. 14A). This experiment yielded two new variants #1 and #2 that induced mean indel frequencies of 32% and 31%, respectively across the eight different target sites, frequencies higher than that of CasPhi2-11AA (mean indel frequency of 26%) and only slightly lower than that of CasPhi2-17AA (mean indel frequency of 39%) (FIG. 14A and Table 17). We named these two variants (#1 and #2), which harbor 15 and 14 amino acid substitutions, CasPhi2-15AAx7 and CasPhi2-14AAx7, respectively, with ×7 indicating the absence of any amino acid substitutions within α-helix 7 (Table 18). Interestingly, closer examination of the mean indel frequencies induced at the eight individual target sites revealed that CasPhi2-15AAx7 and CasPhi2-14AAx7 exhibited comparable or higher gene editing activities than CasPhi2-17AA at four of the eight sites and ˜50% or more of the activity of CasPhi2-17AA at two of the four sites (FIG. 14B). At the remaining two sites, CasPhi2-15AAx7 and CasPhi2-14AAx7 both exhibited higher gene editing activities than the CasPhi2-11AA variant (FIG. 14B). Taken together, our results clearly demonstrate the feasibility of creating CasPhi2 variants with high gene editing activities in human cells that do not contain any amino acid substitutions within α-helix 7.

TABLE 17
20 additional CasPhi2 variants tested with 8 different crRNAs in HEK293T cells.

| Variant # | Mutations |
|---|---|
| 1 | A36K, S106K, D134K, P277K, D337K, T355R, T357K, V531R, T539A, |
| (CasPhi2- | A543K, L571K, S616K, D679K, Q684R, T691K |
| 15AAx7) | |
| 2 | A36K, S106K, D134K, P277K, D337K, T355R, T357K, V531R, T539A, |
| (CasPhi2- | A543K, L571K, S616K, D679K, T691K |
| 14AAx7) | |
| 3 | A36K, S106K, D134K, P277K, D337K, T355R, T357K, A520R, V531R, |
| | T539A, A543K, L571K, S616K, D679K, Q684R, T691K |
| 4 | S11R, A36R, S106R, D134R, P277R, D337K, T355R, T357K, T518R, |
| | A543R, L571K, S616R, D679K, Q684R, T691K |
| 5 | D337K, T355R |
| 6 | D337K, T355R, D679K |
| 7 | D337K, T355R, L571K, D679K |
| 8 | D337K T355R, E578K, D679K |
| 9 | D337K, T355R, L571K, E578K, D679K |
| 10 | T355R, T357K, S509K, A520R, V531R, T539A, A543K, L571K, D679K |
| 11 | T355R, T357K, S509K, A520R, V531R, T539A, A543K, L571K, S616K, |
| | D679K, Q684R, T691K |
| 12 | A36K, S106K, D134K, P277K, D337K, T355R, D679K |
| 13 | A36K, S106K, D134K, P277K, D337K, T355R, T357K, D679K |
| 14 | A36K, S106K, D134K, P277K, T355R, T357K, D679K |
| 15 | A36K, S106K, D134K, P277K, D337K, T355R, A543K, L571K, D679K |
| 16 | A36K, S106K, D134K, P277K, D337K, T355R, T357K, A543K, L571K, |
| | S616K, D679K, Q684R, T691K |
| 17 | A36K, S106K, D134K, P277K, D337K, T355R, T357K, A543K, L571K, |
| | S616K, D679K, T691K |
| 18 | A36K, S106K, D134K, P277K, D337K, T355R, A543K, L571K, S616K, |
| | D679K, T691K |
| 19 | A36K, S106K, D134K, P277K, D337K, T355R, A543K, L571K, S616K, |
| | D679K, Q684R, T691K |
| 20 | S11R, A36R, S106R, D134R, P277R, D337K, T355R, T357K, T518R, |
| | A543R, T571K, S616R |

TABLE 18
Detailed comparisons of amino acid substitutions present in the
high activity CasPhi2-17AA, CasPhi2-11A, CasPhi2-15AAx7, and
CasPhi2-14AAx7 variants. Amino acid substitutions at positions
that lie within α-helix 7 are indicated with an asterisk.

| Residue | CasPhi2- | CasPhi2- | #1 = CasPhi2- | #2 = CasPhi2- |
|---|---|---|---|---|
| changes | 17AA | 11AA | 15AAx7 | 14AAx7 |
| A36R | A36R | A36R | A36K | A36K |
| S106R | S106R | S106R | S106K | S106K |
| D134R | D134R | D134R | D134K | D134K |
| L149R* | L149R* | | | |
| E159A* | E159A* | | | |
| S160A* | S160A* | | | |
| S164A* | S164A* | | | |
| D167K* | D167K* | | | |
| E168A* | E168A* | | | |
| P277R | P277R | P277R | P277K | P277K |
| D337K | | | D337K | D337K |
| T355R | T355R | T355R | T355R | T355R |
| T357K | T357K | T357K | T357K | T357K |
| T518R | T518R | T518R | | |
| V531R | | | V531R | V531R |
| T539A | | | T539A | T539A |
| A543K | | | A543K | A543K |
| L571K | L571K | L571K | L571K | L571K |
| S616R | S616R | S616R | S616K | S616K |
| D679K | D679K | D679K | D679K | D679K |
| Q684R | Q684R | Q684R | Q684R | |
| T691K | | | T691K | T691K |

### Sequences:

WT CasPhi2 with dual bpNLS fused to N- and C-termini (pJUL2552)
(SEQ ID NO: 15)
MKRTADGSEFESPKKKRKVPKPAVESEFSKVLKKHFPGERFRSSYMKRGGKILAA
QGEEAVVAYLQGKSEEEPPNFQPPAKCHVVTKSRDFAEWPIMKASEAIQRYIYAL
STTERAACKPGKSSESHAAWFAATGVSNHGYSHVQGLNLIFDHTLGRYDGVLKKV
QLRNEKARARLESINASRADEGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSPKKNKRMRRYWRSEKEKAQDALLVTVRIGTDWVVID
VRGLLRNARWRTIAPKDISLNALLDLFTGDPVIDVRRNIVTFTYTLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFTPAPKKGAKKKAPVE
VMRKDRTWARAYKPRLSVEAQKLKNEALWALKRTSPEYLKLSRRKEELCRRSINY
VIEKTRRRTQCQIVIPVIEDLNVRFFHGSGKRLPGWDNFFTAKKENRWFIQGLHK
AFSDLRTHRSFYVFEVRPERTSITCPKCGHCEVGNRDGEAFQCLSCGKTCNADLD
VATHNLTQVALTGKTMPKREEPRDAQGTAPARKTKKASKSKAPPAEREDQTPAQE
PSQTSGGSKRTADGSEFEPKKKRKV
CasPhi2-DM (T355R-D679K) (pBM3491)
(SEQ ID NO: 16)
MKRTADGSEFESPKKKRKVPKPAVESEFSKVLKKHFPGERFRSSYMKRGGKILAA
QGEEAVVAYLQGKSEEEPPNFQPPAKCHVVTKSRDFAEWPIMKASEAIQRYIYAL
STTERAACKPGKSSESHAAWFAATGVSNHGYSHVQGLNLIFDHTLGRYDGVLKKV
QLRNEKARARLESINASRADEGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSPKKNKRMRRYWRSEKEKAQDALLVTVRIGTDWVVID
VRGLLRNARWRTIAPKDISLNALLDLFTGDPVIDVRRNIVTFRYTLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFTPAPKKGAKKKAPVE
VMRKDRTWARAYKPRLSVEAQKLKNEALWALKRTSPEYLKLSRRKEELCRRSINY
VIEKTRRRTQCQIVIPVIEDLNVRFFHGSGKRLPGWDNFFTAKKENRWFIQGLHK
AFSDLRTHRSFYVFEVRPERTSITCPKCGHCEVGNRKGEAFQCLSCGKTCNADLD
VATHNLTQVALTGKTMPKREEPRDAQGTAPARKTKKASKSKAPPAEREDQTPAQE
PSQTSGGSKRTADGSEFEPKKKRKV
CasPhi2-PENTA (L149R-D167K-T355R-L571K-D679K) with dual bpNLS (pEH1316)
MKRTADGSEFESPKKKRKVPKPAVESEFSKVLKKHFPGERFRSSYMKRGGKILA
(SEQ ID NO: 17)
AQGEEAVVAYLQGKSEEEPPNFQPPAKCHVVTKSRDFAEWPIMKASEAIQRYIYA
LSTTERAACKPGKSSESHAAWFAATGVSNHGYSHVQGLNLIFDHTLGRYDGVLKK
VQRRNEKARARLESINASRAKEGLPEIKAEEEEVATNETGHLLQPPGINPSFYVY
QTISPQAYRPRDEIVLPPEYAGYVRDPNAPIPLGVVRNRCDIQKGCPGYIPEWQR
EAGTAISPKTGKAVTVPGLSPKKNKRMRRYWRSEKEKAQDALLVTVRIGTDWVVI
DVRGLLRNARWRTIAPKDISLNALLDLFTGDPVIDVRRNIVTFRYTLDACGTYAR
KWTLKGKQTKATLDKLTATQTVALVAIDLGQTNPISAGISRVTQENGALQCEPLD
RFTLPDDLLKDISAYRIAWDRNEEELRARSVEALPEAQQAEVRALDGVSKETART
QLCADFGLDPKRLPWDKMSSNTTFISEALLSNSVSRDQVFFTPAPKKGAKKKAPV
EVMRKDRTWARAYKPRLSVEAQKLKNEALWALKRTSPEYKKLSRRKEELCRRSIN
YVIEKTRRRTQCQIVIPVIEDLNVRFFHGSGKRLPGWDNFFTAKKENRWFIQGLH
KAFSDLRTHRSFYVFEVRPERTSITCPKCGHCEVGNRKGEAFQCLSCGKTCNADL
DVATHNLTQVALTGKTMPKREEPRDAQGTAPARKTKKASKSKAPPAEREDQTPAQ
EPSQTSGGSKRTADGSEFEPKKKRKV
CasPhi2-HEXA (L149R-D167K-T355R-T357K-L571K-D679K), dual bpNLS
(pEH1476)
(SEQ ID NO: 18)
MKRTADGSEFESPKKKRKVPKPAVESEFSKVLKKHFPGERFRSSYMKRGGKILAA
QGEEAVVAYLQGKSEEEPPNFQPPAKCHVVTKSRDFAEWPIMKASEAIQRYIYAL
STTERAACKPGKSSESHAAWFAATGVSNHGYSHVQGLNLIFDHTLGRYDGVLKKV
QRRNEKARARLESINASRAKEGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSPKKNKRMRRYWRSEKEKAQDALLVTVRIGTDWVVID
VRGLLRNARWRTIAPKDISLNALLDLFTGDPVIDVRRNIVTFRYKLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFTPAPKKGAKKKAPVE
VMRKDRTWARAYKPRLSVEAQKLKNEALWALKRTSPEYKKLSRRKEELCRRSINY
VIEKTRRRTQCQIVIPVIEDLNVRFFHGSGKRLPGWDNFFTAKKENRWFIQGLHK
AFSDLRTHRSFYVFEVRPERTSITCPKCGHCEVGNRKGEAFQCLSCGKTCNADLD
VATHNLTQVALTGKTMPKREEPRDAQGTAPARKTKKASKSKAPPAEREDQTPAQE
PSQTSGGSKRTADGSEFEPKKKRKV
CasPhi2-HEPTA1 (A36R-L149R-D167K-T355R-L571K-S616R-D679K), dual bpNLS
(pEH1328)
(SEQ ID NO: 19)
MKRTADGSEFESPKKKRKVPKPAVESEFSKVLKKHFPGERFRSSYMKRGGKILRA
QGEEAVVAYLQGKSEEEPPNFQPPAKCHVVTKSRDFAEWPIMKASEAIQRYIYAL
STTERAACKPGKSSESHAAWFAATGVSNHGYSHVQGLNLIFDHTLGRYDGVLKKV
QRRNEKARARLESINASRAKEGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSPKKNKRMRRYWRSEKEKAQDALLVTVRIGTDWVVID
VRGLLRNARWRTIAPKDISLNALLDLFTGDPVIDVRRNIVTFRYTLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFTPAPKKGAKKKAPVE
VMRKDRTWARAYKPRLSVEAQKLKNEALWALKRTSPEYKKLSRRKEELCRRSINY
VIEKTRRRTQCQIVIPVIEDLNVRFFHGRGKRLPGWDNFFTAKKENRWFIQGLHK
AFSDLRTHRSFYVFEVRPERTSITCPKCGHCEVGNRKGEAFQCLSCGKTCNADLD
VATHNLTQVALTGKTMPKREEPRDAQGTAPARKTKKASKSKAPPAEREDQTPAQE
PSQTSGGSKRTADGSEFEPKKKRKV
CasPhi2-HEPTA2 (D134R-L149R-D167K-T355R-T357K-L571K-D679K), dual
bpNLS (pEH1507)
(SEQ ID NO: 20)
MKRTADGSEFESPKKKRKVPKPAVESEFSKVLKKHFPGERFRSSYMKRGGKILAA
QGEEAVVAYLQGKSEEEPPNFQPPAKCHVVTKSRDFAEWPIMKASEAIQRYIYAL
STTERAACKPGKSSESHAAWFAATGVSNHGYSHVQGLNLIFRHTLGRYDGVLKKV
QRRNEKARARLESINASRAKEGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSPKKNKRMRRYWRSEKEKAQDALLVTVRIGTDWVVID
VRGLLRNARWRTIAPKDISLNALLDLFTGDPVIDVRRNIVTFRYKLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFTPAPKKGAKKKAPVE
VMRKDRTWARAYKPRLSVEAQKLKNEALWALKRTSPEYKKLSRRKEELCRRSINY
VIEKTRRRTQCQIVIPVIEDLNVRFFHGSGKRLPGWDNFFTAKKENRWFIQGLHK
AFSDLRTHRSFYVFEVRPERTSITCPKCGHCEVGNRKGEAFQCLSCGKTCNADLD
VATHNLTQVALTGKTMPKREEPRDAQGTAPARKTKKASKSKAPPAEREDQTPAQE
PSQTSGGSKRTADGSEFEPKKKRKV
CasPhi2-OCTA1 (A36R-L149R-D167K-T355R-T357K-L571K-S616R-D679K), dual
bpNLS (pEH1451)
(SEQ ID NO: 21)
MKRTADGSEFESPKKKRKVPKPAVESEFSKVLKKHFPGERFRSSYMKRGGKILRA
QGEEAVVAYLQGKSEEEPPNFQPPAKCHVVTKSRDFAEWPIMKASEAIQRYIYAL
STTERAACKPGKSSESHAAWFAATGVSNHGYSHVQGLNLIFDHTLGRYDGVLKKV
QRRNEKARARLESINASRAKEGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSPKKNKRMRRYWRSEKEKAQDALLVTVRIGTDWVVID
VRGLLRNARWRTIAPKDISLNALLDLFTGDPVIDVRRNIVTFRYKLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFTPAPKKGAKKKAPVE
VMRKDRTWARAYKPRLSVEAQKLKNEALWALKRTSPEYKKLSRRKEELCRRSINY
VIEKTRRRTQCQIVIPVIEDLNVRFFHGRGKRLPGWDNFFTAKKENRWFIQGLHK
AFSDLRTHRSFYVFEVRPERTSITCPKCGHCEVGNRKGEAFQCLSCGKTCNADLD
VATHNLTQVALTGKTMPKREEPRDAQGTAPARKTKKASKSKAPPAEREDQTPAQE
PSQTSGGSKRTADGSEFEPKKKRKV
CasPhi2-OCTA2 (A36R-L149R-D167K-T355R-L571K-S616R-D679K-Q684R), dual
bpNLS (pEH1460)
(SEQ ID NO: 22)
MKRTADGSEFESPKKKRKVPKPAVESEFSKVLKKHFPGERFRSSYMKRGGKILRA
QGEEAVVAYLQGKSEEEPPNFQPPAKCHVVTKSRDFAEWPIMKASEAIQRYIYAL
STTERAACKPGKSSESHAAWFAATGVSNHGYSHVQGLNLIFDHTLGRYDGVLKKV
QRRNEKARARLESINASRAKEGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSPKKNKRMRRYWRSEKEKAQDALLVTVRIGTDWVVID
VRGLLRNARWRTIAPKDISLNALLDLFTGDPVIDVRRNIVTFRYTLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFTPAPKKGAKKKAPVE
VMRKDRTWARAYKPRLSVEAQKLKNEALWALKRTSPEYKKLSRRKEELCRRSINY
VIEKTRRRTQCQIVIPVIEDLNVRFFHGRGKRLPGWDNFFTAKKENRWFIQGLHK
AFSDLRTHRSFYVFEVRPERTSITCPKCGHCEVGNRKGEAFRCLSCGKTCNADLD
VATHNLTQVALTGKTMPKREEPRDAQGTAPARKTKKASKSKAPPAEREDQTPAQE
PSQTSGGSKRTADGSEFEPKKKRKV
CasPhi2-NONA (A36R-L149R-D167K-P277R-T355R-T357K-L571K-S616R-D679K),
dual bpNLS (pEH1494)
(SEQ ID NO: 23)
MKRTADGSEFESPKKKRKVPKPAVESEFSKVLKKHFPGERFRSSYMKRGGKILRA
QGEEAVVAYLQGKSEEEPPNFQPPAKCHVVTKSRDFAEWPIMKASEAIQRYIYAL
STTERAACKPGKSSESHAAWFAATGVSNHGYSHVQGLNLIFDHTLGRYDGVLKKV
QRRNEKARARLESINASRAKEGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSRKKNKRMRRYWRSEKEKAQDALLVTVRIGTDWVVID
VRGLLRNARWRTIAPKDISLNALLDLFTGDPVIDVRRNIVTFRYKLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFTPAPKKGAKKKAPVE
VMRKDRTWARAYKPRLSVEAQKLKNEALWALKRTSPEYKKLSRRKEELCRRSINY
VIEKTRRRTQCQIVIPVIEDLNVRFFHGRGKRLPGWDNFFTAKKENRWFIQGLHK
AFSDLRTHRSFYVFEVRPERTSITCPKCGHCEVGNRKGEAFQCLSCGKTCNADLD
VATHNLTQVALTGKTMPKREEPRDAQGTAPARKTKKASKSKAPPAEREDQTPAQE
PSQTSGGSKRTADGSEFEPKKKRKV
CasPhi2-UNDECA (A36R-S106R-D134R-L149R-D167K-P277R-T355R-T357K-
L571K-S616R-D679K), dual bpNLS (pEH1834)
(SEQ ID NO: 24)
MKRTADGSEFESPKKKRKVPKPAVESEFSKVLKKHFPGERFRSSYMKRGGKILRA
QGEEAVVAYLQGKSEEEPPNFQPPAKCHVVTKSRDFAEWPIMKASEAIQRYIYAL
STTERAACKPGKSRESHAAWFAATGVSNHGYSHVQGLNLIFRHTLGRYDGVLKKV
QRRNEKARARLESINASRAKEGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSRKKNKRMRRYWRSEKEKAQDALLVTVRIGTDWVVID
VRGLLRNARWRTIAPKDISLNALLDLFTGDPVIDVRRNIVTFRYKLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFTPAPKKGAKKKAPVE
VMRKDRTWARAYKPRLSVEAQKLKNEALWALKRTSPEYKKLSRRKEELCRRSINY
VIEKTRRRTQCQIVIPVIEDLNVRFFHGRGKRLPGWDNFFTAKKENRWFIQGLHK
AFSDLRTHRSFYVFEVRPERTSITCPKCGHCEVGNRKGEAFQCLSCGKTCNADLD
VATHNLTQVALTGKTMPKREEPRDAQGTAPARKTKKASKSKAPPAEREDQTPAQE
PSQTSGGSKRTADGSEFEPKKKRKV
CasPhi2-DODECA1 (S106R-D134R-L149R-D167K-P277R-T355R-T357K-T518R-
L571K-D679K-Q684R-T691R), dual bpNLS (pEH1726)
(SEQ ID NO: 25)
MKRTADGSEFESPKKKRKVPKPAVESEFSKVLKKHFPGERFRSSYMKRGGKILAA
QGEEAVVAYLQGKSEEEPPNFQPPAKCHVVTKSRDFAEWPIMKASEAIQRYIYAL
STTERAACKPGKSRESHAAWFAATGVSNHGYSHVQGLNLIFRHTLGRYDGVLKKV
QRRNEKARARLESINASRAKEGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSRKKNKRMRRYWRSEKEKAQDALLVTVRIGTDWVVID
VRGLLRNARWRTIAPKDISLNALLDLFTGDPVIDVRRNIVTFRYKLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFRPAPKKGAKKKAPVE
VMRKDRTWARAYKPRLSVEAQKLKNEALWALKRTSPEYKKLSRRKEELCRRSINY
VIEKTRRRTQCQIVIPVIEDLNVRFFHGSGKRLPGWDNFFTAKKENRWFIQGLHK
AFSDLRTHRSFYVFEVRPERTSITCPKCGHCEVGNRKGEAFRCLSCGKRCNADLD
VATHNLTQVALTGKTMPKREEPRDAQGTAPARKTKKASKSKAPPAEREDQTPAQE
PSQTSGGSKRTADGSEFEPKKKRKV
CasPhi2-DODECA2 (A36R-S106R-D134R-L149R-D167K-P277R-T355R-T357K-
T518R-L571K-S616R-D679K), dual bpNLS (pEH1844)
(SEQ ID NO: 26)
MKRTADGSEFESPKKKRKVPKPAVESEFSKVLKKHFPGERFRSSYMKRGGKILRA
QGEEAVVAYLQGKSEEEPPNFQPPAKCHVVTKSRDFAEWPIMKASEAIQRYIYAL
STTERAACKPGKSRESHAAWFAATGVSNHGYSHVQGLNLIFRHTLGRYDGVLKKV
QRRNEKARARLESINASRAKEGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSRKKNKRMRRYWRSEKEKAQDALLVTVRIGTDWVVID
VRGLLRNARWRTIAPKDISLNALLDLFTGDPVIDVRRNIVTFRYKLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFRPAPKKGAKKKAPVE
VMRKDRTWARAYKPRLSVEAQKLKNEALWALKRTSPEYKKLSRRKEELCRRSINY
VIEKTRRRTQCQIVIPVIEDLNVRFFHGRGKRLPGWDNFFTAKKENRWFIQGLHK
AFSDLRTHRSFYVFEVRPERTSITCPKCGHCEVGNRKGEAFQCLSCGKTCNADLD
VATHNLTQVALTGKTMPKREEPRDAQGTAPARKTKKASKSKAPPAEREDQTPAQE
PSQTSGGSKRTADGSEFEPKKKRKV
CasPhi2-DODECA3 (A36R-S106R-D134R-L149R-D167K-P277R-T355R-T357K-
L571K-S616R-D679K-Q684R), dual bpNLS (pEH1848)
(SEQ ID NO: 27)
MKRTADGSEFESPKKKRKVPKPAVESEFSKVLKKHFPGERFRSSYMKRGGKILRA
QGEEAVVAYLQGKSEEEPPNFQPPAKCHVVTKSRDFAEWPIMKASEAIQRYIYAL
STTERAACKPGKSRESHAAWFAATGVSNHGYSHVQGLNLIFRHTLGRYDGVLKKV
QRRNEKARARLESINASRAKEGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSRKKNKRMRRYWRSEKEKAQDALLVTVRIGTDWVVID
VRGLLRNARWRTIAPKDISLNALLDLFTGDPVIDVRRNIVTFRYKLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFTPAPKKGAKKKAPVE
VMRKDRTWARAYKPRLSVEAQKLKNEALWALKRTSPEYKKLSRRKEELCRRSINY
VIEKTRRRTQCQIVIPVIEDLNVRFFHGRGKRLPGWDNFFTAKKENRWFIQGLHK
AFSDLRTHRSFYVFEVRPERTSITCPKCGHCEVGNRKGEAFRCLSCGKTCNADLD
VATHNLTQVALTGKTMPKREEPRDAQGTAPARKTKKASKSKAPPAEREDQTPAQE
PSQTSGGSKRTADGSEFEPKKKRKV
CasPhi2-HEXADECA (16AA) (A36R-S106R-D134R-L149R-E159A-S160A-S164A-
D167K-E168A-P277R-T355R-T357K-L571K-S616R-D679K-Q684R), dual bpNLS
(pEH1880)
(SEQ ID NO: 28)
MKRTADGSEFESPKKKRKVPKPAVESEFSKVLKKHFPGERFRSSYMKRGGKILRA
QGEEAVVAYLQGKSEEEPPNFQPPAKCHVVTKSRDFAEWPIMKASEAIQRYIYAL
STTERAACKPGKSRESHAAWFAATGVSNHGYSHVQGLNLIFRHTLGRYDGVLKKV
QRRNEKARARLAAINAARAKAGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSRKKNKRMRRYWRSEKEKAQDALLVTVRIGTDWVVID
VRGLLRNARWRTIAPKDISLNALLDLFTGDPVIDVRRNIVTFRYKLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFTPAPKKGAKKKAPVE
VMRKDRTWARAYKPRLSVEAQKLKNEALWALKRTSPEYKKLSRRKEELCRRSINY
VIEKTRRRTQCQIVIPVIEDLNVRFFHGRGKRLPGWDNFFTAKKENRWFIQGLHK
AFSDLRTHRSFYVFEVRPERTSITCPKCGHCEVGNRKGEAFRCLSCGKTCNADLD
VATHNLTQVALTGKTMPKREEPRDAQGTAPARKTKKASKSKAPPAEREDQTPAQE
PSQTSGGSKRTADGSEFEPKKKRKV
CasPhi2-HEPTADECA (17AA) (A36R-S106R-D134R-L149R-E159A-S160A-S164A-
D167K-E168A-P277R-T355R-T357K-T518R-L571K-S616R-D679K-Q684R), dual
bpNLS (pEH1869)
(SEQ ID NO: 29)
MKRTADGSEFESPKKKRKVPKPAVESEFSKVLKKHFPGERFRSSYMKRGGKILRA
QGEEAVVAYLQGKSEEEPPNFQPPAKCHVVTKSRDFAEWPIMKASEAIQRYIYAL
STTERAACKPGKSRESHAAWFAATGVSNHGYSHVQGLNLIFRHTLGRYDGVLKKV
QRRNEKARARLAAINAARAKAGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSRKKNKRMRRYWRSEKEKAQDALLVTVRIGTDWVVID
VRGLLRNARWRTIAPKDISLNALLDLFTGDPVIDVRRNIVTFRYKLDACGTYARK
WTLKGKQTKATLDKLTATQTVALVAIDLGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFRPAPKKGAKKKAPVE
VMRKDRTWARAYKPRLSVEAQKLKNEALWALKRTSPEYKKLSRRKEELCRRSINY
VIEKTRRRTQCQIVIPVIEDLNVRFFHGRGKRLPGWDNFFTAKKENRWFIQGLHK
AFSDLRTHRSFYVFEVRPERTSITCPKCGHCEVGNRKGEAFRCLSCGKTCNADLD
VATHNLTQVALTGKTMPKREEPRDAQGTAPARKTKKASKSKAPPAEREDQTPAQE
PSQTSGGSKRTADGSEFEPKKKRKV
ABE-dCasPhi2-17AA (TadA8e-32AA linker-dead(D394A)CasPhi2-17AA; CasPhi2
with the following mutations: A36R-S106R-D134R-L149R-E159A-S160A-S164A-
D167K-E168A-P277R-T355R-T357K-D394A-T518R-L571K-S616R-D679K-Q684R),
dual bpNLS (pBM3865)
(SEQ ID NO: 30)
MKRTADGSEFESPKKKRKVSEVEFSHEYWMRHALTLAKRARDEREVPVGAVLVLN
NRVIGEGWNRAIGLHDPTAHAEIMALRQGGLVMQNYRLIDATLYVTFEPCVMCAG
AMIHSRIGRVVFGVRNSKRGAAGSLMNVLNYPGMNHRVEITEGILADECAALLCD
FYRMPRQVFNAQKKAQSSINSGGSSGGSSGSETPGTSESATPESSGGSSGGSPKP
AVESEFSKVLKKHFPGERFRSSYMKRGGKILRAQGEEAVVAYLQGKSEEEPPNFQ
PPAKCHVVTKSRDFAEWPIMKASEAIQRYIYALSTTERAACKPGKSRESHAAWFA
ATGVSNHGYSHVQGLNLIFRHTLGRYDGVLKKVQRRNEKARARLAAINAARAKAG
LPEIKAEEEEVATNETGHLLQPPGINPSFYVYQTISPQAYRPRDEIVLPPEYAGY
VRDPNAPIPLGVVRNRCDIQKGCPGYIPEWQREAGTAISPKTGKAVTVPGLSRKK
NKRMRRYWRSEKEKAQDALLVTVRIGTDWVVIDVRGLLRNARWRTIAPKDISLNA
LLDLFTGDPVIDVRRNIVTFRYKLDACGTYARKWTLKGKQTKATLDKLTATQTVA
LVAIALGQTNPISAGISRVTQENGALQCEPLDRFTLPDDLLKDISAYRIAWDRNE
EELRARSVEALPEAQQAEVRALDGVSKETARTQLCADFGLDPKRLPWDKMSSNTT
FISEALLSNSVSRDQVFFRPAPKKGAKKKAPVEVMRKDRTWARAYKPRLSVEAQK
LKNEALWALKRTSPEYKKLSRRKEELCRRSINYVIEKTRRRTQCQIVIPVIEDLN
VRFFHGRGKRLPGWDNFFTAKKENRWFIQGLHKAFSDLRTHRSFYVFEVRPERTS
ITCPKCGHCEVGNRKGEAFRCLSCGKTCNADLDVATHNLTQVALTGKTMPKREEP
RDAQGTAPARKTKKASKSKAPPAEREDQTPAQEPSQTSGGSKRTADGSEFEPKKK
RKV
dCasPhi2-17AA-VPR (dead(D394A)CasPhi2-17AA-32AA linker-VPR; CasPhi2 with
the following mutations: A36R-S106R-D134R-L149R-E159A-S160A-S164A-D167K-
E168A-P277R-T355R-T357K-D394A-T518R-L571K-S616R-D679K-Q684R), dual
bpNLS (pBM3891)
(SEQ ID NO: 31)
MKRTADGSEFESPKKKRKVPKPAVESEFSKVLKKHFPGERFRSSYMKRGGKILRA
QGEEAVVAYLQGKSEEEPPNFQPPAKCHVVTKSRDFAEWPIMKASEAIQRYIYAL
STTERAACKPGKSRESHAAWFAATGVSNHGYSHVQGLNLIFRHTLGRYDGVLKKV
QRRNEKARARLAAINAARAKAGLPEIKAEEEEVATNETGHLLQPPGINPSFYVYQ
TISPQAYRPRDEIVLPPEYAGYVRDPNAPIPLGVVRNRCDIQKGCPGYIPEWQRE
AGTAISPKTGKAVTVPGLSRKKNKRMRRYWRSEKEKAQDALLVTVRIGTDWVVID
VRGLLRNARWRTIAPKDISLNALLDLFTGDPVIDVRRNIVTFRYKLDACGTYARK
WTLKGKQTKATLDKLTATQTVALVAIALGQTNPISAGISRVTQENGALQCEPLDR
FTLPDDLLKDISAYRIAWDRNEEELRARSVEALPEAQQAEVRALDGVSKETARTQ
LCADFGLDPKRLPWDKMSSNTTFISEALLSNSVSRDQVFFRPAPKKGAKKKAPVE
VMRKDRTWARAYKPRLSVEAQKLKNEALWALKRTSPEYKKLSRRKEELCRRSINY
VIEKTRRRTQCQIVIPVIEDLNVRFFHGRGKRLPGWDNFFTAKKENRWFIQGLHK
AFSDLRTHRSFYVFEVRPERTSITCPKCGHCEVGNRKGEAFRCLSCGKTCNADLD
VATHNLTQVALTGKTMPKREEPRDAQGTAPARKTKKASKSKAPPAEREDQTPAQE
PSQTSGSPKKKRKVKRPAATKKAGQAKKKKGSYPYDVPDYAYPYDVPDYAYPYDV
PDYAGSEASGSGRADALDDFDLDMLGSDALDDFDLDMLGSDALDDFDLDMLGSDA
LDDFDLDMLINSRSSGSPKKKRKVGSQYLPDTDDRHRIEEKRKRTYETFKSIMKK
SPFSGPTDPRPPPRRIAVPSRSSASVPKPAPQPYPFTSSLSTINYDEFPTMVFPS
GQISQASALAPAPPQVLPQAPAPAPAPAMVSALAQAPAPVPVLAPGPPQAVAPPA
PKPTQAGEGTLSEALLQLQFDDEDLGALLGNSTDPAVFTDLASVDNSEFQQLLNQ
GIPVAPHTTEPMLMEYPEAITRLVTGAQRPPDPAPAPLGAPGLPNGLLSGDEDFS
SIADMDFSALLGSGSGSRDSREGMFLPKPEAGSAISDVFEGREVCQPKRIRPFHP
PGSPWANRPLPASLAPTPTGPVHEPVGSLTPAPVPQPLDPAPAVTPEASHLLEDP
DEETSQAVKALREMADTVIPQKEEAAICGQMDLSHPPPRGHLDELTTTLESMTED
LNLDSPLTPELNEILDTFLNDECLLHAMHISTGLSIFDTSLF

### REFERENCES

1. Jinek, M. et al. A Programmable Dual-RNA-Guided DNA Endonuclease in Adaptive Bacterial Immunity. Science 337, 816-821 (2012).
2. Zetsche, B. et al. Cpf1 is a single RNA-guided endonuclease of a class 2 CRISPR-Cas system. Cell 163, 759-771 (2015).
3. Barrangou, R. & Marraffini, L. A. CRISPR-Cas systems: Prokaryotes upgrade to adaptive immunity. Mol. Cell 54, 234-244 (2014).
4. Anzalone, A. V., Koblan, L. W. & Liu, D. R. Genome editing with CRISPR-Cas nucleases, base editors, transposases and prime editors. Nat. Biotechnol. 38, 824-844 (2020).
5. Pickar-Oliver, A. & Gersbach, C. A. The next generation of CRISPR-Cas technologies and applications. Nat. Rev. Mol. Cell Biol. 20, 490-507 (2019).
6. McGaw, C. et al. Engineered Cas1212 is a versatile high-efficiency platform for therapeutic genome editing. Nat. Commun. 13, 2833 (2022).
7. Zhang, H. et al. An engineered xCas12i with high activity, high specificity and broad PAM range. http://biorxiv.org/lookup/doi/10.1101/2022.06.15.496255 (2022) doi: 10.1101/2022.06.15.496255.
8. Harrington, L. B. et al. Programmed DNA destruction by miniature CRISPR-Cas14 enzymes. Science 362, 839-842 (2018).
9. Wu, Z. et al. Programmed genome editing by a miniature CRISPR-Cas12f nuclease. Nat. Chem. Biol. 17, 1132-1138 (2021).
10. Xu, X. et al. Engineered miniature CRISPR-Cas system for mammalian genome regulation and editing. Mol. Cell 81, 4333-4345.e4 (2021).
11. Karvelis, T. et al. PAM recognition by miniature CRISPR-Cas12f nucleases triggers programmable double-stranded DNA target cleavage. Nucleic Acids Res. 48, 5016-5023 (2020).
12. Takeda, S. N. et al. Structure of the miniature type V-F CRISPR-Cas effector enzyme. Mol. Cell 81, 558-570.e3 (2021).
13. Karvelis, T. et al. Transposon-associated TnpB is a programmable RNA-guided DNA endonuclease. Nature 599, 692-696 (2021).
14. Kim, D. Y. et al. Hypercompact adenine base editors based on transposase B guided by engineered RNA. Nat. Chem. Biol. 18, 1005-1013 (2022).
15. Pausch, P. et al. CRISPR-CasΦ from huge phages is a hypercompact genome editor. Science 369, 333-337 (2020).
16. Pausch, P. et al. DNA interference states of the hypercompact CRISPR-CasΦ effector. Nat. Struct. Mol. Biol. 28, 652-661 (2021).
17. Xin, C. et al. Comprehensive assessment of miniature CRISPR-Cas12f nucleases for gene disruption. Nat. Commun. 13, 5623 (2022).
18. Kaminski, M. M., Abudayyeh, O. O., Gootenberg, J. S., Zhang, F. & Collins, J. J. CRISPR-based diagnostics. Nat. Biomed. Eng. 5, 643-656 (2021).
19. Kellner, M. J., Koob, J. G., Gootenberg, J. S., Abudayyeh, O. O. & Zhang, F. SHERLOCK: nucleic acid detection with CRISPR nucleases. Nat. Protoc. 14, 2986-3012 (2019).
20 Chen, J. S. et al. CRISPR-Cas12a target binding unleashes indiscriminate single-stranded DNase activity. Science 360, 436-439 (2018).
21 Escobar, M. et al. Quantification of Genome Editing and Transcriptional Control Capabilities Reveals Hierarchies among Diverse CRISPR/Cas Systems in Human Cells. ACS Synth. Biol. (2022) doi: 10.1021/acssynbio.2c00156.
22. Kleinstiver, B. P. et al. Engineered CRISPR-Cas12a variants with increased activities and improved targeting ranges for gene, epigenetic and base editing. Nat. Biotechnol. 37, 276-282 (2019).
23. Strecker, J. et al. Engineering of CRISPR-Cas12b for human genome editing. Nat. Commun. 10, 212 (2019).
24. Richter, M. F. et al. Phage-assisted evolution of an adenine base editor with improved Cas domain compatibility and activity. Nat. Biotechnol. 38, 883-891 (2020).
25. Wu, Y. et al. Highly efficient therapeutic gene editing of human hematopoietic stem cells. Nat. Med. 25, 776-783 (2019).
26. Frangoul, H. et al. CRISPR-Cas9 Gene Editing for Sickle Cell Disease and β-Thalassemia. N. Engl. J. Med. 384, 252-260 (2021).
27 Komor, A. C. et al. Improved base excision repair inhibition and bacteriophage Mu Gam protein yields C: G-to-T: A base editors with higher efficiency and product purity. Sci. Adv. 3, eaao4774 (2017).
   28. Gaudelli, N. M. et al. Programmable base editing of A·T to G·C in genomic DNA without DNA cleavage. Nature 551, 464-471 (2017).
   29. Anzalone, A. V. et al. Search-and-replace genome editing without double-strand breaks or donor DNA. Nature 576, 149-157 (2019).
   30. Holtzman, L. & Gersbach, C. A. Editing the Epigenome: Reshaping the Genomic Landscape. Annu. Rev. Genomics Hum. Genet. 19, 43-71 (2018).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated CasPhi2 protein, comprising an amino acid sequence having at least 70%, 75%, 80%, 85%, 90%, or 95% sequence identity to the amino acid sequence of SEQ ID NO: 1, and comprising a mutation at one or more of the following positions: S11, S25, A36, S106, E107, S124, D134, G138, L149, A156, E159, S160, S164, D167, E168, T203, P233, D337, A261, P277, T355, T357, L370, D427, D428, A435, N497, L506, S507, N508, S509, S511, D513, Q514, T518, P519, A520, P521, G524, A525, K526, K527, P530, V531, E532, V533, R538, T539, E569, L571, S574, E578, S616, T628, T649, D679, Q684, and/or T691.

2. An isolated CasPhi2 protein, comprising an amino acid sequence having at least 70%, 75%, 80%, 85%, 90%, or 95% sequence identity to the amino acid sequence of SEQ ID NO:1, and comprising a mutation at one or more of the following positions: T355 and/or D679.

3. The isolated CasPhi2 protein of claim 2, further comprising a mutation at one or more of the following positions: S11, S25, A36, S106, E107, S124, D134, G138, L149, A156, E159, S160, S164, D167, E168, T203, P233, A261, P277, D337, T357, L370, D427, D428, A435, N497, L506, S507, N508, S509, S511, D513, Q514, T518, P519, A520, P521, G524, A525, K526, K527, P530, V531, E532, V533, R538, T539, A543 E569, L571, S574, E578, S616, T628, T649, E674, Q684, and/or T691.

4. The isolated CasPhi2 protein of any one of claims 1-3, wherein the CasPhi2 protein comprises a mutation at T355 and the mutation is T355R or T355K.

5. The isolated CasPhi2 protein of any one of claims 1-4, wherein the CasPhi2 protein comprises a mutation at D679 and the mutation is D679R, D679K, D679H, or D679T.

6. The isolated CasPhi2 protein of any one of claims 1-5, comprising one of the combinations of mutations listed in Table 1.

7. The isolated CasPhi2 protein of claim 1, comprising the following mutations: A36R, S106R, D134R, L149R, E159A, S160A, S164A, D167K, E168A, P277R, T357K, T518R, L571K, S616R, Q684R, T355R, and D679K.

8. The isolated CasPhi2 protein of claim 1, comprising the following mutations: A36R, S106R, D134R, P277R, T355R, T357K, T518R, L571K, S616R, D679K, and Q684R.

9. The isolated CasPhi2 protein of claim 8, further comprising a mutation at one or more of the following positions: S11, S25, G138, T203, A261, D337, N497, L506, S507, N508, S509, D513, Q514, A520, G524, A525, K527, P530, V531, R538, T539, R542, A543, E569, E578, T628, T649, E674, and/or T691.

10. The isolated CasPhi2 protein of claim 8, further comprising the following mutations: F23S and S26R.

11. The isolated CasPhi2 protein of claim 8, further comprising the following mutations: T340G, D341R, and D342G.

12. The isolated CasPhi2 protein of claim 1, comprising the following mutations: A36R, S106R, D134R, L149R, P277R, T355R, T357K, T518R, L571K, S616R, D679K, and Q684R.

13. The isolated CasPhi2 protein of claim 1, comprising the following mutations: A36K, S106K, D134K, P277K, D337K, T355R, T357K, V531R, T539A, A543K, L571K, S616K, D679K, and T691K.

14. The isolated CasPhi2 protein of claim 13, further comprising the following mutation: Q684R.

15. The isolated CasPhi2 protein of claims 1-14, further comprising a mutation that catalytically inactivates nuclease activity, wherein the mutation is D394A of SEQ ID NO: 1.

16. The isolated CasPhi2 protein of claims 1-14, further comprising a mutation that catalytically impairs nuclease activity, wherein the mutation is E606Q of SEQ ID NO:1.

17. A fusion protein comprising isolated CasPhi2 protein of any one of claims 1-16, fused to at least one heterologous functional domain, with an optional intervening linker, wherein the linker does not interfere with activity of the fusion protein.

18. The fusion protein of claim 17, wherein the heterologous functional domain is a transcriptional activation domain.

19. The fusion protein of claim 18, wherein the transcriptional activation domain is VP16, VP64, Rta, NF-κB p65, p300, or a VPR fusion.

20. The fusion protein of claim 17, wherein the heterologous functional domain is a transcriptional silencer or transcriptional repression domain.

21. The fusion protein of claim 20, wherein the transcriptional repression domain is a Krueppel-associated box (KRAB) domain, ERF repressor domain (ERD), or mSin3A interaction domain (SID).

22. The fusion protein of claim 20, wherein the transcriptional silencer is Heterochromatin Protein 1 (HP1).

23. The fusion protein of claim 17, wherein the heterologous functional domain is an enzyme that modifies the methylation state of DNA.

24. The fusion protein of claim 23, wherein the enzyme that modifies the methylation state of DNA is a DNA methyltransferase (DNMT) or a TET protein.

25. The fusion protein of claim 24, wherein the TET protein is TET1.

26. The fusion protein of claim 17, wherein the heterologous functional domain is an enzyme that modifies a histone subunit.

27. The fusion protein of claim 26, wherein the enzyme that modifies a histone subunit is a histone acetyltransferase (HAT), histone deacetylase (HDAC), histone methyltransferase (HMT), or histone demethylase.

28. The fusion protein of claim 17, wherein the heterologous functional domain is a biological tether.

29. The fusion protein of claim 28, wherein the biological tether is MS2, Csy4 or lambda N protein.

30. The fusion protein of claim 17, wherein the heterologous functional domain is FokI.

31. The fusion protein of claim 17, wherein the heterologous functional domain is a deaminase.

32. The fusion protein of claim 31, wherein the heterologous functional domain is a cytidine deaminase.

33. The fusion protein of claim 32, wherein the cytidine deaminase is selected from the group consisting of APOBEC1, APOBEC2, APOBEC3A, APOBEC3B, APOBEC3C, APOBEC3D/E, APOBEC3F, APOBEC3G, APOBEC3H, APOBEC4, activation-induced cytidine deaminase (AID), cytosine deaminase 1 (CDA1), pmCDA1, CDA2, and cytosine deaminase acting on tRNA (CDAT).

34. The fusion protein of claim 31, wherein the heterologous functional domain is an adenosine deaminase.

35. The fusion protein of claim 34, wherein the adenosine deaminase is selected from the group consisting of adenosine deaminase 1 (ADA1), ADA2; adenosine deaminase acting on RNA 1 (ADAR1), ADAR2, ADAR3; adenosine deaminase acting on tRNA 1 (ADAT1), ADAT2, ADAT3; and naturally occurring or engineered tRNA-specific adenosine deaminase (TadA).

36. The fusion protein of any one of claims 17 or 31 to 35, comprising at least two heterologous functional domains, wherein the additional heterologous functional domain comprises an enzyme, domain, or peptide that inhibits or enhances endogenous DNA repair or base excision repair (BER) pathways.

37. The fusion protein of claim 36, wherein the additional heterologous functional domain is a uracil DNA glycosylase inhibitor (UGI) that inhibits uracil DNA glycosylase (UDG, also known as uracil N-glycosylase, or UNG); or Gam from the bacteriophage Mu.

38. An isolated nucleic acid encoding the isolated CasPhi2 protein of any one of claims 1-8 or the fusion protein of claims 17-37.

39. A vector comprising the isolated nucleic acid of claim 38.

40. An isolated host cell comprising the nucleic acid of claim 39.

41. The isolated host cell of claim 40, wherein the host cell is a mammalian host cell.

42. A composition comprising:
An isolated nucleic acid encoding the isolated CasPhi2 protein of any one of claims 1-16 or the fusion protein of claims 17-37; and
a nucleic acid comprising or encoding one or more crRNAs or pre-crRNAs, optionally an array of two or more pre-crRNAs.

43. The composition of claim 42, wherein the one or more crRNAs or pre-crRNAs direct the isolated CasPhi2 protein to one or more target genomic sequences.

44. The composition of any one of claims 42-43, wherein one or more crRNAs or pre-crRNAs includes a complementarity region that is complementary to 14-24 nucleotides of a respective target genomic sequence or sequences.

45. The composition of any one of claims 42-44, wherein the one or more crRNAs or pre-crRNAs comprises the following sequence:
5′-CAACGAUUGCCCCUCACGAGGGGAC-N₁₂₋₂₄-U₀₋₈, SEQ ID NO: 104,
5′-GUCGGAACGCUCAACGAUUGCCCCUCACGAGGGGAC-N₁₂₋₂₄-U₀₋₈, SEQ ID NO: 105,
5′-GCAACGAUUGCCCCUCACGAGGGGAC-N₁₂₋₂₄-U₀₋₈, SEQ ID NO: 106,
5′-GGUCGGAACGCUCAACGAUUGCCCCUCACGAGGGGAC-N₁₂₋₂₄-U₀₋₈, SEQ ID NO: 107,
5′-GGCAACGAUUGCCCCUCACGAGGGGAC-N₁₂₋₂₄-U₀₋₈, SEQ ID NO: 108, or
5′-GGGUCGGAACGCUCAACGAUUGCCCCUCACGAGGGGAC-N₁₂₋₂₄-U₀₋₈, SEQ-ID No. 109, and
wherein N is any nucleotide, and wherein the one or more crRNAs or pre-crRNAs is designed to be complementary to the respective target genomic sequence or sequences.

46. A method of altering a genome of a cell, the method comprising expressing in the cell, or contacting the cell with, the isolated CasPhi2 protein of any one of claims 1-16 or the fusion protein of any one of claims 17-36, and one or more crRNAs or pre-crRNAs or a nucleic acid comprising or encoding one or more crRNAs or pre-crRNAs, optionally an array of two or more pre-crRNAs, wherein the one or more crRNAs or pre-crRNAs direct the isolated CasPhi2 protein of any one of claims 1-16 or the fusion protein of any one of claims 17-36 to one or more target genomic sequences.

47. The method of claim 46, wherein the cell is a stem cell.

48. The method of claim 47, wherein the stem cell is an embryonic stem cell, a mesenchymal stem cell, or an induced pluripotent stem cell; is in a living animal; or is in or is an embryo.

49. A method of altering a double stranded DNA (dsDNA) molecule, the method comprising contacting the dsDNA with the isolated CasPhi2 protein of any one of claims 1-16 or the fusion protein of any one of claims 17-36, and one or more crRNAs or pre-crRNAs or a nucleic acid comprising or encoding one or more crRNAs or pre-crRNAs, optionally an array of two or more pre-crRNAs, wherein the one or more crRNAs or pre-crRNAs direct the isolated CasPhi2 protein of any one of claims 1-16 or the fusion protein of any one of claims 17-36 to one or more target genomic sequences.

50. The method of claim 49, wherein the dsDNA molecule is in vitro.

51. The method of any one of claims 46-50, wherein the one or more crRNAs or pre-crRNAs includes a complementarity region that is complementary to 14-24 nucleotides of the one or more target genomic sequences.

52. The method of any one of claims 46-51, wherein the one or more crRNAs or pre-crRNAs comprises the following sequence:
5′-CAACGAUUGCCCCUCACGAGGGGAC-N₁₂₋₂₄-U₀₋₈, SEQ ID NO: 104,
5′-GUCGGAACGCUCAACGAUUGCCCCUCACGAGGGGAC-N₁₂₋₂₄-U₀₋₈, SEQ ID NO: 105,
5′-GCAACGAUUGCCCCUCACGAGGGGAC-N₁₂₋₂₄-U₀₋₈, SEQ ID NO: 106,
5′-GGUCGGAACGCUCAACGAUUGCCCCUCACGAGGGGAC-N₁₂₋₂₄-U₀₋₈, SEQ ID NO: 107,
5′-GGCAACGAUUGCCCCUCACGAGGGGAC-N₁₂₋₂₄-U₀₋₈, SEQ ID NO: 108, or
5′-GGGUCGGAACGCUCAACGAUUGCCCCUCACGAGGGGAC-N₁₂₋₂₄-U₀₋₈, SEQ-ID No. 109, and
wherein N is any nucleotide, and wherein the one or more crRNAs or pre-crRNAs is designed to be complementary to the respective target genomic sequence or sequences.

53. The method of any one of claims 46-52, further comprising co-expressing and/or contacting an additional single- or double-stranded DNA donor (ssODN or dsODN) in the cell to enable homologous recombination or homology-directed repair with that ssODN or dsODN donor to introduce alterations, deletions, or insertions in the proximity of the site of the double-stranded break induced by the isolated CasPhi2 protein of any one of claims 1-8 or the fusion protein of any one of claims 9-29.

54. A kit comprising:
(a) the isolated CasPhi2 protein of any one of claims 1-16 or the fusion protein of any one of claims 17-36, or nucleic acids encoding the isolated CasPhi2 protein of any one of claims 1-16 or the fusion protein of any one of claims 17-36;
(b) one or more crRNAs or pre-crRNAs comprising one or more of the following sequences:
5′-CAACGAUUGCCCCUCACGAGGGGAC-N₁₂₋₂₄-U₀₋₈, SEQ ID NO: 104,
5′-GUCGGAACGCUCAACGAUUGCCCCUCACGAGGGGAC-N₁₂₋₂₄-U₀₋₈, SEQ ID NO: 105,
5′-GCAACGAUUGCCCCUCACGAGGGGAC-N₁₂₋₂₄-U₀₋₈, SEQ ID NO: 106,
5′-GGUCGGAACGCUCAACGAUUGCCCCUCACGAGGGGAC-N₁₂₋₂₄-U₀₋₈, SEQ ID NO: 107,
5′-GGCAACGAUUGCCCCUCACGAGGGGAC-N₁₂₋₂₄-U₀₋₈, SEQ ID NO: 108, or
5′-GGGUCGGAACGCUCAACGAUUGCCCCUCACGAGGGGAC-N₁₂₋₂₄-U₀₋₈, SEQ-ID No. 109, and wherein N is any nucleotide, and wherein the one or more crRNAs or pre-crRNAs is designed to be complementary to the respective target genomic sequence or sequences, or nucleic acids encoding the one or more crRNAs or pre-crRNAs; and
(c) a single-stranded DNA with a signal detectable upon cleavage.

55. A method of detecting a target DNA sequence in vitro, the method comprising:
incubating a DNA sample with:
(a) the isolated CasPhi2 protein of any one of claims 1-16 or the fusion protein of any one of claims 17-36;
(b) one or more crRNAs or pre-crRNAs comprising one or more of the following sequences:
5′-CAACGAUUGCCCCUCACGAGGGGAC-N₁₂₋₂₄-U₀₋₈, SEQ ID NO: 104,
5′-GUCGGAACGCUCAACGAUUGCCCCUCACGAGGGGAC-N₁₂₋₂₄-U₀₋₈, SEQ ID NO: 105,
5′-GCAACGAUUGCCCCUCACGAGGGGAC-N₁₂₋₂₄-U₀₋₈, SEQ ID NO: 106,
5′-GGUCGGAACGCUCAACGAUUGCCCCUCACGAGGGGAC-N₁₂₋₂₄-U₀₋₈, SEQ ID NO: 107,
5′-GGCAACGAUUGCCCCUCACGAGGGGAC-N₁₂₋₂₄-U₀₋₈, SEQ ID NO: 108, or
5′-GGGUCGGAACGCUCAACGAUUGCCCCUCACGAGGGGAC-N₁₂₋₂₄-U₀₋₈, SEQ-ID No. 109, and wherein N is any nucleotide, and wherein the one or more crRNAs or pre-crRNAs is designed to be complementary to the respective target genomic sequence or sequences; and
(c) a single-stranded DNA with a detectable signal upon cleavage, and determining the presence or absence of the detectable signal.

56. The method of claim 55, wherein two or more crRNAs designed to recognize two or more target DNA sequences are provided as pre-crRNAs encoded in a single array that are then processed into individual crRNAs by the isolated CasPhi2 protein of any one of claims 1-16 or the fusion protein of any one of claims 17-36.
